(19) United States
(12) Patent Application Publication
(10) Pub. No.: US 2022/0331132 A1
(43) Pub. Date: Oct. 20, 2022

(54) DEVICES AND METHODS FOR PROSTHETIC VALVE DIAMETER ESTIMATION

(71) Applicant: Edwards Lifesciences Corporation, Irvine, CA (US)

(72) Inventors: Oren Cohen, Kadima (IL); Elazar Levi Schwarcz, Netanya (IL); Ofir Witzman, Kfar Saba (IL); Eitan Atias, Tel Aviv (IL); Noam Miller, Givatayim (IL); Tomer Saar, Pardes Hanna-Karkur (IL); Noa Axelrod Manela, Netanya (IL); Eran Goldberg, Nesher (IL); Tal Reich, Moledet (IL)

(73) Assignee: Edwards Lifesciences Corporation, Irvine, CA (US)

(21) Appl. No.: 17/733,142

(22) Filed: Apr. 29, 2022

Related U.S. Application Data
(63) Continuation of application No. PCT/US2020/057502, filed on Oct. 27, 2020.
(60) Provisional application No. 62/928,320, filed on Oct. 30, 2019.

(51) Int. Cl. A61F 2/95; A61F 2/24

(52) U.S. Cl. CPC A61F 2/95; A61F 2/2418; A61F 2002/9511; A61F 2250/001; A61F 2250/0098

(57) ABSTRACT

The present invention relates to devices, assemblies and methods for monitoring radial expansion of a prosthetic valve during prosthetic valve implantation procedures.

### CROSS REFERENCE TO RELATED APPLICATIONS

This application is a continuation of International Application No. PCT/US2020/057502, filed Oct. 27, 2020, which claims benefit of U.S. Provisional Application No. 62/928,320, filed on Oct. 30, 2019, the contents of each of which are herein incorporated by reference in their entirety.

### FIELD OF THE INVENTION

The present invention relates to devices and methods for measuring prosthetic valve expansion diameter

### BACKGROUND OF THE INVENTION

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

Mechanically expandable valves are a category of prosthetic valves that rely on a mechanical actuation mechanism for expansion. The actuation mechanism usually includes a plurality of actuation/locking assemblies, releasably connected to respective actuation members of the valve delivery system, controlled via the handle for actuating the assemblies to expand the valve to a desired diameter. The assemblies may optionally lock the valve's position to prevent undesired recompression thereof, and disconnection of the delivery system's actuation member from the valve actuation/locking assemblies, to enable retrieval thereof once the valve is properly positioned at the desired site of implantation. Various types of re-compression assemblies may be utilized to re-compress an expanded prosthetic valve in order to allow repositioning or re-crossing procedures to be performed, and to allow readjustment of the prosthetic valve expansion diameter.

When implanting an expandable prosthetic valve, it is desirable to expand the valve to a maximum size allowed by the patient's anatomical considerations, in order to avoid paravalvular leakage or other unfavorable hemodynamic phenomena across the valve that may be associated with a mismatch between the valve's expansion diameter and the surrounding tissue, while mitigating the risk of annular rupture that may result from over-expansion. To ensure optimal implantation size, the diameter of the prosthetic valve should be monitored in real-time during the implantation procedure. While real-time monitoring may be of importance for all types of prosthetic valves, mechanically expandable may particularly benefit from such monitoring, since mechanical actuation mechanisms provides a higher degree of control over the rate and extent of valve expansion, enabling the clinician to adjust expansion diameter in response to real-time monitored data.

### SUMMARY OF THE INVENTION

The present disclosure is directed toward devices, assemblies and methods for monitoring radial expansion of a prosthetic valve during prosthetic valve implantation procedures. Real-time measurement of the expansion diameter ensures proper implantation of the prosthetic valve within a designated site of implantation, such as the site of malfunctioning native valve.

According to one aspect of the invention, there is provided a delivery assembly comprising a prosthetic valve and a delivery apparatus. The prosthetic valve is movable between a radially compressed configuration and a radially expanded configuration. The delivery apparatus comprises a handle, a delivery shaft extending distally from the handle, and a re-compression assembly. The re-compression assembly comprises a re-compression shaft extending through a lumen of the delivery shaft, and a re-compression member extending through a lumen of the re-compression shaft. The re-compression member comprises a loop portion configured to circumscribe the prosthetic valve, wherein the loop portion comprises at least one radiopaque marker. Relative movement between the re-compression member and the re-compression shaft in the axial direction is effective to tighten the loop portion around the prosthetic valve, thereby radially compressing the prosthetic valve.

According to some embodiments, the at least one radiopaque marker comprises a plurality of radiopaque markers, spaced from each other along at least a portion of the loop portion.

According to some embodiments, the radiopaque markers comprise radiopaque bands.

According to some embodiments, the radiopaque markers span along a portion of the loop portion that is at least as long as half of the prosthetic valve perimeter in the radially expanded configuration.

According to some embodiments, the at least one radiopaque marker is disposed along a minimal marking length, at a position which corresponds to the contact region between the loop portion and the perimeter of the prosthetic valve.

According to some embodiments, the minimal marking length is at least as great as the perimeter of the prosthetic valve in the radially expanded configuration.

According to some embodiments, the at least one radiopaque marker comprises radiopaque coating.

According to some embodiments, the re-compression member further comprises a releasable connector. The releasable connector comprises a proximal connector element and a distal connector element releasably attached to each other, wherein the re-compression member comprises a re-compression member proximal segment coupled to the proximal connector element, and wherein the loop portion is coupled to the distal connector element.

According to some embodiments, the prosthetic valve comprises a guide member, wherein at least a portion of the re-compression member extends through a lumen of the guide member.

According to some embodiments, the prosthetic valve further comprises a sleeve disposed around at least a portion of the circumference of the prosthetic valve, wherein at least a portion of the loop portion extends through the sleeve.

According to another aspect of the invention, there is provided a delivery assembly comprising a prosthetic valve and a delivery apparatus. The prosthetic valve is movable between a radially compressed configuration and a radially expanded configuration. The delivery apparatus comprises a handle, a delivery shaft extending distally from the handle, and a re-compression assembly. The re-compression assembly comprises a re-compression shaft extending through a lumen of the delivery shaft, and a re-compression member comprising at least one indicator radiopaque marker. The re-compression shaft comprises at least one reference radiopaque marker.

The re-compression member comprises a re-compression member proximal segment and loop portion. The re-compression member extends through a lumen of the re-compression shaft. The loop potion extends distally from the re-compression shaft. Relative movement between the re-compression member and the re-compression shaft in the axial direction is effective to tighten the loop portion around the prosthetic valve, thereby radially compressing the prosthetic valve. The axial position of the one indicator radiopaque marker, relative to the at least one reference radiopaque marker, is indicative of the diameter of the prosthetic valve.

According to some embodiments, the at least one reference radiopaque marker comprises a plurality of reference radiopaque markers, wherein each reference radiopaque marker is associated with a different diameter of the prosthetic valve, and wherein alignment of the indicator radiopaque marker with any one of the reference radiopaque markers is indicative of the diameter associated with the respective reference radiopaque marker.

According to some embodiments, the re-compression member proximal segment comprises the at least one indicator radiopaque marker.

According to some embodiments, the re-compression member further comprises a connector, coupled to the re-compression member proximal segment and to the loop portion.

According to some embodiments, the connector comprises the at least one indicator radiopaque marker.

According to some embodiments, the connector is a releasable connector, comprising a proximal connector element and a distal connector element releasably attached to each other, wherein the re-compression member proximal segment is coupled to the proximal connector element, and wherein the loop portion is coupled to the distal connector element.

According to some embodiments, the prosthetic valve comprises a guide member, wherein at least a portion of the re-compression member extends through a lumen of the guide member.

According to some embodiments, the prosthetic valve further comprises a sleeve disposed around at least a portion of the circumference of the prosthetic valve, wherein at least a portion of the loop portion extends through the sleeve.

According to some embodiments, the delivery assembly further comprises a plurality of actuation arm assemblies coupled to the prosthetic valve, and configured to move the prosthetic valve between the radially compressed and the radially expanded configurations. The plurality of actuation arm assemblies comprises a plurality of loop attachment members, wherein the loop portion is coupled to, and extends between, the plurality of loop attachment members.

According to some embodiments, the handle further comprises a spring connected to the re-compression member proximal segment, and configured to apply an axially oriented pull-force on the re-compression member proximal segment, wherein the pull-force is sufficient to apply a minimal tension magnitude to the loop portion.

According to some embodiments, handle further comprises a pulley assembly, comprising a first pulley and a second pulley. The first pulley is attached to the handle via a first pin and rotatable around the first pin. The second pulley is attached to the handle via a second pin and rotatable around the second pin. The re-compression member proximal segment is routed partially around the first pulley and around the second pulley. The pulley assembly is configured to apply a minimal tension magnitude to the loop portion.

According to another aspect of the invention, there is provided a delivery assembly comprising a prosthetic valve and a delivery apparatus. The prosthetic valve is movable between a radially compressed configuration and a radially expanded configuration. The delivery apparatus comprises a handle, a delivery shaft extending distally from the handle, a re-compression assembly and a diameter gauge.

The re-compression assembly comprises a re-compression shaft extending through a lumen of the delivery shaft, and a re-compression member extending through a lumen of the re-compression shaft. The re-compression member comprises a re-compression member proximal segment and a loop portion extending distally from the re-compression shaft. Relative movement between the re-compression member and the re-compression shaft in the axial direction is effective to apply tension to the loop portion, thereby radially compressing the prosthetic valve.

The diameter gauge is coupled to the re-compression assembly at a gauge coupling point, and is configured to provide a real-time indication of the diameter of the prosthetic valve, based on axial position and/or axial translation of the gauge coupling point.

According to some embodiments, the delivery apparatus further comprises a plurality of actuation arm assemblies coupled to the prosthetic valve, and configured to move the prosthetic valve between the radially compressed and the radially expanded configurations. The plurality of actuation arm assemblies further comprise a plurality of loop attachment members. The plurality of actuation arm assemblies comprises a plurality of loop attachment members, wherein the loop portion is coupled to, and extends between, the plurality of loop attachment members.

According to some embodiments, the loop portion is configured to circumscribe the prosthetic valve, such that relative movement between the re-compression member and the re-compression shaft in the axial direction is effective to tighten the loop portion around the prosthetic valve.

According to some embodiments, the handle further comprises a spring connected to the re-compression member proximal segment, and configured to apply an axially oriented pull-force on the re-compression member proximal segment, wherein the pull-force is sufficient to apply a minimal tension magnitude to the loop portion.

According to some embodiments, the handle further comprises a pulley assembly. The pulley assembly comprises a first pulley attached to the handle via a first pin and rotatable around the first pin, and a second pulley attached to the handle via a second pin and rotatable around the second pin. The re-compression member proximal segment is routed partially around the first pulley and around the second pulley. The pulley assembly is configured to apply a minimal tension magnitude to the loop portion.

According to some embodiments, the second pulley further comprises a pole portion and a gear portion, and the handle further comprises a rack. The rack is configured to engage with the gear portion, such that axial translation of the rack is effective to rotate the gear portion. The re-compression member proximal segment is configured to wrap around the pole portion.

According to some embodiments, the handle further comprises a display, wherein the real time indication is a visual real-time indication, visible via the display.

According to some embodiments, the diameter gauge comprises indicator marks, reflecting the range of the diameters of the prosthetic valve, and a dial. The dial is coupled to the re-compression assembly at the gauge coupling point, and configured to point at the indicator mark representing the diameter of the prosthetic valve.

According to some embodiments, the dial is attached to the handle via a dial pivot, and configured to rotate angularly about the dial pivot when the gauge coupling point translates in an axial direction.

According to some embodiments, the dial is orthogonal to a longitudinal axis of the re-compression proximal segment, and configured to move along with the re-compression assembly when the re-compression proximal segment translates in an axial direction.

According to some embodiments, the dial is attached, at the gauge coupling point, to the re-compression member proximal segment.

According to some embodiments, the diameter gauge comprises a displacement sensor, operatively connected to the re-compression assembly, and configured to generate a signal, wherein the magnitude of the signal is proportional to the position and/or axial displacement gauge coupling point.

According to some embodiments, the displacement sensor comprises a potentiometer, and the diameter gauge further comprises a wiper coupled to the re-compression assembly at the gauge coupling point. The wiper is configured to contact the potentiometer at an end of the wiper opposite to the gauge coupling point.

According to some embodiments, the wiper is attached, at the gauge coupling point, to the re-compression member proximal segment.

According to some embodiments, the re-compression assembly further comprises a track member extending through the lumen of the re-compression shaft. The track member comprises a track member proximal segment and a secondary loop extending distally from the re-compression shaft.

According to some embodiments, the dial is attached, at the gauge coupling point, to the track member proximal segment.

According to some embodiments, the wiper attached, at the gauge coupling point, to the track member proximal segment.

According to some embodiments, the plurality of actuation arm assemblies further comprise a plurality of secondary loop attachment members, wherein the secondary loop is coupled to, and extends between, the plurality of secondary loop attachment members.

According to some embodiments, the handle further comprises a track spring connected to the track member proximal segment, and configured to apply an axially oriented pull-force on the track member proximal segment, wherein the pull-force is sufficient to apply a minimal tension magnitude to the secondary loop.

According to another aspect of the invention, there is provided a method of providing an indication of the expansion diameter of a prosthetic valve, comprising the steps: (i) acquiring at least one image of the frame of a prosthetic valve; (ii) deriving a dimensionless parameter from the at least one image; (iii) associating a numerical value of an expansion diameter of the prosthetic valve with the dimensionless parameter, and (iv) providing a visual indication of the expansion diameter of the prosthetic valve.

According to some embodiments, the step of acquiring at least one image comprises acquiring at least one angiogram X-ray image of the frame.

According to some embodiments, the step of acquiring at least one image comprises acquiring at least one fluoroscopy image of the frame.

According to some embodiments, the step of associating a numerical value of an expansion diameter of the prosthetic valve with the dimensionless parameter is based on any of: mathematical formulas, graphs, and/or tables.

According to some embodiments, the step of providing a visual indication comprises visualizing the expansion diameter of the prosthetic valve on a digital screen, as: a numerical value, a graphical symbol, a textual message, or any combination thereof.

According to some embodiments, the dimensionless parameter is an aspect ratio of a length of the frame and a width of the frame.

According to some embodiments, the dimensionless parameter is an opening angle between two intersecting struts of the frame.

According to another aspect of the invention, there is provided a prosthetic valve comprising a frame and a frame belt. The frame is movable between a radially compressed configuration and a radially expanded configuration. The frame belt comprises at least one expansion force indicator. At least a portion of the frame belt extends along at least a portion of the circumference of the frame in the expanded configuration. The at least one expansion force indicator is configured to change a state thereof, when a force exceeding a specific magnitude is applied thereto by the frame, during frame expansion.

According to some embodiments, the at least one expansion force indicator comprises a radiopaque marker, wherein the change in the state of the at least one expansion force indicator is visible under fluoroscopy.

According to some embodiments, the radiodensity of the at least one expansion force indicator is higher than a radiodensity of the frame.

According to some embodiments, the at least one expansion force indicator comprises a separation zone.

According to some embodiments, the separation zone comprises a frangible portion.

According to some embodiments, the frangible portion comprises a plurality of frangible portions, wherein at least two frangible portions are configured to disrupt in response to different tensioning force magnitudes applied thereto.

According to some embodiments, the separation zone comprises a decouplable portion.

According to some embodiments, the frame belt comprises a plurality of expandable portions and a plurality of base portions attached thereto, wherein the at least one separation zone comprises a plurality of separation zone, such that each separation zone is comprised in a respective base portion. The expendable portions are configured to circumferentially expand along with the frame.

According to some embodiments, the separation zone comprises a radiopaque marking, wherein the change in the state of the at least one expansion force indicator comprises a transition of the separation zone from an intact state to a separated state.

According to some embodiments, the expandable portions comprise radiopaque markings, wherein the change in the state of the at least one expansion force indicator comprises a transition of a height of the respective expandable portion from a first height value to a second, shorter height value.

According to some embodiments, the at least one expansion force indicator comprises a geometrical feature, wherein the geometrical feature has a shape which is distinguishable from its neighboring zone along the frame belt, and wherein the change in the state of the at least one expansion force indicator comprises translation of the geometrical feature from a first zone to a second zone.

According to some embodiments, the prosthetic valve further comprises a restrictor configured to allow passage of the at least one geometrical feature there-through, upon application of a pulling force on the frame belt, exceeding a predetermined threshold.

According to some embodiments, the first zone comprises a radiopaque-covered zone, configured to mask the geometrical feature when disposed therein, and the second zone comprises an exposed zone, in which the geometrical feature is visible under fluoroscopy when disposed therein.

According to some embodiments, the first zone comprises a first orientation of a portion of the frame belt, and the second zone comprises a second orientation of a portion of the frame belt, wherein the second orientation is angled relative to the first orientation.

According to some embodiments, the prosthetic valve further comprises a reference radiopaque marker, wherein the first zone comprises a first spatial position of the geometrical feature relative to the reference radiopaque marker, wherein the second zone comprises a second spatial position of the geometrical feature relative to the reference radiopaque marker, and wherein the first spatial position and the second spatial position are on opposite sides of the reference radiopaque marker.

According to some embodiments, prosthetic valve further comprises a sleeve disposed around at least a portion of the circumference of the prosthetic valve, wherein at least a portion of the frame belt extends through the sleeve in at least one configuration of the prosthetic valve.

According to some embodiments, the at least one geometrical feature comprises a bead.

According to some embodiments, the at least one geometrical feature comprises a belt ratcheting tooth.

According to some embodiments, the restrictor comprises an eyelet.

According to some embodiments, the restrictor comprises sleeve ratcheting teeth.

According to some embodiments, the frame belt comprises a bio-resorbable material.

According to some embodiments, there is provided a delivery assembly comprising the prosthetic valve and a delivery apparatus. The delivery apparatus comprises a handle and a belt pull member, wherein the belt pull member extends distally from the handle, and is attached to the frame belt.

According to some embodiments, the delivery assembly further comprises a belt shaft extending distally from the handle, wherein at least a portion of the belt pull member extends through, and is axially movable relative to, the belt shaft.

According to some embodiments, prosthetic valve further comprises a guide member, wherein at least a portion of the frame belt extends through a lumen of the guide member.

According to some embodiments, the delivery assembly further comprises a releasable connector. The releasable connector comprises a proximal connector element and a distal connector element releasably attached to each other, wherein the belt pull member is coupled to the proximal connector element, and wherein frame belt is coupled to the distal connector element.

According to some embodiments, there is provided a delivery assembly comprising the prosthetic valve and a delivery apparatus. The delivery apparatus comprises a handle and a transmission line. The transmission line extends distally from the handle, and is coupled to the frame belt. The at least one expansion force indicator comprises a stretch sensor, wherein the change in the state of the stretch sensor comprises a change of an electrical property thereof, when stretched over the prosthetic valve. The transmission line is configured to conduct electric signals from the stretch sensor toward the handle.

Certain embodiments of the present invention may include some, all, or none of the above advantages. Further advantages may be readily apparent to those skilled in the art from the figures, descriptions, and claims included herein. Aspects and embodiments of the invention are further described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following embodiments and aspects thereof are described and illustrated in conjunction with systems, tools and methods which are meant to be exemplary and illustrative, but not limiting in scope. In various embodiments, one or more of the above-described problems have been reduced or eliminated, while other embodiments are directed to other advantages or improvements.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

In the Figures:

FIG. 1 shows a view in perspective of a delivery assembly comprising a delivery apparatus carrying a prosthetic valve, according to some embodiments.

FIG. 2 shows a view in perspective of a prosthetic valve, according to some embodiments.

FIG. 3A shows a view in perspective of an inner member, according to some embodiments.

FIG. 3B shows a view in perspective of an actuator assembly, according to some embodiments.

FIG. 3C shows a view in perspective of a prosthetic valve including multiple actuator assemblies of the type shown in FIG. 3B.

FIGS. 4A-4C show an actuator assembly of the type shown in FIG. 3B in different operational states thereof.

FIGS. 5A-5C show different stages of utilizing a delivery assembly equipped with a re-compression assembly, according to some embodiments.

FIG. 6A shows a delivery assembly equipped with a re-compression assembly having a plurality of radiopaque markers, according to some embodiments.

FIG. 6B shows a delivery assembly equipped with a re-compression assembly having a single continuous radiopaque marker, according to some embodiments.

FIGS. 7A-7C show different stages of utilizing a delivery assembly, equipped with a re-compression assembly having a releasable connector, according to some embodiments.

FIG. 8A shows a delivery assembly equipped with a re-compression assembly having a distal segment extending between actuation arm assemblies and a proximal segment coupled to a dial of a diameter gauge, in a compressed state of the prosthetic valve, according to some embodiments.

FIG. 8B shows the delivery assembly of FIG. 8A, in an expanded state of the prosthetic valve.

FIG. 8C shows a delivery assembly equipped with a re-compression assembly having a distal segment circumscribing the prosthetic valve and a proximal segment coupled to a dial of a diameter gauge, in a compressed state of the prosthetic valve, according to some embodiments.

FIG. 8D shows the delivery assembly of FIG. 8C, in an expanded state of the prosthetic valve.

FIG. 9 shows a delivery assembly equipped with a re-compression assembly coupled to a non-pivotable dial of a diameter gauge, according to some embodiments.

FIG. 10 shows a delivery assembly equipped with a re-compression assembly coupled to a dial of a diameter gauge, and routed through a pulley assembly, according to some embodiments.

FIG. 11 shows a delivery assembly with a re-compression assembly coupled to a displacement sensor of a diameter gauge, according to some embodiments.

FIG. 12 shows a delivery assembly with a track member of a re-compression assembly coupled to a diameter gauge, according to some embodiments.

FIGS. 13A-13B show different states a delivery assembly, equipped with a re-compression assembly having indicator and reference markers, according to some embodiments.

FIGS. 14A-14B show different states a delivery assembly, equipped with a re-compression assembly having indicator and reference markers, according to additional embodiments.

FIGS. 15A-15B show different states a delivery assembly, equipped with a re-compression assembly having indicator and reference markers, according to additional embodiments.

FIGS. 16A-16B show different states a delivery assembly, equipped with a re-compression assembly having indicator and reference markers, according to additional embodiments.

FIG. 17 shows zoomed-in view of a portion of a re-compression assembly, having a plurality of reference markers and a plurality of indicator markers, according to some embodiments.

FIG. 18 shows a delivery assembly with a track member of a re-compression assembly, provided with indicator and reference markers, according to some embodiments.

FIGS. 19A and 19B show a prosthetic valve having a length and diameter varying between a crimped and an expanded state, respectively, according to some embodiments.

FIG. 20 shows a curve representing the relationship between the aspect ratio and the expansion diameter of a prosthetic valve, according to some embodiments.

FIGS. 21A and 21B show a prosthetic valve having opening angles varying between a crimped and an expanded state, respectively, according to some embodiments.

FIG. 22 shows a curve representing the relationship between the opening angle and the expansion diameter of a prosthetic valve, according to some embodiments.

FIGS. 23A-23C show different states a prosthetic valve provided with a frame belt, according to some embodiments.

FIGS. 24A-24B show different states a portion of a frame belt provided with frangible portions, according to some embodiments.

FIGS. 25A-25B show different states a portion of a frame belt provided with a decouplable portion, according to some embodiments.

FIGS. 26A-26D show different stages of utilizing a delivery assembly equipped with a frame belt having a plurality of geometrical features, according to some embodiments.

FIG. 27 shows a prosthetic valve equipped with a frame belt extending through a guide member restriction, according to some embodiments.

FIGS. 28A-28B show different states a prosthetic valve provided with a beaded frame belt disposed there-around, according to some embodiments.

FIGS. 29A-29B show different states a prosthetic valve provided with a ratcheting frame belt disposed there-around, according to some embodiments.

### DETAILED DESCRIPTION OF SOME EMBODIMENTS

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout.

Throughout the figures of the drawings, different superscripts for the same reference numerals are used to denote different embodiments of the same elements. Embodiments of the disclosed devices and systems may include any combination of different embodiments of the same elements. Specifically, any reference to an element without a superscript may refer to any alternative embodiment of the same element denoted with a superscript.

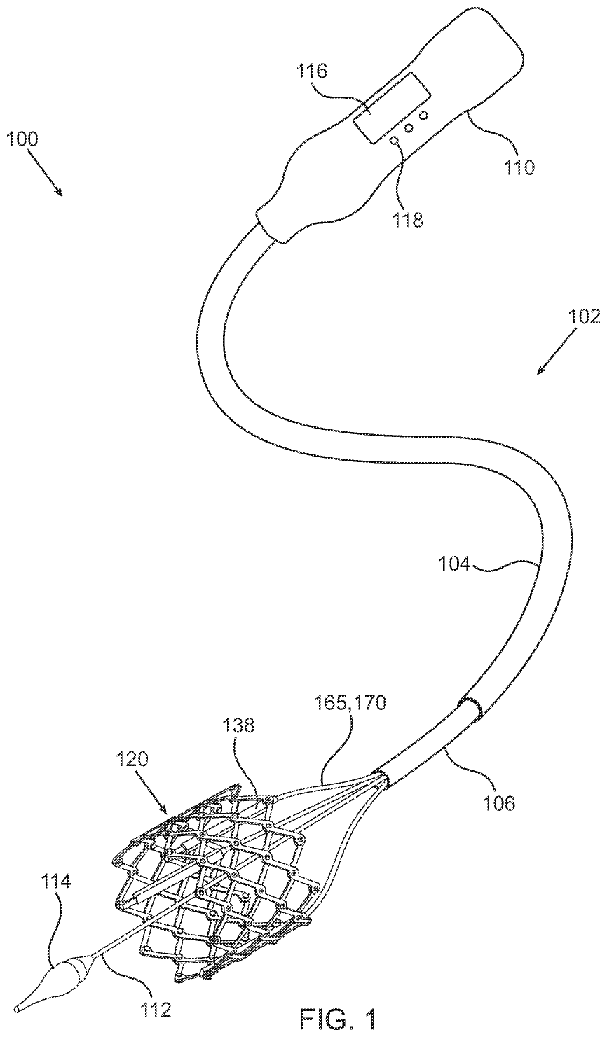

FIG. 1 constitutes a view in perspective of a delivery assembly 100, according to some embodiments. The delivery assembly 100 can include a prosthetic valve 120 and a delivery apparatus 102. The prosthetic valve 120 can be on or releasably coupled to the delivery apparatus 102. The delivery apparatus can include a handle 110 at a proximal end thereof, a nose cone shaft 112 extending distally from the handle 110, a nose cone 114 attached to the distal end of the nosecone shaft 112, a delivery shaft 106 extending over the nose cone shaft 112, and optionally an outer shaft 104 extending over the delivery shaft 106.

The term “proximal”, as used herein, generally refers to the side or end of any device or a component of a device, which is closer to the handle 110 or an operator of the handle 110 when in use.

The term “distal”, as used herein, generally refers to the side or end of any device or a component of a device, which is farther from the handle 110 or an operator of the handle 110 when in use.

The term “prosthetic valve”, as used herein, refers to any type of a prosthetic valve deliverable to a patient's target site over a catheter, which is radially expandable and compressible between a radially compressed, or crimped, state, and a radially expanded state. Thus, a prosthetic valve 120 can be crimped or retained by a delivery apparatus 102 in a compressed state during delivery, and then expanded to the expanded state once the prosthetic valve 120 reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

The term “plurality”, as used herein, means more than one.

A prosthetic valve 120 of the current disclosure may include any prosthetic valve configured to be mounted within the native aortic valve, the native mitral valve, the native pulmonary valve, and the native tricuspid valve. While a delivery assembly 100 described in the current disclosure, includes a delivery apparatus 102 and a prosthetic valve 120, it should be understood that the delivery apparatus 102 according to any embodiment of the current disclosure can be used for implantation of other prosthetic devices aside from prosthetic valves, such as stents or grafts.

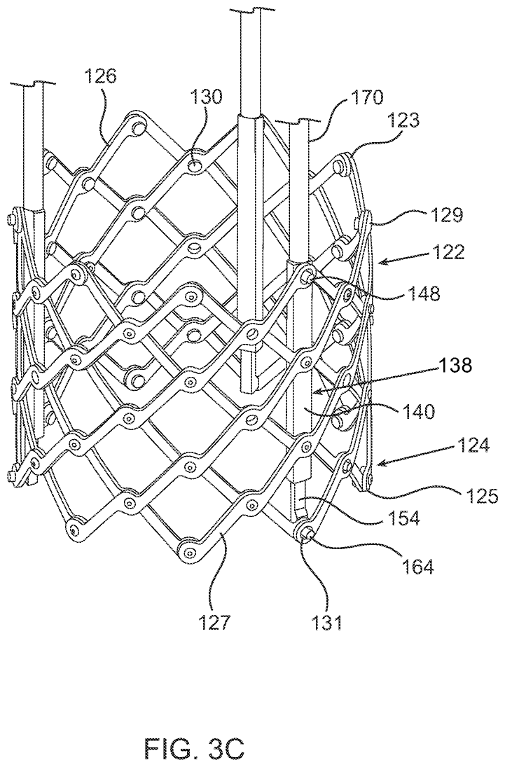

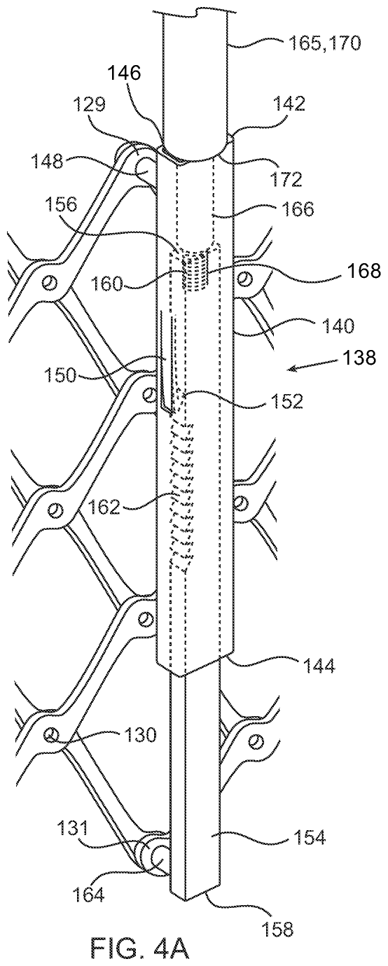

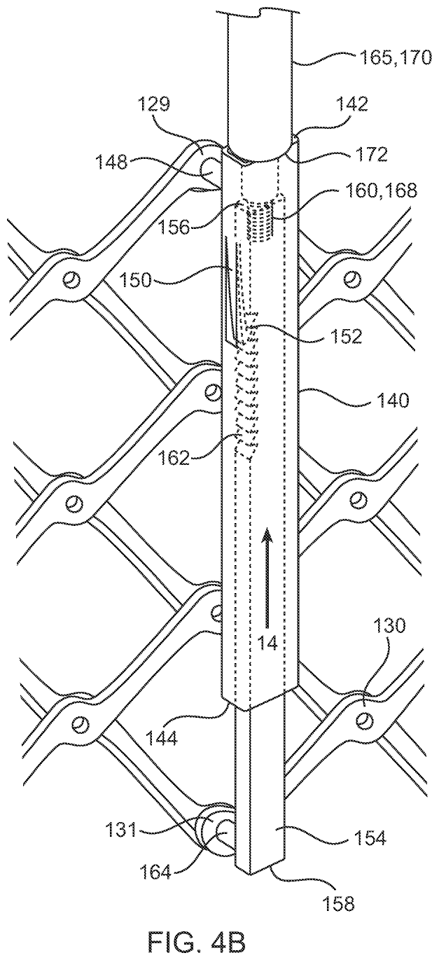

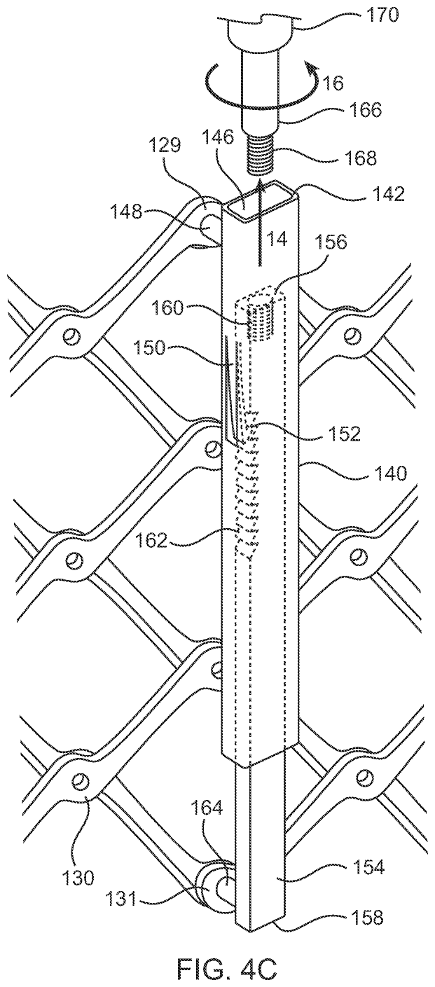

According to some embodiments, the prosthetic valve 120 is a mechanically expandable valve, and the delivery apparatus 102 further comprises a plurality of actuation arm assemblies extending from the handle 110 through the delivery shaft 106. The actuation arm assemblies 165 can generally include actuation members 166 (hidden from view in FIG. 1, visible in FIGS. 4A-4C) releasably coupled at their distal ends to respective actuator assemblies 138 of the valve 120, and support sleeves 170 (annotated in FIG. 3) disposed around the respective actuation members 166. Each actuation member 166 may be axially movable relative to the support sleeve 170 covering it.

The prosthetic valve 120 can be delivered to the site of implantation via a delivery assembly 100 carrying the valve 120 in a radially compressed or crimped state, toward the target site, to be mounted against the native anatomy, by expanding the valve 120 via a mechanical expansion mechanism, as will be elaborated below.

The delivery assembly 100 can be utilized, for example, to deliver a prosthetic aortic valve for mounting against the aortic annulus, to deliver a prosthetic mitral valve for mounting against the mitral annulus, or to deliver a prosthetic valve for mounting against any other native annulus.

The nosecone 114 can be connected to the distal end of the nosecone shaft 112. A guidewire (not shown) can extend through a central lumen of the nosecone shaft 112 and an inner lumen of the nosecone 114, so that the delivery apparatus 102 can be advanced over the guidewire through the patient's vasculature.

A distal end portion of the outer shaft 104 can extend over the prosthetic valve 120 and contact the nosecone 114 in a delivery configuration of the delivery apparatus 102. Thus, the distal end portion of the outer shaft 104 can serve as a delivery capsule that contains, or houses, the prosthetic valve 120 in a radially compressed or crimped configuration for delivery through the patient's vasculature.

The outer shaft 104 and the delivery shaft 106 can be configured to be axially movable relative to each other, such that a proximally oriented movement of the outer shaft 104 relative to the delivery shaft 106, or a distally oriented movement of the delivery shaft 106 relative to the outer shaft 104, can expose the prosthetic valve from the outer shaft 104. In alternative embodiments, the prosthetic valve 120 is not housed within the outer shaft 104 during delivery. Thus, according to some embodiments, the delivery apparatus 102 does not include an outer shaft 104.

As mentioned above, the proximal ends of the nose cone shaft 112, the delivery shaft 106, components of the actuation arm assemblies 165, and when present—the outer shaft 104, can be coupled to the handle 110. During delivery of the prosthetic valve 120, the handle 110 can be maneuvered by an operator (e.g., a clinician or a surgeon) to axially advance or retract components of the delivery apparatus 102, such as the nosecone shaft 112, the delivery shaft 106, and/or the outer shaft 104, through the patient's vasculature, as well as to expand or contract the prosthetic valve 120, for example by maneuvering the actuation arm assemblies 165, and to disconnect the prosthetic valve 120 from the delivery apparatus 102, for example—by decoupling the actuation members 166 from the actuator assemblies 138 of the valve 120, in order to retract it once the prosthetic valve is mounted in the implantation site.

The term “and/or” is inclusive here, meaning “and” as well as “or”. For example, “delivery shaft 106 and/or outer shaft 104” encompasses, delivery shaft 106, outer shaft 104, and delivery shaft 106 with outer shaft 104; and, such “delivery shaft 106 and/or outer shaft 104” may include other elements as well.

According to some embodiments, the handle 110 can include one or more operating interfaces, such as steerable or rotatable adjustment knobs, levers, sliders, buttons (not shown) and other actuating mechanisms, which are operatively connected to different components of the delivery apparatus 102 and configured to produce axial movement of the delivery apparatus 102 in the proximal and distal directions, as well as to expand or contract the prosthetic valve 120 via various adjustment and activation mechanisms as will be further described below.

According to some embodiments, the handle further comprises one or more visual or auditory informative elements configured to provide visual or auditory information and/or feedback to a user or operator of the delivery apparatus 102, such as a display 116, LED lights 118, speakers (not shown) and the like.

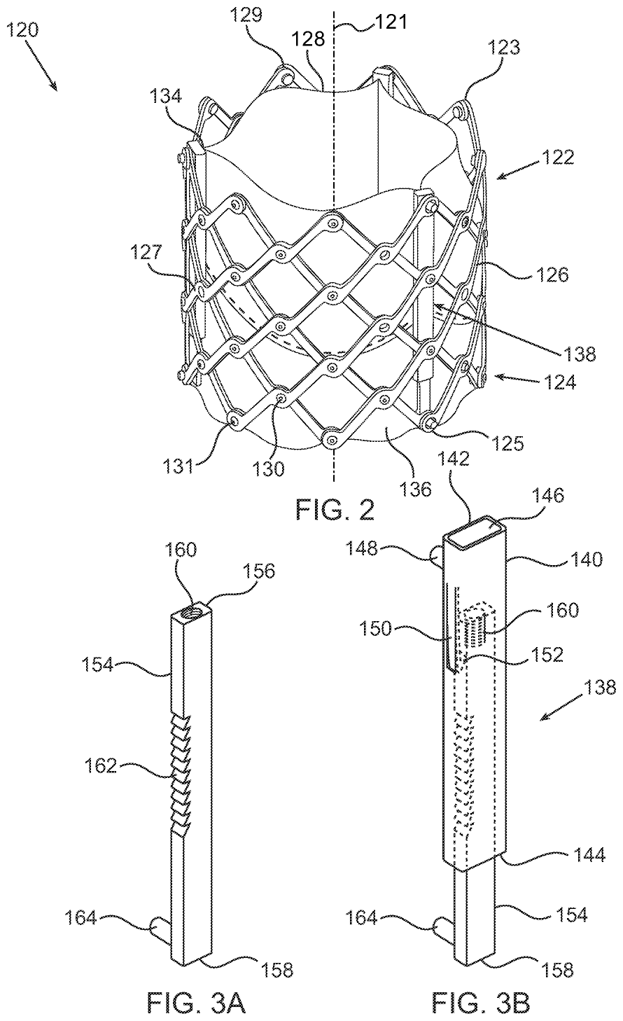

FIG. 2 shows an exemplary mechanically expandable prosthetic valve 120 in an expanded state, according to some embodiments. The prosthetic valve 120 can comprise an inflow end portion 124 defining an inflow end 125, and an outflow end portion 122 defining an outflow end 123. The prosthetic valve 120 can define a longitudinal axis 121 extending through the inflow end portion 124 and the outflow end portion 122. In some instances, the outflow end 123 is the distal end of the prosthetic valve 120, and the inflow end 125 is the proximal end of the prosthetic valve 120. Alternatively, depending for example on the delivery approach of the valve, the outflow end can be the proximal end of the prosthetic valve, and the inflow end can be the distal end of the prosthetic valve.

The term “outflow”, as used herein, refers to a region of the prosthetic valve through which the blood flows through and out of the valve 120, for example between the longitudinal axis 121 and the outflow end 123.

The term “inflow”, as used herein, refers to a region of the prosthetic valve through which the blood flows into the valve 120, for example between inflow end 125 and the longitudinal axis 121.

The valve 120 comprises a frame 126 composed of interconnected struts 127, and may be made of various suitable materials, such as stainless steel, cobalt-chrome alloy (e.g. MP35N alloy), or nickel titanium alloy such as Nitinol. According to some embodiments, the struts 127 are arranged in a lattice-type pattern. In the embodiment illustrated in FIG. 2, the struts 127 are positioned diagonally, or offset at an angle relative to, and radially offset from, the longitudinal axis 121 when the valve 120 is in an expanded position. It will be clear that the struts 127 can be offset by other angles than those shown in FIG. 2, such as being oriented substantially parallel to the longitudinal axis 121.

According to some embodiments, the struts 127 are pivotably coupled to each other. In the exemplary embodiment shown in FIG. 2, the end portions of the struts 127 are forming apices 129 at the outflow end 123 and apices 131 at the inflow end 125. The struts 127 can be coupled to each other at additional junctions 130 formed between the outflow apices 129 and the inflow apices 131. The junctions 130 can be equally spaced apart from each other, and/or from the apices 129, 131 along the length of each strut 127. Frame 126 may comprise openings or apertures at the regions of apices 129, 131 and junctions 130 of the struts 127. Respective hinges can be included at locations where the apertures of struts 127 overlap each other, via fasteners, such as rivets or pins, which extend through the apertures. The hinges can allow the struts 127 to pivot relative to one another as the frame 126 is radially expanded or compressed.

In alternative embodiments, the struts are not coupled to each other via respective hinges, but are otherwise pivotable or bendable relative to each other, so as to permit frame expansion or compression. For example, the frame can be formed from a single piece of material, such as a metal tube, via various processes such as, but not limited to, laser cutting, electroforming, and/or physical vapor deposition, while retaining the ability to collapse/expand radially in the absence of hinges and like.

A prosthetic valve 120 further comprises one or more leaflets 128, e.g., three leaflets, configured to regulate blood flow through the prosthetic valve 120 from the inflow end to the outflow end. While three leaflets 128 arranged to collapse in a tricuspid arrangement, are shown in the exemplary embodiment illustrated in FIG. 2, it will be clear that a prosthetic valve 120 can include any other number of leaflets 128. The leaflets 128 are made of a flexible material, derived from biological materials (e.g., bovine pericardium or pericardium from other sources), bio-compatible synthetic materials, or other suitable materials. The leaflets may be coupled to the frame 126 via commissures 134, either directly or attached to other structural elements connected to the frame 126 or embedded therein, such as commissure posts. Further details regarding prosthetic valves, including the manner in which leaflets may be mounted to their frames, are described in U.S. Pat. Nos. 6,730,118, 7,393,360, 7,510,575, 7,993,394 and 8,252,202, and U.S. Patent Application No. 62/614,299, all of which are incorporated herein by reference.

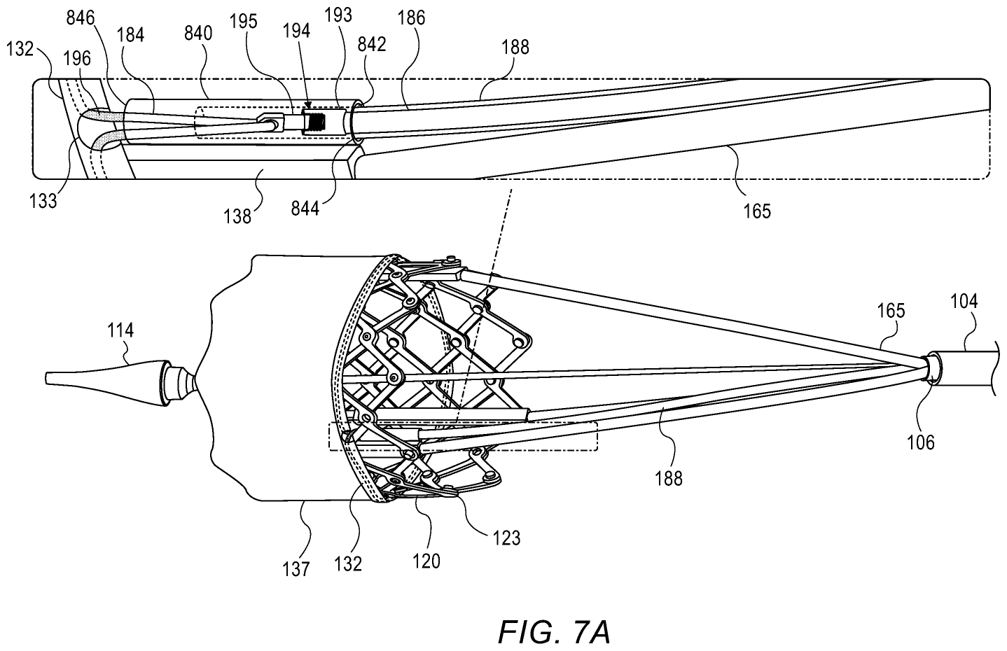

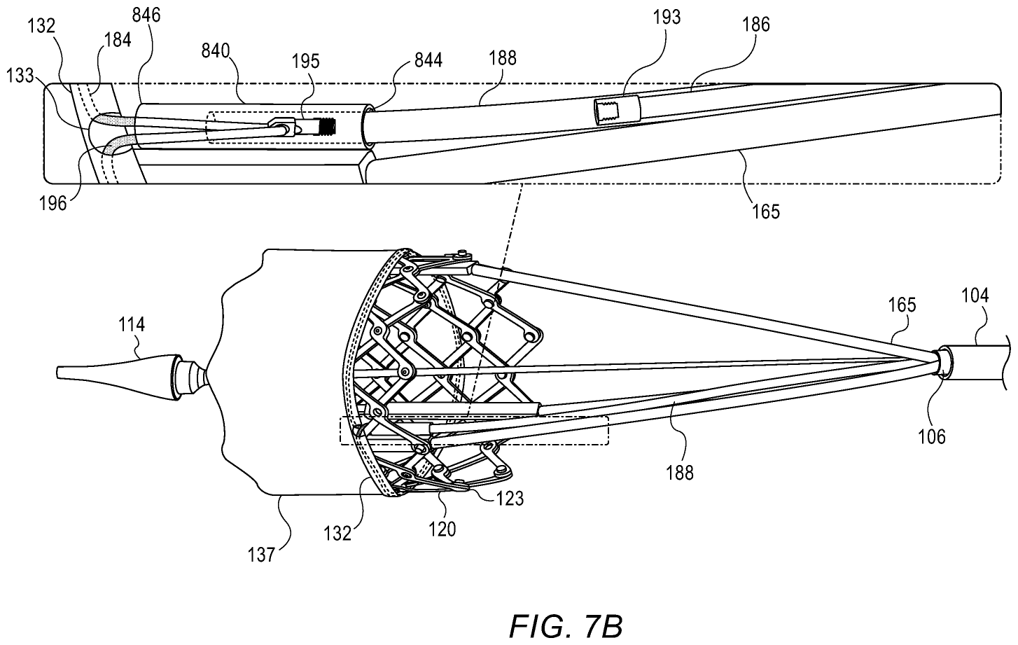

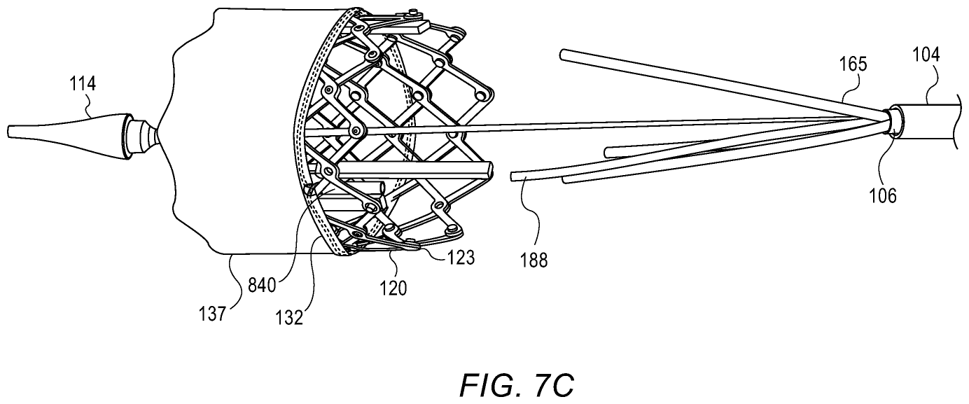

According to some embodiments, the prosthetic valve 120 may further comprise at least one skirt or sealing member, such as the inner skirt 136 shown in the exemplary embodiment illustrated in FIG. 2. The inner skirt 136 can be mounted on the inner surface of the frame 126, configured to function, for example, as a sealing member to prevent or decrease perivalvular leakage. The inner skirt 136 can further function as an anchoring region for the leaflets 128 to the frame 126, and/or function to protect the leaflets 128 against damage which may be caused by contact with the frame 126, for example during valve crimping or during working cycles of the prosthetic valve 120. Additionally, or alternatively, the prosthetic valve 120 can comprise an outer skirt 137 (shown for example in FIGS. 7A-7C) mounted on the outer surface of the frame 126, configure to function, for example, as a sealing member retained between the frame 126 and the surrounding tissue of the native annulus against which the prosthetic valve 120 is mounted, thereby reducing risk of paravalvular leakage past the prosthetic valve 120. Any of the inner skirt 136 and/or outer skirt 137 can be made of various suitable biocompatible materials, such as, but not limited to, various synthetic materials (e.g., PET) or natural tissue (e.g. pericardial tissue).

According to some embodiments, a prosthetic valve 120, which can be a mechanical prosthetic valve 120, comprises a plurality of actuator assemblies 138, configured to facilitate expansion of the valve 120, and in some instances, to lock the valve at an expanded state, preventing unintentional recompression thereof, as will be further elaborated below. Although FIG. 2 illustrates three actuator assemblies 138, mounted to, and equally spaced, around an inner surface of the frame 126, it should be clear that a different number of actuator assemblies 138 may be utilized, that the actuator assemblies 138 can be mounted to the frame 126 around its outer surface, and that the circumferential spacing between actuator assemblies 138 can be unequal.

FIGS. 3A-3B show an exemplary embodiment of an actuator assembly 138. An actuator assembly 138 may include a hollow outer member 140, secured to a component of the valve 120, such as the frame 126, at a first location, and an inner member 154 secured to a component of the valve 120, such as the frame 126, at a second location, axially spaced from the first location.

FIG. 3A constitutes a view in perspective of an exemplary inner member 154, having an inner member proximal end 156 and an inner member distal end 158. The inner member 154 comprises an inner member coupling extension 164 proximate to its distal end 158, which may be formed as a pin extending radially outward from the inner member 154, configured to be received within respective openings or apertures of struts 127 intersecting at a junction 130 or an apex 129, 131. The inner member 154 may further comprise a linear rack having a plurality of teeth 162 along at least a portion of its length. According to some embodiments, one surface of the inner member 154 comprises a plurality of teeth 162.

The terms “including” and/or “having”, as used herein (including the specification and the claims), are defined as comprising (i.e., open language).

FIG. 3B shows the actuation inner member 154 disposed within a lumen 146 of the outer member 140. The outer member 140 is shown with partial transparency in FIG. 3B for sake of clarity. The outer member 140 comprises an outer member proximal end 142 defining a proximal opening, and an outer member distal end 144 defining a distal opening. The outer member 140 can further comprise an outer member coupling extension 148 proximate to its proximal end 142, which may be formed as a pin extending radially outward from the external surface of the outer member 140, configured to be received within respective openings or apertures of struts 127 intersecting at a junction 130 or an apex 129, 131.

The outer member 140 may further comprise a spring biased arm 150, attached to or extending from one sidewall of the outer member 140 and having a tooth or pawl 152 at its opposite end, biased inwards toward the actuation inner member 154 when disposed within the outer member lumen 146.

At least one of the inner or outer member 154 or 140, respectively, is axially movable relative to its counterpart. The actuator assembly 138 in the illustrated embodiments, comprises a ratchet mechanism or a ratchet assembly, wherein the pawl 152 of the outer member 140 is configured to engage with the teeth 162 of the inner member 154. The pawl 152 can have a shape that is complementary to the shape of the teeth 162, such that the pawl 152 allows a sliding movement of the inner member 154 in one direction relative to the outer member 140, for example in a proximally oriented direction, and resists sliding movement of the inner member 154 in the opposite direction, such as a distally oriented direction, when the pawl 152 is in engagement with the teeth 162 of the inner member 154.

The arm 150 can be formed of a flexible or resilient portion of the outer member 140 that extends over and contact, at pawl 152, an opposing side of the outer surface of the inner member 154. According to some embodiments, the arm 150 can be in the form of a leaf spring that can be integrally formed with the outer member 140 or separately formed and subsequently connected to the outer member 140. The arm 150 is configured to apply a biasing force against the outer surface of the inner member 154, so as to ensure that under normal operation, the pawl 152 stays engaged with the teeth 162 of the inner member 154.

According to some embodiments, the inner member 154 further comprises an inner member threaded bore 160 extending from its proximal end 156, configured to receive and threadedly engage with a threaded portion 168 (shown for example in FIGS. 4B-4C) of a corresponding actuation member 166. FIG. 3C shows a view in perspective of a valve 120 in an expanded state, having its actuator assemblies 138 connected to actuation members 166 (hidden from view within the support sleeves 170) of the delivery apparatus 102. The leaflets 128 and skirt 136 are omitted from FIG. 3C to expose the actuator assemblies 138 attached to the frame 126. When actuation members 166 are threaded into the inner members 154, axial movement of the actuation members 166 causes axial movement of the inner members 154 in the same direction.

According to some embodiments, the actuation arm assemblies 165 are configured to releasably couple to the prosthetic valve 120, and to move the prosthetic valve 120 between the radially compressed and the radially expanded configurations. FIGS. 4A-4C illustrate a non-binding configuration representing actuation of the actuator assemblies 138 via the actuation arm assemblies 165 to expand the prosthetic valve 120 from a radially compressed state to a radially expanded state. FIG. 4A shows an actuator assembly 138, having an outer member 140, secured to the frame 126 at a first location, and an inner member 154 secured to the frame 126 at a second location. According to some embodiments, the first location can be positioned at an outflow end portion 122, and the second location can be positioned at the inflow end portion 124. In the illustrated embodiment, the outer member 140 is secured to an outflow apex 129 via outer member coupling extension 148, and the inner member 154 is secured to an inflow apex 131 via inner member coupling extension 164. A proximal portion of the inner member 154 extends, through the distal opening of the outer member distal end 144, into the outer member lumen 146.

The actuator assembly 138 is shown in FIG. 4A in a radially compressed state of the frame valve 120, wherein the outflow and inflow apices 129 and 131, respectively, are relatively distanced apart from each other along the axial direction, and the inner member proximal end 156 is positioned distal to the outer member proximal end 142.

As further shown in FIG. 4A, the distal portion 168 of the actuation member 166 is threadedly engaged with the proximal threaded bore 160 at the proximal end 156 of the inner member 154. According to some embodiments, as shown in FIGS. 4A-4C, the distal portion 168 of the actuation member 166 includes external threads, configured to engage with internal threads of the proximal bore 160 of the inner member 154. According to alternative embodiments, an inner member may include a proximal extension provided with external threads, configured to be received in and engage with internal threads of a distal bore formed within the actuation member (embodiments not shown).

The support sleeve 170 surrounds the actuation member 166 and may be connected to the handle 110. The support sleeve 170 and the outer member 140 are sized such that the distal lip 172 of the support sleeve 170 can abut or engage the outer member proximal end 142, such that the outer member 140 is prevented from moving proximally beyond the support sleeve 170.

In order to radially expand the frame 126, and therefore the valve 120, the support sleeve 170 can be held firmly against the outer member 140. The actuation member 166 can then be pulled in a proximally oriented direction 14, as shown in FIG. 4B. Because the support sleeve 170 is being held against the outer member 140, which is connected to an outflow apex 129, the outflow end 123 of the frame 126 is prevented from moving relative to the support sleeve 170. As such, movement of the actuation member 166 in a proximally oriented direction 14 can cause movement of the inner member 154 in the same direction, thereby causing the frame 126 to foreshorten axially and expand radially.

More specifically, as shown for example in FIG. 4B, the inner member coupling extension 164 extends through openings in two struts 127 interconnected at an inflow apex 131, while the outer member coupling extension 148 extends through openings in two struts 127 interconnected at an outflow apex 129. As such, when the inner member 154 is moved axially, for example in a proximally oriented direction 14, within the outer member 140, the inner member coupling extension 164 moves along with the inner member 154, thereby causing the portion to which the inner member coupling extension 164 is attached to move axially as well, which in turn causes the frame 126 to foreshorten axially and expand radially.

The struts 127 to which the inner member coupling extension 164 is connected are free to pivot relative to the coupling extension 164 and to one another as the frame is expanded or compressed. In this manner, the inner member coupling extension 164 serves as a fastener that forms a pivotable connection between those struts 127. Similarly, struts 127 to which the outer member coupling extension 148 is connected are also free to pivot relative to the coupling extension 148 and to one another as the frame is expanded or compressed. In this manner, the outer member coupling extension 148 also serves as a fastener that forms a pivotable connection between those struts 127.

When the pawl 152 is engaged with the teeth 162, the inner member 154 can move in one axial direction, such as the proximally oriented direction 14, but cannot move in the opposite axial direction. This ensures that while the pawl 152 is engaged with the teeth 162, the frame 126 can radially expand but cannot be radially compressed. Thus, after the prosthetic valve 120 is implanted in the patient, the frame 126 can be expanded to a desired diameter by pulling the actuation member 166. In this manner, the actuation mechanism also serves as a locking mechanism of the prosthetic valve 120.

Once the desired diameter of the prosthetic valve 120 is reached, the actuation member 166 may be rotated in direction 16 to unscrew the actuation member 166 from the inner member 154, as shown in FIG. 4C. This rotation serves to disengage between the distal threaded portion 168 of the actuation member 166 and the inner member threaded bore 160, enabling the actuation arm assemblies 165 to be pulled away, and retracted, together with the delivery apparatus 102, from the patient's body, leaving the prosthetic valve 120 implanted in the patient. The patient's native anatomy, such as the native aortic annulus in the case of transcatheter aortic valve implantation, may exert radial forces against the prosthetic valve 120 that would strive to compress it. However, the engagement between the pawl 152 and the teeth 162 of the inner member 154 prevents such forces from compressing the frame 126, thereby ensuring that the frame 126 remains locked in the desired radially expanded state.

Thus, the prosthetic valve 120 is radially expandable from the radially compressed state shown in FIG. 4A to the radially expanded state shown in FIG. 4B upon actuating the actuator assemblies 138, wherein such actuation includes approximating the second locations to the first locations of the valve. The prosthetic valve 120 is further releasable from the delivery apparatus 102 by decoupling each of the actuation arm assemblies 165 from each corresponding actuator assemblies 138 that was attached thereto.

While the inner member 154 and the outer member 140 are shown in the illustrated embodiment connected to an inflow apex 131 and an outflow apex 129, respectively, it should be understood that they can be connected to other junctions 130 of the frame 126. For example, the inner member coupling extension 164 can extend through openings formed in interconnected struts at a junction 130 at the inflow end portion 124, proximal to the inflow apices 131. Similarly, the outer member coupling extension 148 can extend through openings formed in interconnected struts at a junction 130 at the outflow end portion 122, distal to the outflow apices 129.

While the frame is shown above to expand radially outward by axially moving the inner member 154 in a proximally oriented direction, relative to the outer member 140, it will be understood that similar frame expansion may be achieved by axially pushing an outer member 140 in a distally oriented direction, relative to an inner member 154. Moreover, while the illustrated embodiments show the outer member 140 affixed to an outflow end portion 122 of the frame 126, and an inner member 154 affixed to an inflow end portion 124 of the frame 126, in alternative embodiments, the outer member 140 may be affixed to the inflow end portion 124 of the frame 126, while the inner member 154 may be affixed to the outflow end portion 122 of the frame 126.

According to some embodiments, the handle 110 can comprise control mechanisms which may include steerable or rotatable knobs, levers, buttons and such, which are manually controllable by an operator to produce axial and/or rotatable movement of different components of the delivery apparatus 102. For example, the handle 110 may comprise one or more manual control knobs, such as a manually rotatable control knob that is effective to pull the actuation members 166 when rotated by the operator.

According to other embodiments, control mechanisms in handle 110 and/or other components of the delivery apparatus 102 can be electrically, pneumatically and/or hydraulically controlled. According to some embodiments, the handle 110 can house one or more electric motors which can be actuated by an operator, such as by pressing a button or switch on the handle 110, to produce movement of components of the delivery apparatus 102. For example, the handle 110 may include one or more motors operable to produce linear movement of components of the actuation arm assemblies 165, and/or one or more motors operable to produce rotational movement of the actuation members 166 to disconnect the actuator member distal threaded portion 168 from the actuation inner member threaded bore 160. According to some embodiments, one or more manual or electric control mechanism is configured to produce simultaneous linear and/or rotational movement of all of the actuation members 166.

While a specific actuation mechanism is described above, utilizing a ratcheting mechanism between the inner and the outer members of the actuation assemblies 138, other mechanisms may be employed to promote relative movement between inner and outer members of actuation assemblies, for example via threaded or other engagement mechanisms. Further details regarding the structure and operation of mechanically expandable valves and delivery system thereof are described in U.S. Pat. No. 9,827,093, U.S. Patent Application Publication Nos. 2019/0060057, 2018/0153689 and 2018/0344456, and US Patent Application Nos. 62/870,372 and 62/776,348, all of which are incorporated herein by reference.

Prior to implantation, the prosthetic valve 120 can be crimped onto the delivery apparatus 102. This step can include placement of the radially compressed valve 120 within the outer shaft 104. Once delivered to the site of implantation, such as a native annulus, the valve 120 can be radially expanded within the annulus, for example, by actuating the actuator assemblies 138 described herein above. However, during such implantation procedures, it may become desirable to re-compress the prosthetic valve 120 in situ in order to reposition it. Valve recompression may be achievable, for example, if the mechanical valve 120 has not yet reached a locked state, for example by providing a sufficient smooth length (i.e., devoid of ratcheting teeth 162) along the actuator inner member 154, so as to allow axial movement along a specific distance prior to pawl 152 engagement with the teeth 162. Alternatively or additionally, the delivery assembly 100 can further include release members (not shown), configured to release the pawl 152 from the teeth 162 to allow reversible movement that will enable valve compression.

According to some embodiments, the delivery apparatus 102 further comprises a re-compression assembly 180, configured to facilitate re-compression of a prosthetic valve 120 upon expansion thereof.

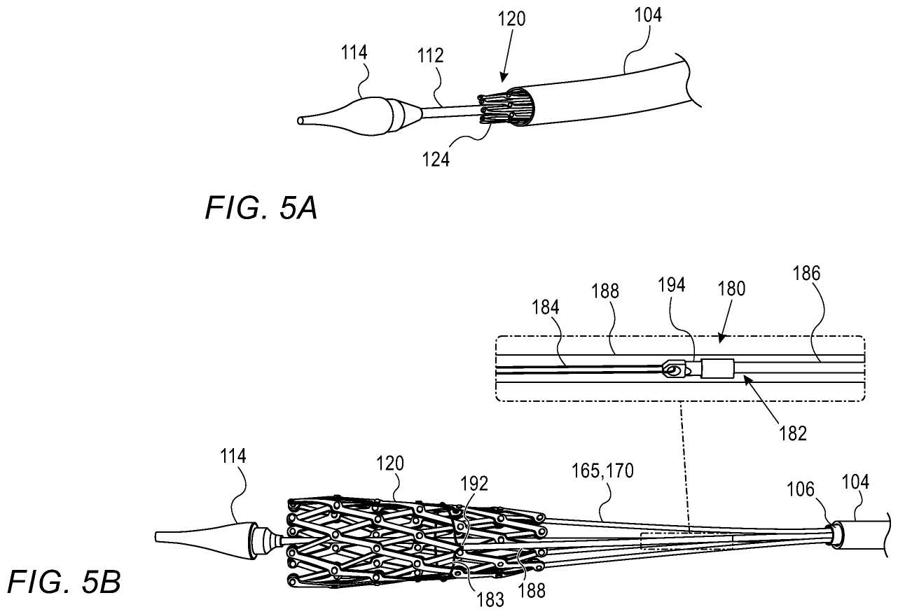

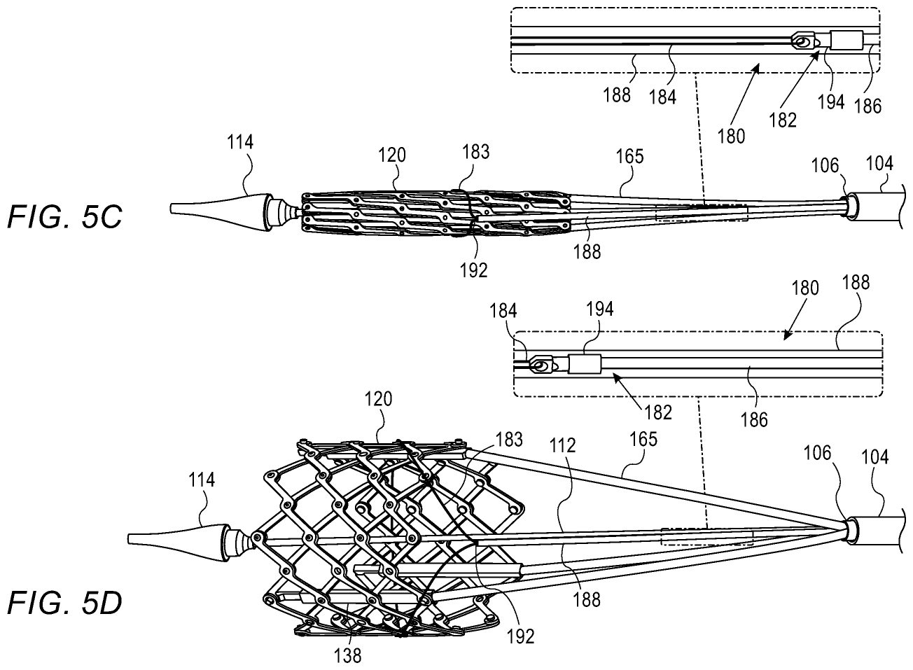

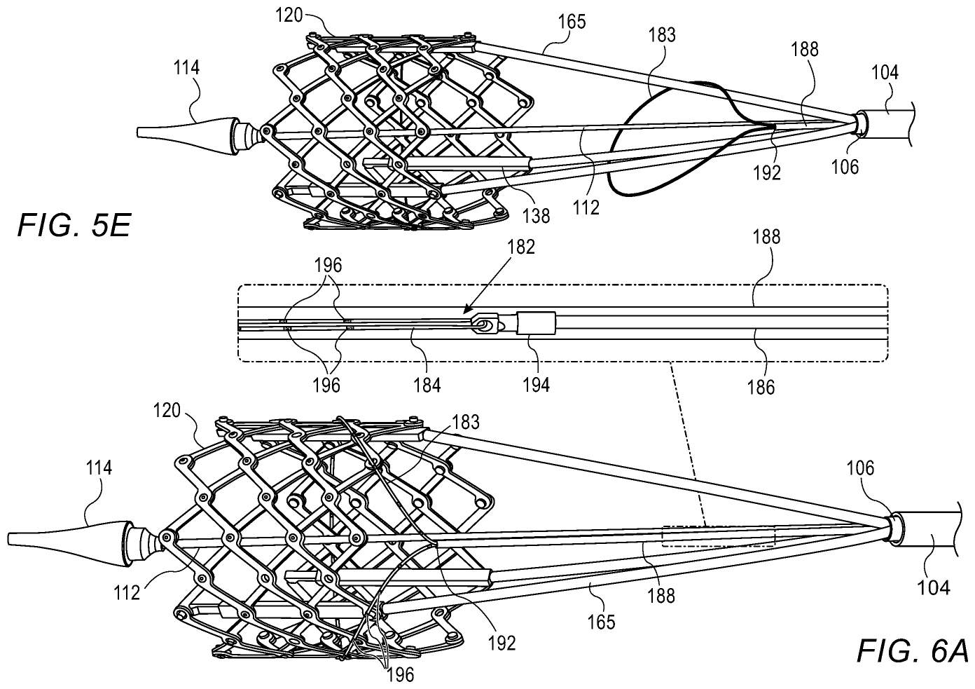

Reference is now made to FIGS. 5A-5E, showing different optional stages of utilizing a delivery assembly 100 equipped with a re-compression assembly 180. FIG. 5A shows an enlarged view of a distal portion of the delivery assembly 100, carrying a prosthetic valve 120 retained in a compressed or crimped state within a distal portion of the outer shaft 104 during delivery to the implantation site. As described above, the distal portion of the outer shaft 104 can serve as a delivery capsule that covers the crimped prosthetic valve 120. Upon reaching the desired site of implantation, the outer shaft 104 can be retracted to expose the prosthetic valve 120. FIG. 5A shows partial retraction of the outer shaft 104, exposing a distal portion of the valve 120, such as the inflow end portion 124.

FIG. 5B shows the prosthetic valve 120 exposed (i.e., no longer covered by the outer shaft 104). Certain prosthetic valves 120, such as certain mechanically expandable valves as described above in conjunction with FIGS. 2-4C, may be provided with internal resiliency promoting partial expansion thereof when extended out of a capsule or outer shaft 104. Furthermore, the mechanically expandable valve 120 may be partially expanded further to a larger diameter, prior to being locked in an irreversible manner by the engagement between the ratcheting teeth 162 and the pawl 152. For example, a proximal toothless portion of the actuator inner member 154 may be provided between the inner member's proximal end 156 and the ratcheting teeth 162, enabling axial movement between the inner and the outer members 154 and 140, respectively, along which the axial translation of the inner member 154 is reversible.

Once the valve 120 is at least partially expanded, either due to its inherent resiliency or due to active expansion thereof, prior to being in a locked state, the re-compression assembly 180 may be utilized to re-compress the valve 120 to a narrower diameter. The re-compression assembly 180 may be similarly utilized in combination with a self-expandable valve in a similar manner to the following description, once the valve is expanded, for example, if valve re-positioning is required. Similarly, as mentioned above, a re-compression assembly 180 may be utilized once a mechanically expandable valve 120 is expanded to a locked state of the actuator assemblies 138, by utilizing release members that can disengage the pawl 152 from the ratcheting teeth 162 of the actuator assemble 138.

According to some embodiments, the re-compression assembly 180 comprises a re-compression member 182 extending through the lumen of a re-compression shaft 188. The re-compression shaft 188 extends through the lumen of the delivery shaft 106. The re-compression member 182 comprises a flexible re-compression member distal segment 184, which may be formed of a flexible wire, cable, suture and the like. The flexible re-compression member distal segment 184 is configured to extend distally through an opening formed at the re-compression shaft distal end 192, and optionally surround either the valve 120 or components attached thereto, such as the support sleeves 170 of actuation arms assemblies 165.

The re-compression member 182 further comprises a re-compression member proximal segment 186, which extends through the lumen of the re-compression shaft 188 toward, and optionally into, the handle 110. In some instances, the re-compression member proximal segment 186 may be formed as a continuous extension of the flexible re-compression member distal segment 184. Alternatively, the re-compression member proximal segment 186 and the re-compression member distal segment 184 may be provided as separate components attached to each other, wherein either both segments are formed from the same materials having the same dimensions, or both are formed from the same materials but each is having different dimensions (e.g., one segment being thicker than the other), or each is formed from different materials but both are having similar dimension or dissimilar dimensions with respect to one another. For example, the re-compression member proximal segment 186 may be formed from a stiffer material than the re-compression member distal segment 184. Additionally or alternatively, the re-compression member proximal segment 186 may be formed as a thicker member than the re-compression member distal segment 184. Any of the re-compression member distal segment 184 and/or the re-compression member proximal segment 186 can be in the form of, for example, a cord, a suture, a wire, a cable or any other flexible material that can be tensioned.

A zoomed-in portion of one exemplary re-compression assembly 180 is shown in FIG. 5B. In the exemplary embodiment illustrated, the re-compression member 182 comprises re-compression member proximal segment 186 and re-compression member distal segment 184 which are separate components, attached to each other via a connector 194. According to some embodiments, two proximal ends of the re-compression member distal segment 184 are attached directly or indirectly to a distal end of the re-compression member proximal segment 186. In the exemplary embodiments shown in FIG. 5B, the re-compression member distal segment 184 is looped through a ring-like portion of a connector 194, having two parallel portions thereof extending distally from the connector 194 within the lumen of the re-compression shaft 188. The connector 194 can take any other form, configured to attach to the re-compression member distal segment 184 and to the re-compression member proximal segment 186. Alternatively, the re-compression assembly 180 may be provided without a connector 194. For example, the re-compression member distal segment 184 can be directly attached to a separate re-compression member proximal segment 186. In another example, the re-compression member distal segment 184 and the re-compression member proximal segment 186 can be integrally formed, each constituting a different region of a single continuous re-compression member 182.

As further shown in FIG. 5B, a distal portion of the re-compression member distal segment 184, extending out of the re-compression shaft distal end 192, may comprise a loop portion 183 configured to circumscribe the prosthetic valve 120. The handle 110 may be maneuvered, for example via knobs, buttons and the like, to adjust the tension on the loop portion 183. For example, a re-compression actuation mechanism (not shown) can be maneuvered at the handle 110 to either increase tension on the re-compression member 182, or release such tension, in order to readjust the diameter of loop portion 183.

Adjustment of the diameter of the loop portion 183 can be achieved, for example, by advancing the re-compression shaft distal end 192 in a distally oriented direction, relative to the re-compression shaft distal end 192, thereby reducing the loop portion diameter. Tensioning the loop portion 183 to reduce its diameter applies, in turn, an inwardly directed force on the valve 120, effective to compress the valve 120. Similarly, retraction of the re-compression shaft distal end 192 in a proximally oriented direction relative to the re-compression member distal segment 184 releases such tension, allowing the valve 120 to re-expand, either due to an internal resiliency of the frame 126, or via activation of expansion mechanisms, such as the actuator assemblies 138.

According to some embodiments, the re-compression shaft 188 is operatively connected to a re-compression actuation mechanism in the handle 110, operable by a knob, button, switch and the like. The re-compression actuation mechanism can be used to axially translate the re-compression shaft 188 in a proximal or distal direction, relative to the re-compression member 182.

It should be noted that the relative movement between the re-compression shaft 188 and the re-compression member 182 in the axial direction, refers to movement of the re-compression shaft 188 relative to the re-compression member 182, and/or movement of the re-compression member 182 relative to the re-compression shaft 188. According to some embodiments, the re-compression member distal segment 184 can be retracted in a proximally oriented direction relative to the re-compression shaft distal end 192, in order to facilitate valve compression. Similarly, the re-compression member distal segment 184 can be advanced in a distally oriented direction relative to the re-compression shaft distal end 192 in order to relieve tension and allow valve expansion.

FIG. 5B shows an exemplary state in which the valve 120 is partially expanded, after being released from the outer shaft 104. In this state, the loop portion 183 is relatively loose around the valve 120, e.g., loose enough to allow partial or full valve expansion. In some instances, the loop portion 183 may be kept in a tensioned state around a crimped valve 120 during delivery to the implantation site, thereby providing additional means by which the valve 120 is kept in a crimped diameter, which may be utilized in addition to, or instead of, covering the valve 120 within a capsule or within the distal portion of the outer shaft 104. In such cases, the diameter of the loop portion 183 may be re-adjusted during the procedure. For example, loop portion 183 may be loosened once the prosthetic valve 120 reaches a desired implantation site, and/or once the outer shaft 104 is retracted to expose the valve 120. Partial loosening of the loop portion 183 may provide control over the valve expansion diameter and rate of expansion. Further releasing the loop portion 183 may allow full expansion of the valve 120.

Thus, relative movement between the re-compression member 182 and the re-compression shaft 188 in the axial direction is effective to tighten the loop portion 183 around the prosthetic valve 120, thereby radially compressing the prosthetic valve 120. Specifically, a tensioned state of the re-compression assembly 180 is defined as a state in which the tension of the re-compression member distal segment 184 is sufficient to either compress the valve 120, or retain it such that the valve 120 cannot expand beyond a maximal diameter, dictated by the tension force of the re-compression member 182. A partially tensioned state refers to any tensioned state in which the valve 120 is partially expanded, where at any of the partially tensioned states, the valve 120 cannot expand beyond a maximal diameter (determined by the tension of loop portion 183) and wherein the maximal diameter is higher than the crimped-state diameter. A released state of the re-compression assembly 180 is defined as a state in which the tension of the re-compression member distal segment 184 does not resist valve expansion, thereby allowing free valve expansion.

While the re-compression member 182 is tightly tensed around the prosthetic valve 120 in a tensioned state of the re-compression assembly 180, it may loosely surround the prosthetic valve 120 when tension is relieved, for example in a released state, or when the diameter of the prosthetic valve 120 is lower than the maximal diameter allowable by the loop portion 183. In some cases, it may be desirable to keep the loop portion 183 tensed around the prosthetic valve 120 at all times, including in a released state, when the prosthetic valve 120 is free to expand in the radial direction. The constantly tensioned state of the loop portion 183 around the prosthetic valve 120 can be advantageous, for example, if the loop portion 183 is utilized for estimation of prosthetic valve diameter 120, as will be elaborated further below. Under such configuration, the loop portion is tightly wrapped around the outer surface of the valve 120 across the entire range of potential valve diameters, between the compressed state and the fully expanded state.

According to some embodiments, a minimal tension magnitude Ts is always applied to the re-compression member distal segment 184, and more specifically, to the loop portion 183. Such tension is configured to retain the re-compression member distal segment 184 in a minimally tensed state, even in the absence of external forces acting to collapse the valve 120. For example, the minimal tension magnitude Ts may be applied in a released state of the re-compression assembly 180. The minimal tension magnitude Ts is selected so as to apply sufficient biasing force to keep the loop portion 183 tensed around the valve 120 or other elements attached thereto, such as actuation arm assemblies 165, yet not high enough to resist valve expansion. Thus, the tension applied by the re-compression member distal segment 184 is higher than Ts in a tensed state of the re-compression assembly 180, and may be equal to Ts in a released state of the re-compression assembly 180.

FIG. 5C shows an exemplary tensioned state of the re-compression assembly 180, achieved by pulling the re-compression member distal segment 184 in a proximally oriented direction, relative to the re-compression shaft distal end 192, thereby tensioning the loop portion 183 so as to apply sufficient radial force to compress the valve 120. As shown, the position of the connector 194 in FIG. 5C is proximal relative to its position in FIG. 5B. Suh configuration enables re-positioning of the prosthetic valve 120, and/or prosthetic valve re-capturing for removal thereof from the patient.

FIG. 5D shows an exemplary released state of the re-compression assembly 180, which may be applicable, for example, upon reaching a desired implantation site (e.g., after valve repositioning within the patient's body). In this state, the re-compression member distal segment 184 is released, allowing the valve 120 to re-expand, for example to a fully expanded diameter. As shown, the position of the connector 194 in FIG. 5D is distal relative to its position in FIG. 5C or in FIG. 5B.

According to some embodiments, the re-compression shaft 188 and the re-compression member proximal segment 186 may be retracted, as shown in FIG. 5E, for example by maneuvering the handle 110 to pull them in a proximally oriented direction, wherein the loop portion 183 no longer tightly surrounds the valve 120. Retraction of the re-compression assembly 180 may be performed during the implantation process, for example, to allow unhindered expansion of the valve 120. Alternatively or additionally, the re-compression assembly 180 may be retracted in lieu of delivery apparatus 102 retraction, for example, after completing the valve positioning and expansion procedures.

Prosthetic valve expansion against the surrounding tissue may pose a variety of risks associated with a mismatch between the valve expansion diameter and the surrounding tissue. One complication is related to valve over-expansion, which may exert excessive radial forces on the surrounding anatomy, resulting in potential damage to the tissue or even annular rupture. On the other hand, valve under-expansion might increase the risk of aortic valve or mitral valve regurgitation. Inappropriate expansion may also result in unfavorable hemodynamic performance across the valve 120, such as increased pressure gradients or flow disturbances resulting from diameter mismatch, which may be associated with increased risk of thrombus formations.

Thus, in order to avoid the deleterious effects of either annular rupture, inferior hemodynamic performance or valve regurgitation, arising due to either over-expansion or under-expansion, respectively, of the valve frame 126, a clinician should be able to control the degree of frame 126 expansion according to real-time feedback received during the procedure, indicating, for example, current valve diameter and/or expansion force.

According to an aspect of the invention, the re-compression assembly 180 is configured to provide real-time feedback, such as visual or auditory real-time feedback, regarding the radial expansion diameter of the prosthetic valve 120.

According to some embodiments, the re-compression member distal segment 184 comprises at least one radiopaque marker 196. The at least one, and optionally a plurality of, radiopaque markers 196, may span along at least a portion of, and preferably along the entire length of, the loop portion 183. According to some embodiments, the at least one, and optionally a plurality of, radiopaque markers 196, span along the entire length of the re-compression member distal segment 184. A radiopaque marker 196 comprise a radiopaque material, understood to be capable of producing a relatively bright image on a fluoroscopy screen or another imaging technique, during the prosthetic valve 120 implantation procedure. Radiopaque materials can include, but are not limited to, gold, platinum, tantalum, tungsten alloy, platinum iridium alloy, palladium, and the like.

As noted, the loop portion 183 may be configured to be tightly wrapped around the external surface of the valve 120 at all times, either during a tensioned state or during a released state of the re-compression assembly 180, due to the minimal tension magnitude Ts applied on the re-compression member distal segment 184. Thus, the at least one, and optionally a plurality of, radiopaque markers 196 disposed along the loop portion 183 wrapped around the valve 120, may provide a real-time visually detectable indication of the diameter of the prosthetic valve 120.

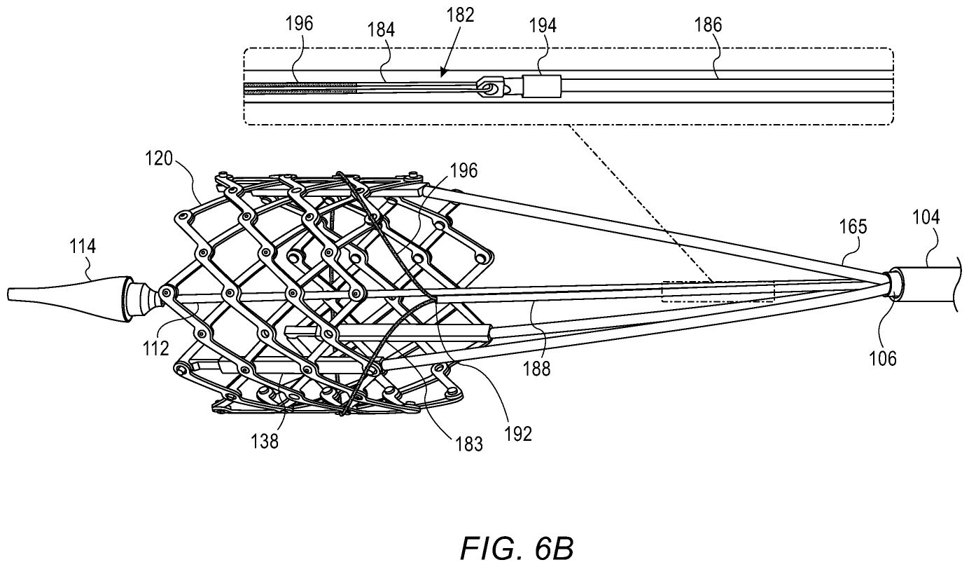

FIGS. 6A-6B show different configurations of radiopaque markers 196 disposed along at least a portion of the re-compression member distal segment 184, according to some embodiments. FIG. 6A shows a variant of the radiopaque markers 196, provided in the form of a plurality of radiopaque bands disposed along at least a portion of the length of the re-compression member distal segment 184. According to some embodiments, a plurality of radiopaque markers 196, such as radiopaque marker bands, may be spaced at known distances from each other, for example along a portion the loop portion 183, such that the radiopaque marker bands 196 could be used to provide visual estimates of the diameter of the prosthetic valve 120. The plurality of radiopaque markers 196 may be spaced from each other along at least a portion of the loop portion 183 at any desired pattern. For example, the plurality of radiopaque markers 196 may be equally spaced from each other, or may be spaced at varying distances from each other.

According to some embodiments, the plurality of radiopaque markers 196 are disposed along the loop portion 183 at various positions, thereby providing visual indication of the valve diameter. For example, the plurality of radiopaque markers 196 may span along a portion of the loop portion 183, which is substantially equal to at least half of the prosthetic valve perimeter when expanded to a maximal diameter, so as to ensure detection of any sub-maximal diameters. Similarly, the positions of the plurality of radiopaque markers 196 may be set to cover at least half of the prosthetic valve perimeter when fully expanded, and circumscribed by the loop portion 183. In some instances, it may be preferable to cover the entire loop portion 183 with the plurality of radiopaque markers 196 in order to compensate for situations in which the marked region is not aligned with the angle of view during fluoroscopy.

According to some embodiments, radiopaque markers 196 can be formed by means of radiopaque inks and adhesives, and applied on at least a portion of the re-compression member distal segment 184 in a number of ways, such as screen printing, high speed roller printing, coating, dipping, etc. According to yet further embodiments, the markers can provided as separately formed components, such as annular rings or C-shaped bands that are mounted on the re-compression member distal segment 184.

According to some embodiments, as shown in FIG. 6B, a single radiopaque marker 196 is disposed along a minimal marking length, at a position which preferably corresponds to the contact region between the loop portion 183 and the perimeter of the prosthetic valve 120. A minimal marking length may be chosen so as to enable diameter estimation of the valve 120, across its entire range of diameters. For example, the minimal marking length can correspond to the perimeter of the valve 120, in a range between the minimal crimping diameter and the maximal expansion diameter. According to some embodiments, the minimal marking length is at least as great as the perimeter of the prosthetic valve 120 when fully expanded. According to some embodiments, the entire length of the re-compression member distal segment 184 comprises a single continuous radiopaque marking 196.

According to some embodiments, the radiopaque marking is formed as a radiopaque coating 196, such that the re-compression member distal segment 184 is coated by a radiopaque material along a minimal marking length, which optionally can include its entire length.

According to some embodiments, the connector 194 is a releasable connector, configured to releasably attach the re-compression member proximal segment 186 to the re-compression member distal segment 184. FIGS. 7A-7C show an exemplary embodiment of a delivery assembly 100 equipped with a re-compression assembly 180 having a releasable connector 194, according to some embodiments. FIG. 7A shows the re-compression assembly 180 in a state wherein re-compression member proximal segment 186 is connected to the re-compression member distal segment 184 via the releasable connector 194. According to some embodiments, the releasable connector 194 comprises a proximal connector element 193 and a distal connector element 195, releasably attached to each other. The re-compression member proximal segment 186 is coupled to the proximal connector element 193, while the re-compression member distal segment 184 is coupled to the distal connector element 195. In the illustrated example, a re-compression member distal segment 184 may loop through an eyelet formed in the distal connector element 195, though any other type of coupling is contemplated.

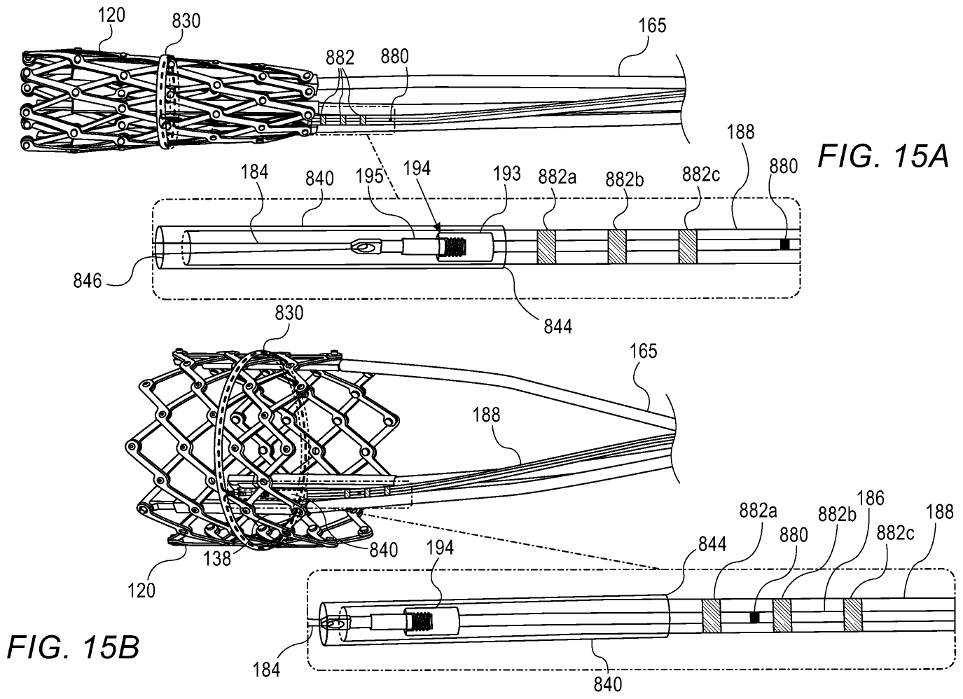

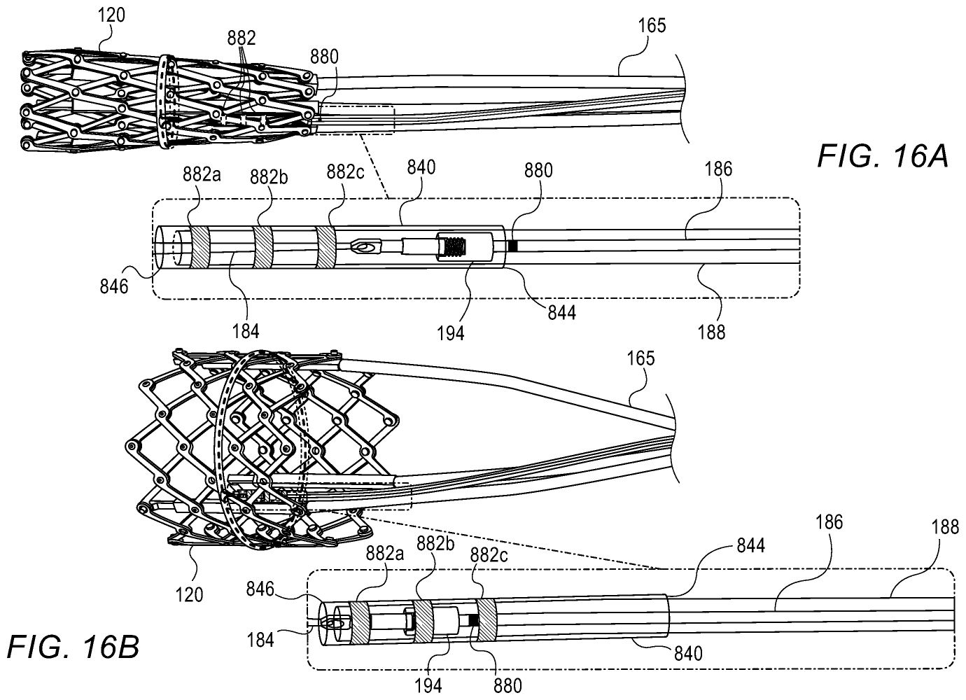

In some applications, the loop portion 183 of the re-compression member distal segment 184 may extend through a circumferential sleeve circumscribing the valve 120. In the exemplary embodiment illustrated in FIGS. 7A-7C, an outer skirt 137 comprises a sleeve 132 integrally formed therewith, for example along a proximal edge of the outer skirt 137. The sleeve 132 can be provided with an opening 133 through which the re-compression member distal segment 184 may extend into a lumen of the sleeve 132. While the sleeve 132 is shown in FIGS. 7A-7C as integrally formed with, or attached to (e.g., sewn to), the outer skirt 137, it will be clear that in alternative applications, a stand-alone sleeve can be provided around the valve 120, such as the circumferential sleeve 830 illustrated (as shown, for example, in FIGS. 15A-16B). Moreover, while shown in FIGS. 7A-7C in conjunction with a re-compression assembly 180 having a releasable connector 194, it will be clear that the re-compression member distal segment 184 looped around the valve 120 according to any other embodiments of the invention, such as the embodiments described and illustrated in conjunction with FIGS. 6A-6B, may similarly extend through sleeves 130 or 830.

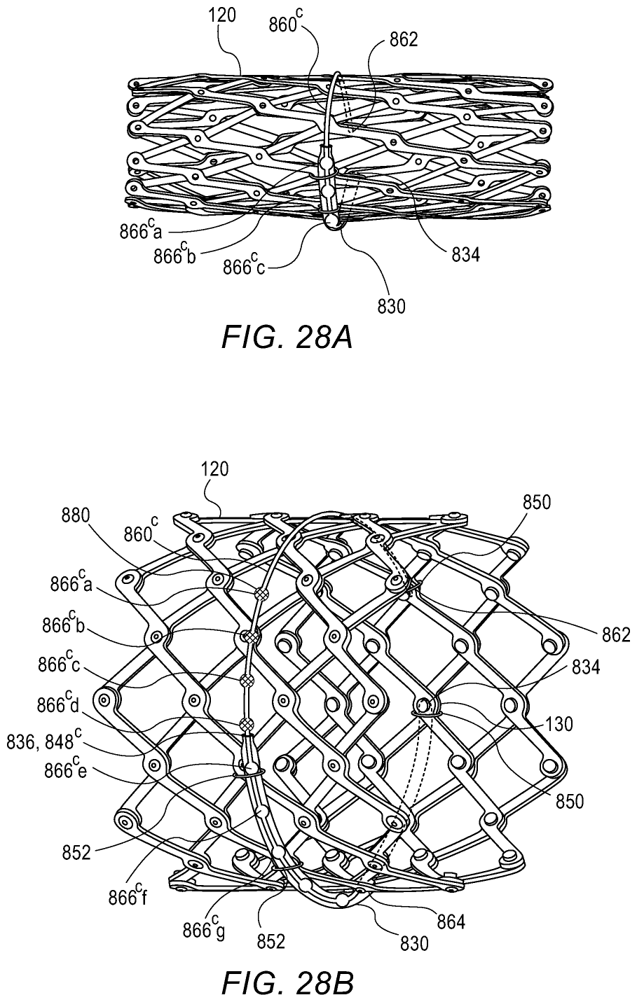

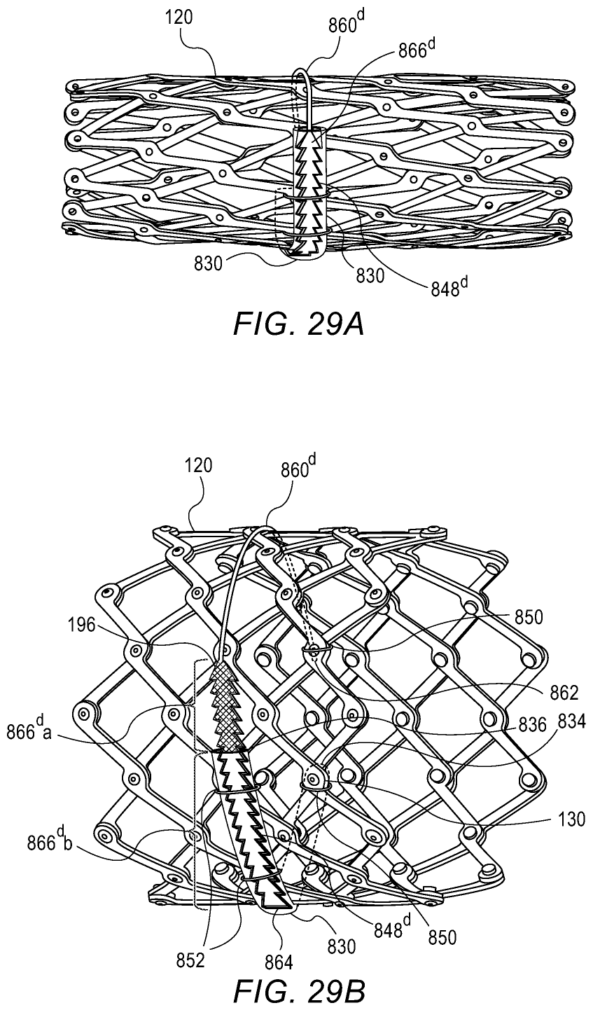

The sleeve 132, 830 circumscribing the valve 120 is configured to retain at least a portion of the re-compression member distal segment 184 around at least a portion of the circumference of the valve 120. In some applications, the sleeve may be disposed around the entire circumference, such as shown for sleeve 132 in FIGS. 7A-7C and for sleeve 830 in FIGS. 15A-16B. In some applications, the sleeve cab be disposed around a portion of the circumference of the valve 120, such as shown for sleeve 830 in FIGS. 28A-29B. In some applications, a circumferential sleeve circumscribing the valve 120 may comprise a plurality of sleeve-portions (not shown), disposed around a circumference of prosthetic valve 120, circumferentially spaced from each other.

According to some embodiments, the valve 120 further comprises a guide member 840 disposed between the outflow end 123 and the sleeve 132, 830. The guide member 840 is provided with a guide member lumen 842, defined between a guide member proximal end 844 and a guide member distal end 846. The guide member proximal end 844 can be positioned in alignment with, or distal to, the outflow end 123. The guide member distal end 846 is positioned proximal to the guide member sleeve 132, 830, and more specifically, can be positioned proximal to the guide member sleeve opening 133, 833.

At least a portion of the re-compression member 182 extends through the guide member lumen 842, and is axially movable there-through. In the illustrated example, a proximal portion of the re-compression member distal segment 184, the releasable connector 194, and a distal portion of the re-compression member proximal segment 186, may extend through and be axially movable within the member 840.

The re-compression member distal segment 184 can include a plurality of radiopaque markers 196 as described and illustrated in conjunction with FIG. 6A, or a single radiopaque marker 196 disposed along a minimal marking length thereof, as described and illustrated in conjunction with FIG. 6B. The sleeve 132, 830 can comprise a radiolucent material or include a cut-out window, so as to allow visibility of radiopaque markers 196 under fluoroscopy, thereby enabling the re-compression assembly 180, shown in FIGS. 7A-7B, to be utilized for providing real-time detectable indications of the diameter of the prosthetic valve 120 according to any of the embodiments described and illustrated in conjunction with FIGS. 6A-6B.

Once the desired diameter of the prosthetic valve 120 is reached, at least a portion of the re-compression assembly 180 may be released from the valve 120. Specifically, as shown in FIG. 7B, the re-compression member proximal segment 186 can be released from the re-compression member distal segment 184, which may in turn remain around the expanded valve 120.

According to some embodiments, the distal connector element 195 may be provided with external threads, which may be engaged with a threaded bore of the proximal connector element 193. It will be clear that in alternative applications, the distal connector element 195 may be provided with a threaded bore, and the proximal connector element 193 may be provided with matching external threads. In embodiments wherein the distal connector element 195 and the proximal connector element 193 are threadedly engaged with each other, the re-compression member proximal segment 186 may comprises a relatively rigid material, formed as a torque transferring wire, cable and the like.

As shown in FIG. 7B, the proximal connector element 193 can be released from the distal connector element 195, and proximally pulled along with the re-compression member proximal segment 186, for example through the lumen of the re-compression shaft 188. FIG. 7C shows a further step of pulling the re-compression shaft 188 from the guide member 840. In some applications, a distal portion of the re-compression shaft 188 is disposed within the guide member lumen 842. Additionally or alternatively, a distal portion or a distal end of the re-compression shaft 188, is releasably attached to the guide member 840.

The guide member 840 may be formed as a rigid hollow member, such as a tube or any other hollow member with other circular or non-circular cross-sections. The guide member 840 is rigidly attached to the frame 126, directly or indirectly (e.g., via another component of the prosthetic valve 120 attached to the frame 126). According to some embodiments, the guide member 840 may be attached a commissure post or a component of the actuator assembly 138, such as the actuator outer member 140 as shown in FIGS. 7A-7C, wherein attachment may be accomplished by welding, gluing, soldering and the like. Alternatively, the guide member 840 can be attached to the frame 126, such as to at least one junction 130 (alternative embodiments not shown).

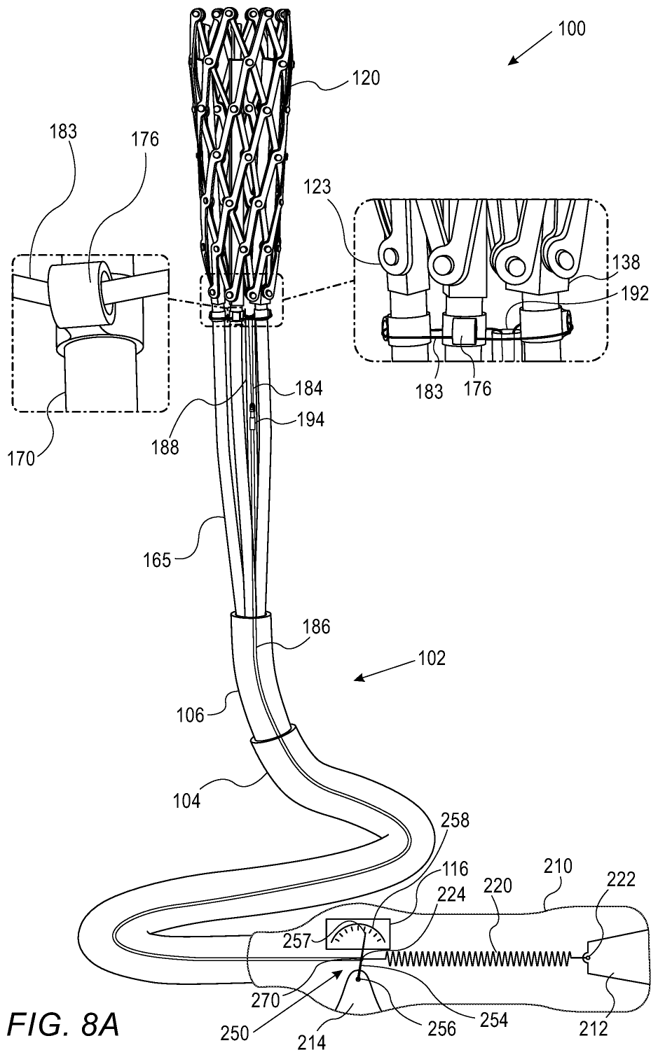

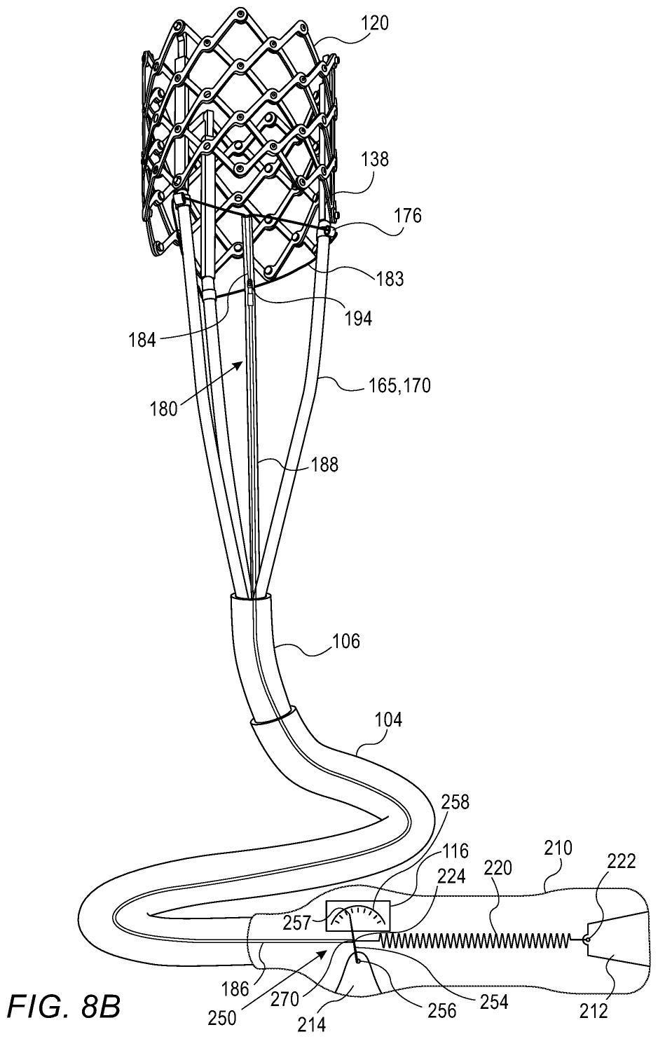

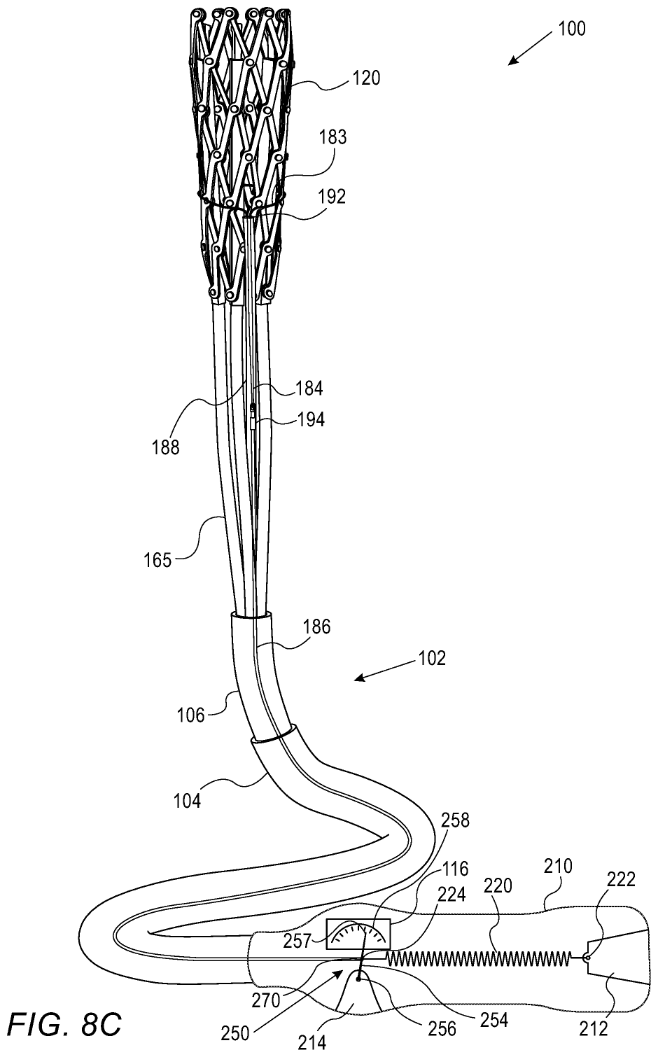

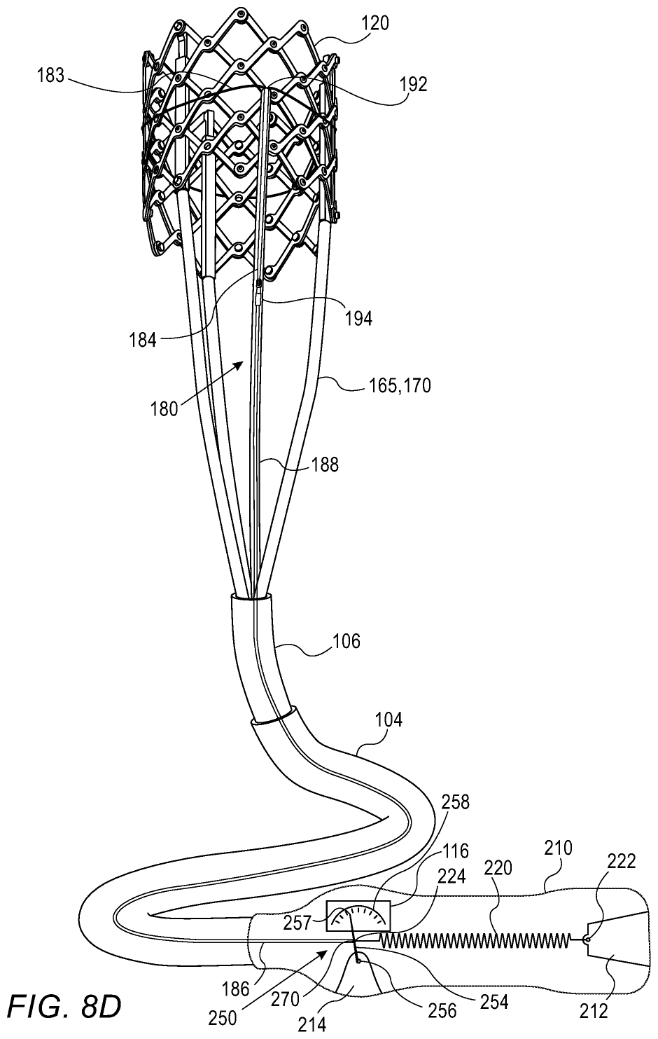

Another example of a re-compression assembly 180 is shown in FIGS. 8A-8B, wherein the re-compression member distal segment 184 comprises a distal loop portion 183 which is wrapped around, or extending between, the support sleeves 170, instead of circumscribing the external surface of the prosthetic valve 120. As shown in FIG. 8A, each support sleeve 170 can include a loop attachment member 176 in the vicinity of its distal end 172. The re-compression member distal segment 184, and more specifically, the loop portion 183, is connected to, and extending between, the loop attachment members 176 of the actuator arm assemblies 165. For example, the loop portion 183 may be threaded through eyelet-shaped loop attachment members 176, as shown in a zoomed-in view of the attachment region between the actuator arm assemblies 165 and the prosthetic valve 120 on the top-right of FIG. 8A, and a zoomed-in region of the distal portion of a single support sleeve 170 having a loop attachment member 176 on the top-left of FIG. 8A.

The loop attachment members 176 can be in the form of eyelets, hooks, rings, clips, apertures within the support sleeves 170 and/or the actuation members 166, and any other structural elements configured to retain there-between, and enable extension of, the re-compression member distal segment 184, and more specifically, the loop portion 183.

According to some embodiments, relative movement between the re-compression member 182 and the re-compression shaft 188 in the axial direction, is effective to apply tension to the loop portion 183 connected to, resulting in radial inward movement of the actuation arm assemblies 165, thereby radially compressing the prosthetic valve 120. FIG. 8A illustrates the loop portion 183 extending between the support sleeves 170 in a tensioned state of the re-compression assembly 180, applying inwardly directed force on the actuation arm assemblies 165. As long as the actuation members 166 are attached to the actuator assemblies 138, the frame 126 of the valve 120 is also proportionally radially compressed.

Tensioning of the re-compression assembly 180 shown in FIG. 8A may be achieved by maneuvering the handle so as to pull the re-compression member proximal segment 186 in a proximally oriented direction relative to the re-compression shaft 188. Tension release may be achieved by releasing the pulling force, allowing the re-compression member proximal segment 186 to translate in a distally oriented direction as the prosthetic valve 120 expands.

FIG. 8B illustrates a released state of the re-compression assembly 180, wherein the prosthetic valve 120 is allowed to expand relative to its compressed state in FIG. 8A. As illustrated, the loop portion 183 is tightly extended between the actuation arm assemblies 165 both in the expanded state (see FIG. 8B) and the compressed state (see FIG. 8A) of the prosthetic valve 120, for example due to the minimal tension magnitude Ts applied to the re-compression member 182.

According to some embodiments, the re-compression shaft 188 is immovable in an axial direction, such that adjustment of the tension applied to the loop portion 183 may be facilitated by either applying, or releasing a pulling force, on the re-compression member proximal segment 186. Thus, axial translation of the re-compression member proximal segment 186 is proportional to the perimeter of the loop portion 183, which in turn is proportional to the diameter of the prosthetic valve 120, as long as the actuation arm assemblies 165, to which the loop portion 183 is connected, are coupled to the prosthetic valve 120 (e.g., to the valve actuator assemblies 138).

According to some embodiments, the delivery apparatus 102, and more specifically the handle, further comprises a diameter gauge. The diameter gauge is coupled to the re-compression assembly at a gauge coupling point, such that expansion or contraction of the prosthetic valve 120, when attached to the actuation arm assemblies 165, is effective to axially translate the position of the gauge coupling point. The diameter gauge is configured to provide real-time indication of the valve diameter, based on axial position and/or axial translation of the gauge coupling point.

According to some embodiments, the real-time indication provided by the diameter gauge is a visual real-time indication. According to some embodiments, the real-time indication provided by the diameter gauge is a signal (e.g., an electric signal or an optic signal) generated by the diameter gauge. According to some embodiments, the diameter gauge is coupled to the re-compression member proximal segment 186 at a gauge coupling point.

FIGS. 8A-8B show an exemplary embodiment of a handle 210, which may be substantially similar to handle 110. The main difference is that handle 210 further comprises a diameter gauge 250 coupled to the re-compression member proximal segment 186, and configured to provide real-time indication of the diameter of the prosthetic valve 120, based on axial position and/or axial translation of the gauge coupling point 270, as will be elaborated in further detail below.

According to some embodiments, the re-compression member proximal segment 186 extends into the handle 210, for example in order to connect with an internal mechanism housed within the handle 210, configured to maneuver the re-compression assembly 180 between a released state and a tensioned state thereof, including a variety of partially tensioned states that may correspond to a variety of respective valve maximal diameters.

According to some embodiments, the handle 210 may include a user operable element, such as steerable or rotatable adjustment knobs, levers, sliders, buttons (not shown) and the like, configured to allow a user to adjust the tensioning force applied to the re-compression member 182. Additionally or alternatively, the handle 210 may comprise an automated mechanism configured to re-adjust such tensioning force according to input received from a user interface or from sensors operably coupled to components of the delivery apparatus 100.

In some cases, tension applied to the re-compression member 182, for example by applying a certain magnitude of a pull-force thereto, may extend length of the re-compression member 182 to a certain degree, relative to its length in a released state, or relative to its length at other pull force magnitudes that may be applied thereto. Such changes in the length of the re-compression member 182 may alter the position of the re-compression member proximal segment 186 within the handle. Lengthening of the re-compression member 182 may result in inaccuracies in valve diameter estimation, as indicated by a diameter gauge, which is based on the position of the re-compression member proximal segment 186, relative to the diameter gauge.

According to some embodiments, the re-compression member proximal segment 186 is connected within the handle 210 to a spring 220. The spring 220 may be affixed to a spring support member 212 of the handle 210 at a spring first end 222. A spring second end 224, opposite to the spring first end 222, may be connected to the re-compression member proximal segment 186.

The spring 220 is configured to apply an axially oriented pull-force on the re-compression member proximal segment 186 in a released state. The magnitude of the force applied by the spring 220 is sufficient to apply the minimal tension magnitude Ts to the loop portion 183, in the absence of other external forces effective to tighten the loop portion 183 around the actuation arm assemblies 165.

FIGS. 8A-8B schematically illustrate the interior of the handle 210, wherein the re-compression member proximal segment 186 is shown to extend all the way into the handle 210 through the lumen of the delivery shaft 106. The spring 220 is schematically illustrated, having the first spring end 222 affixed to spring support member 212 of the handle 210, at a position proximal to the attachment point between the spring second end 224 and the re-compression member proximal segment 186. In such a configuration, the spring 220 is preferably a coil compression spring, having a spring coefficient suitable to pull the re-compression member proximal segment 186 by a pull force that matches the desired minimal tension magnitude Ts. Advantageously, applying the minimal tension magnitude Ts to the loop portion 183 allows more accurate measurement of prosthetic valve diameter.

It will be understood that the spring first end 222 and the spring second end 224 may be positioned in any number of alternative locations within the handle 210, which may dictate the type of spring utilized in conjunction with the re-compression assembly 180. For example, if the spring first end 222 is positioned distal to the spring second end 224 (configuration not shown), it may be necessary to implement an extension spring instead of a compression spring, such that the spring 220 may extend the re-compression member proximal segment 186 attached to its second end 224 in a proximally oriented direction, to apply the minimal tension magnitude Ts on the loop portion 183, as described above.

According to some embodiments, the spring first end 222 comprises a hook, an eyelet, a ring and the like, by which it may be coupled to the spring support member 212 of the handle 210. According to some embodiments, the spring's second end 224 comprises a hook, an eyelet, a ring and the like, by which it may be coupled to the re-compression member proximal segment 186, a dial 254 (described below), or both.

According to some embodiments, the handle 210 includes a user-operable mechanism (not shown in FIGS. 8A-8B) connected to the re-compression member proximal segment 186 and configured to pull the re-compression member proximal segment 186 in a proximally oriented direction, to compress the prosthetic valve 120 and/or retain it in a maximal desired diameter. Since the tension magnitude applied to the loop portion 183 in a tensioned state of the re-compression assembly 180, is higher than the minimal tension magnitude Ts, the pulling force applied to the re-compression member proximal segment 186 in a tensioned state, is higher than the pulling force applied by the spring 220 in a released state.

When tension is relieved in a released state of the re-compression assembly 180, the only pull force applied to the re-compression member proximal segment 186 is the pull force of the spring 220. This, in turn, allows the re-compression member proximal segment 186 to translate in a distally oriented direction if the prosthetic valve 120 is expanded, thereby extending the spring 220 (in the case of a compression spring) in the same direction, as shown in FIG. 8B.

Various types of springs can be used instead of a coil compression spring 220, such as tension springs, torsion springs or leaf springs. Alternatively, the spring 220 can be replaced and/or additionally accompanied by other biasing members, such as stretchable and/or elastic cords, elastomeric bodies (e.g., a silicone of polyurethane component) which is compressible under external force application, and returns to its original shape when such force is removed. Any such biasing member may replace a coil compression spring 220 as long as it applies a biasing force sufficient to apply a minimal tension magnitude to the loop portion 183.

According to some embodiments, the handle 210 comprises a diameter gauge 250 coupled to the re-compression member proximal section 186 at a gauge coupling point 270. The diameter gauge 250 is configured to provide a real-time visual indication of the diameter of the prosthetic valve 120, based on axial position and/or axial translation of the gauge coupling point 270 within the handle 210. The state of the diameter gauge 250 may be visible through a visual interface, such as the display 116. In other words, the diameter gauge 250 may be configured to provide real-time visual indication of the diameter of the prosthetic valve 120 via the display 116.

According to some embodiments, as shown in FIGS. 8A-8B, the diameter gauge 250 comprises a dial 254 coupled, directly or indirectly, to the re-compression member proximal segment 186, at a gauge coupling point 270. The dial may be pivotably attached to a dial support member 214 of the handle 210 via a dial pivot 256. The dial 254 is configured to rotate angularly about the dial pivot 256 when the re-compression member proximal segment 186 translates in an axial direction.

According to some embodiments, the diameter gauge 250 comprises a scale or indicator marks 258, wherein each indicator mark may be in the form of a numerical value, or any other symbol, representative of a specific diameter. The range of the indicator marks 258 may be chosen to reflect the range of prosthetic valve diameters between the compressed state and the expanded state. A dial tip 257, which may be the free end of the dial 254, opposite the dial pivot 256, points toward the indicator marks 258. The dial tip 257 is configured to point at the indicator mark 258 representing of the current diameter of the prosthetic valve 120.

According to some embodiments, the display 116 comprises a window, through which the indicator marks 258 and the dial tip 257 are visible to an outside viewer (e.g., an operator of the delivery assembly 100). According to some embodiments, the indicator marks 258 comprise color marks, such as green, yellow, red and so on, to provide visual indication of safe or dangerous zones, for example. According to some embodiments, the display 116 includes the indicator marks 258.

The diameter gauge 250 is configured to translate the axial movement, and/or axial position, of the gauge coupling point 270, which move along with the re-compression member proximal segment 186, to a corresponding position of the dial 254, pointing at an indicator mark representative of the current valve diameter, based on predetermined mathematical relationship. For example, application of an axial pull force on the re-compression member proximal segment 186, which changes its position within the handle 210, is translated to tensioning applied to the loop portion 183. Such tensioning applies radially inward force on the actuation arm assemblies 165, resulting in a proportional change in the perimeter of the loop portion 183 wrapped around, and/or extended between, the actuation arm assemblies 165, thereby forcing the actuation arm assemblies 165 to move radially inward, toward each other.

In the examples of FIGS. 8A-8B, the loop portion 183 can be approximated as a substantially triangularly shaped loop. Since the prosthetic valve 120 is coupled to the actuation arm assemblies 165, the valve diameter proportionally changes in response to the inwardly directed movement of the actuation arm assemblies 165. Assuming that the loop attachment members 176 are located in the vicinity of the support sleeve distal ends 172, the loop portion 183 is in close proximity to the valve outflow end 123. In such cases, the perimeter of the valve outflow end 123 may be assumed to constitute, in close approximation, a circular perimeter encircling the triangularly-shaped loop portion 183. This relationship can be used to derive the diameter of the valve 120.

It will be clear that the above mentioned relationship is described in a simplified manner, only to demonstrate the conceptual principles by which the valve diameter can be derived from an axial translation or axial position of the re-compression member proximal segment 186. Such relationships may be further adjusted to improve the accuracy of the measurement. For example, the actual shape of the loop portion 183 may be more complex, due to the influence of the position of the re-compression shaft distal end 192 relative to the loop attachment members 176. Moreover, the number of actuation arm assemblies 165 may be other than three, resulting in other, potentially more complex, loop portion contours.

As indicated above, once the re-compression member proximal segment 186 is released, the prosthetic valve 120 is free to expand, either due to the internal resiliency of the frame 126, or due to active expansion of the prosthetic valve 120, for example by utilizing the mechanical expansion mechanism described above. During valve expansion, the actuation arm assemblies 165 expand radially outward, thereby enlarging the perimeter of the loop portion 183, which in turn axially translates the re-compression member proximal segment 186, along with the gauge coupling point 270, in a distally oriented direction.

As shown in FIG. 8B, valve expansion, accompanied by axial translation of the re-compression member proximal segment 186, along with the gauge coupling point 270, in a distally oriented direction, acts to rotate the dial 254 in the appropriate direction, for example, counterclockwise as shown in FIG. 8B, relative to FIG. 8A. As a result, the position of the dial tip 257 changes, pointing at the indicator mark 258, representing the valve diameter or a close approximation thereof.

While the gauge coupling point 270 is shown in the exemplary embodiment of FIGS. 8A-8B as an attachment point between the dial 254 and the proximal end of the re-compression member proximal segment 186, it will be understood that this is a simplified, non-binding, schematic representation of the gauge coupling point 270 position, and that any other portion of the re-compression member proximal segment 186 may be coupled, directly or indirectly, to the dial 254.

While the re-compression member proximal segment 186 is shown in the exemplary embodiment of FIGS. 8A-8B attached at its proximal end to the spring second end 224, it will be understood that this is a simplified, non-binding, schematic representation of the coupling between the re-compression member proximal segment 186 and the spring 220, and that any other portion of the re-compression member proximal segment 186 may be coupled, directly or indirectly, to any other portion of the spring 220.

FIGS. 8C and 8D show a re-compression assembly 180 having a re-compression member proximal segment 186 coupled to a diameter gauge 250, in a compressed state and an expanded state of the prosthetic valve 120, similar to the views shown in FIGS. 8A-8B, except that the re-compression assembly 180 is provided with a loop portion 183 configured to circumscribe the prosthetic valve 120 in the same manner illustrated and described in conjunctions with FIGS. 5A-5E. All other embodiments described in FIGS. 8A-8B are similarly applicable to the re-compression assembly 180 shown in FIGS. 8C-8D.

In the examples of FIGS. 8C-8D, the loop portion 183 can be approximated as a substantially circular loop, having its perimeter changing along with the perimeter of the prosthetic valve encircled thereby. This configuration may represent a simple relationship between the perimeter of the loop portion 183 and the valve diameter, which can be utilized to derive the diameter of the valve 120.

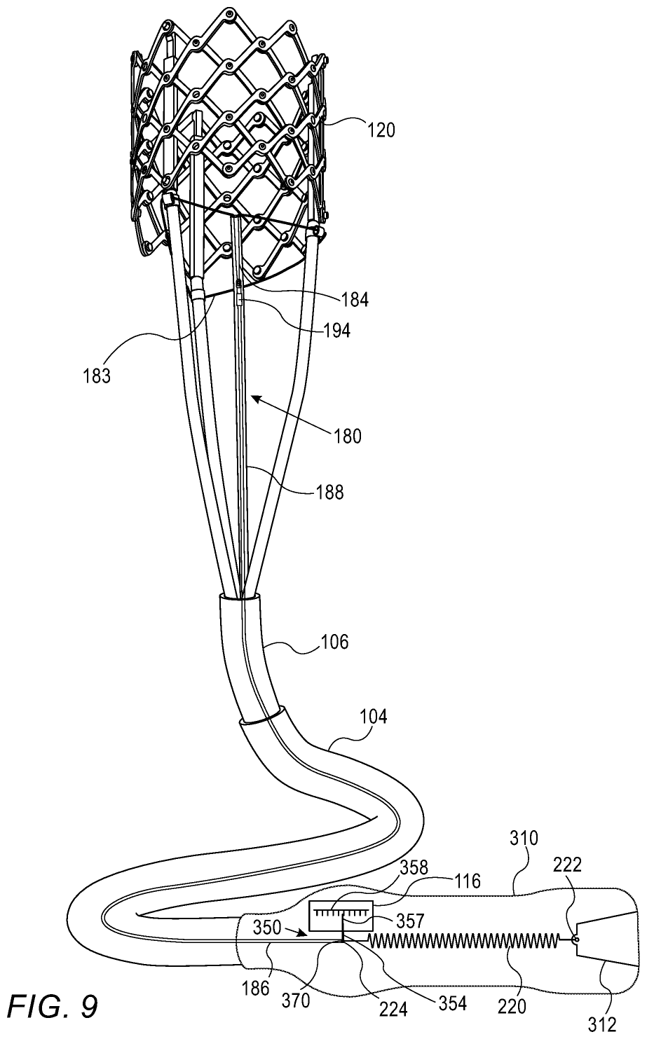

FIG. 9 shows another configuration of handle 310 equipped with a diameter gauge 350, comprising a dial 354 attached to the re-compression proximal segment 186 at a gauge coupling point 370. The dial 354 comprises a distal tip 356 pointing at a scale or indicator marks 358. The handle 310 is similar to the handle 210, except that it does not necessarily include a dial support member. The diameter gauge 350 is similar to diameter gauge 250, except that the dial 354 is not pivotable around a pivot, and is not connected to a dial support member. The non-pivotable dial 354 may be oriented substantially orthogonal to a longitudinal axis of the re-compression proximal segment 186. Thus, the dial 354 moves along with the re-compression proximal segment 186 when it translates in an axial direction, having the dial tip 357 pointing at the indicator marks 358 so as to indicate the current diameter of the prosthetic valve 120, based on the principles described and illustrated in conjunction with FIGS. 8A-8B.

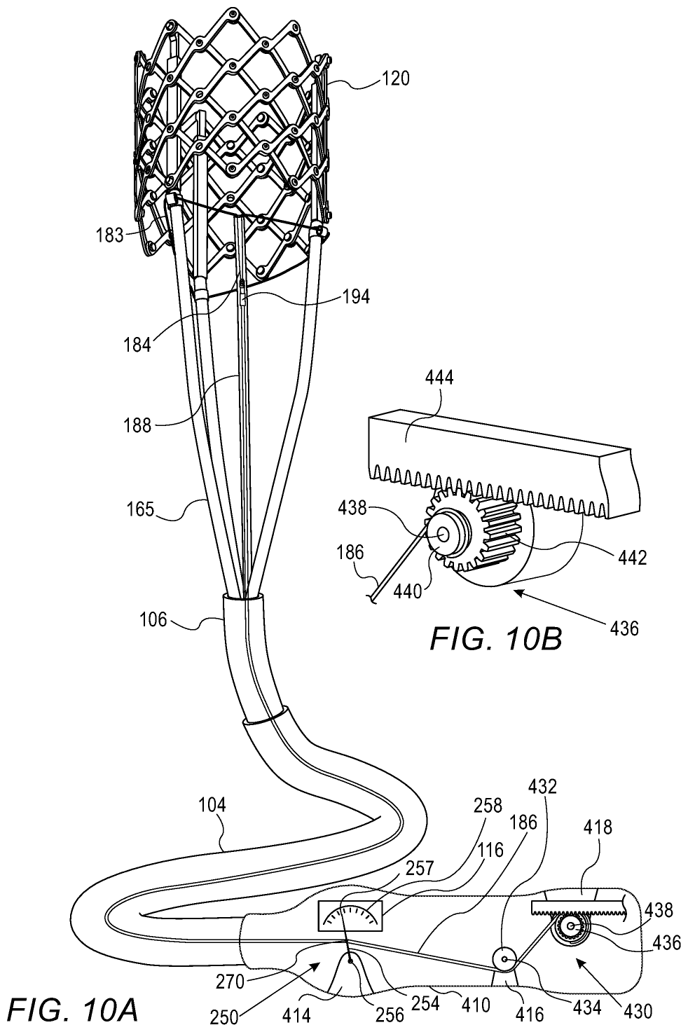

An additional embodiment of a handle 410 comprising the diameter gauge 250, and maneuverable to control the re-compression assembly 180, is shown in FIG. 10A. Handle 410 may be substantially similar to handle 210, except that it comprises a pulley assembly 430 having first and second pulleys 432 and 436, respectively. The first pulley 432 is mounted to any portion of the handle 410. According to some embodiments, the first pulley 432 is connected via a first pin 434 to a first pulley support member 416 of the handle 410. The second pulley 436 can be mounted to any portion of the handle 410, and may be laterally and/or axially offset from the first pulley 432. According to some embodiments, the second pulley 436 is connected via a second pin 438 to a second pulley support member 418 of the handle 410. The first and second pulleys 432 and 436 are freely rotatable about the first and second pins 234 and 238, respectively.

In the embodiment illustrated in FIG. 10A, the re-compression member proximal segment 186 is routed through the pulley assembly 430 within the handle 410. For example, the re-compression member proximal segment 186 can be routed partially around the first pulley 432 and around the second pulley 436. According to some embodiments, the re-compression member proximal segment 186 can be connected to, and configured to wrap around, the second pulley 436. The pulley assembly 430 may be adjusted apply a minimal tension magnitude Ts to the re-compression member 182 at all times, including in a released state, without the need for a spring 220 attached to the re-compression member proximal segment 186. According to other embodiments, a spring 220 is attached to the re-compression member proximal segment 186 according to any of the embodiments described above, in addition to the re-compression member proximal segment 186 being routed through the pulley assembly 430.

According to some embodiments, the pulley assembly 430 may include one or more additional pulleys around which the re-compression member proximal segment 186 may be routed. According to some embodiments, the second pulley 436 comprises a pole portion 440, around which the re-compression member proximal segment 186 may wrap around, and a gear portion 442. The gear portion 442 may be configured to be engaged, for example, with a corresponding rack 444. FIG. 10B constitutes a zoomed-in view in perspective of the second pulley 436 engaged with a rack 444. The rack 444 may be attached, directly or indirectly, to a user controllable element, such as steerable or rotatable adjustment knobs, levers, sliders, buttons (not shown) and the like, configured to allow the user to control the re-compression assembly 180 by adjusting the tensioning force applied to the re-compression member 182.

The user controllable element may be maneuvered to axially translate the rack 444 in one direction, thereby rotating the second pulley 436 in a corresponding direction, for example to promote further wrapping around of the re-compression member proximal segment 186 around the pole portion 440. Similarly, the user controllable element may be maneuvered to axially translate the rack 444 in an opposite direction, thereby unwrapping the re-compression member proximal segment 186 from the pole portion 440, relieving tension from the loop portion 183.

While FIGS. 10A-10B illustrate a drive mechanism including a rack 444 and a gear 442, it will be understood that any other drive mechanism, utilized to control the direction of rotation of the second pulley 436, is contemplated.

According to some embodiments, the handle 410, as illustrated in FIGS. 10A-10B, comprises the diameter gauge 350 instead of the diameter gauge 250, wherein the re-compression member proximal segment 186 is coupled to the dial 354 at the gauge coupling point 370. In such embodiments, the dial 354 moves along with the re-compression proximal segment 186 when it translates in an axial direction, having the dial tip 357 pointing at the indicator marks 358 so as to indicate the current valve diameter, as described above in conjunction with FIG. 9.

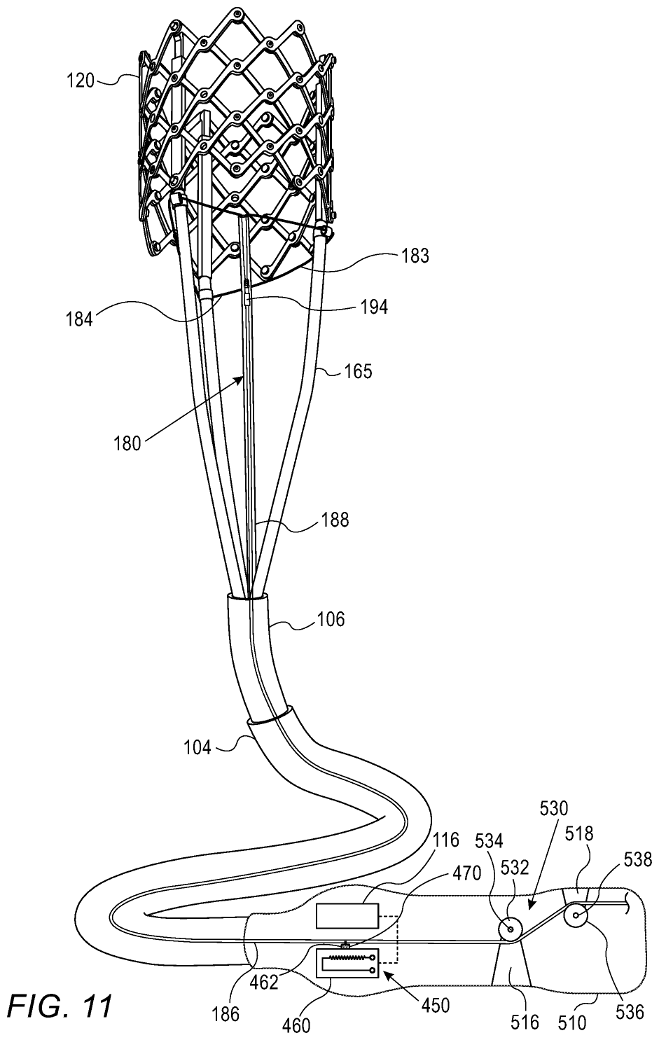

FIG. 11 shows yet another embodiment of a handle 510 comprising a diameter gauge 450. The handle 510 comprises a pulley assembly 530, which is similar to pulley assembly 430 with certain differences. The pulley assembly 530 comprises first and second pulleys 532 and 536, respectively. The first pulley 532 is mounted to any portion of the handle 510, and can be connected via a first pin 534 to a first pulley support member 516 of the handle 510. The second pulley 536 can be mounted to any portion of the handle 510, and may be laterally and/or axially offset from the first pulley 532. According to some embodiments, the second pulley 536 is connected via a second pin 538 to a second pulley support member 518 of the handle 510. The first and second pulleys 532 and 536 are freely rotatable about the first and second pins 534 and 538, respectively.

The main difference between the pulley assembly 530 and the pulley assembly 430, is that the second pulley 536 is devoid of a gear portion, and is therefore not engaged with a rack. As shown in FIG. 11, the re-compression member proximal segment 186 can be routed partially around the first pulley 532 and partially around the second pulley 536, extending beyond the second pulley 536, for example, in a proximal direction, instead of being configured to wrap there-around. According to some embodiments, the pulley assembly 530 may include one or more additional pulleys around which the re-compression member proximal segment 186 may be routed. The re-compression member proximal segment 186 can be attached to a pulling mechanism (not shown) at a location proximal to the second pulley 536, wherein the pulling mechanism is configured to either pull the re-compression member proximal segment 186 in a proximally oriented direction, or release the re-compression member proximal segment 186.

The pulley assembly 530 may be adjusted to apply a minimal tension magnitude Ts to the re-compression member 182 at all times, including in a released state, in a similar manner described above for the pulley assembly 430. According to other embodiments, a spring 220 is attached to the re-compression member proximal segment 186 according to any of the embodiments described above, in addition to the re-compression member proximal segment 186 being routed through the pulley assembly 530 (embodiments not shown).

The diameter gauge 450 comprises a displacement sensor 460, wherein at least one component of the diameter gauge 450 is coupled to the re-compression assembly 180 at a gauge coupling point 470, such that the displacement sensor 460 is operatively connected to the re-compression assembly 180.

The term “operatively connected”, as used herein, refers to any type of interaction between two components, wherein an action of a first component is effective to cause a reaction in the second components. For example, a displacement sensor 460 is operatively connected to the re-compression assembly 180 if an axial movement of a component of the re-compression assembly 180, such as the component comprising the gauge coupling point 470, is effective to cause the displacement sensor 460 to produce a corresponding signal (e.g., an electric signal or an optic signal).

According to some embodiments, as shown in FIG. 11, the displacement sensor 460 is operatively connected to the re-compression member proximal segment 186 at a gauge coupling point 470, and configured to generate a signal, wherein the magnitude of the signal is proportional to the position and/or axial displacement of the gauge coupling point 470.

According to some embodiments, the displacement sensor 460 comprises a potentiometer, and the diameter gauge 450 further comprise a wiper 462, coupled to the re-compression assembly 180 at the gauge coupling point 470. In the exemplary embodiment of FIG. 11, the wiper 462 is coupled to the re-compression member proximal segment 186 at the gauge coupling point 470. The wiper 462 is configured to move axially with the re-compression member proximal segment 186. The free end of the wiper 462, opposite to the gauge coupling point 470, is configured to contact the potentiometer 460, and its position relative to the potentiometer 460 affects the electric signal generated by the potentiometer 460. The position of the wiper 462 and its contact with the potentiometer 460, is directly proportional to the perimeter of the loop portion 183, which in turn is proportional to the perimeter of the prosthetic valve 120. Therefore, the diameter of the prosthetic valve 120, which can be derived from said perimeters, can be determined by measuring the electric signal generated by the potentiometer 460 which is in contact with the wiper 462.

As the re-compression member proximal segment 186, along with the gauge coupling point 470, is moved axially within the handle 510, the wiper 462 slides axially across the surface of the potentiometer 460, and a corresponding voltage may be transmitted to a control circuit (not shown). The control circuit may be embedded within the handle 510, and can include a processor for analyzing the voltage and deriving the valve expansion diameter accordingly.

It should be understood that a displacement sensor 460 is not limited to a potentiometer, and other displacement sensors, including linear displacement sensors, may be utilized. Exemplary alternative displacement sensors 460 can include a linear variable differential transformer (LDVT), an optical linear encoder, an optical sensor, a capacitive sensor, or any combination thereof. Angular displacement sensors may also be utilized in the same manner, for example to measure the angular or rotational movement of pulleys around which the re-compression members 182 extends, based on known correlations between such rotational movement and the axial displacement of the re-compression members 182.

According to some embodiments, the displacement sensor 460 is operatively coupled to the control unit via one or more wires or cables, or via a wireless communication link. The control unit can be configured to receive signals from the displacement sensor, representative of the axial movement of the re-compression member proximal segment 186. The control unit can be configured to continuously calculate the diameter of the prosthetic valve 120, based on measurement inputs provided by the displacement sensor 460.

According to some embodiments, the displacement sensor 460 is operatively coupled to a visual interface, such as a display 116. According to some embodiments, the displacement sensor 460 is operatively coupled to a display 116 via the control unit. The display 116 may comprise a digital screen, which may present numerical values indicative of the valve current diameter, as well as other icons, textual messages or graphical symbols. Additionally or alternatively, a visual interface may comprise LED lights 118, lamps or other visual elements, configured to provide the user with a visual indication of the current valve diameter. According to some embodiments, the control unit is configured to display the diameter of the prosthetic valve 120 on the display 116 in real-time, as the prosthetic valve 120 is expanded and/or compressed during an implantation procedure.

According to some embodiments, the control unit further comprises a memory. According to some embodiments, selected data, such as raw signal data or calculated data, may be stored in the memory. According to some embodiments, the control unit is configured to log data during the implantation procedure in the memory. According to some embodiments, the control unit is configured to transmit to a remote device, logged data from the memory, and/or real-time data.

According to some embodiments, the control unit is configured to provide an alert to an operator in the event of valve over-expansion within a native annulus. The alert may be an audible alert, a visual alert, a tactile alert, etc.

According to some embodiments, the control unit may be further configured to control the actuation arm assemblies 165 and/or the re-compression assembly 180, to expand and/or contract the prosthetic valve 120, according to pre-programmed expansion/contraction algorithms.

According to some embodiments, the control unit, and/or the display 116, may be provided as distinct components separated from the delivery apparatus 102, which can be operatively connected thereto, for example using wires or cables. According to some embodiments, the control unit, and/or the display 116, are configured to communicate wirelessly with the displacement sensor 460, such as via Bluetooth communication, radio waves, infrared signals, or other wireless communication protocols. According to additional embodiments, the control unit, and/or the display 116, are integrated within the handle 510. For example, a processor and other electrical components of a control unit, can be located within the handle 510, and the display 116 may be located on an exterior surface of the handle 510, such that it can be viewed by a clinician during the implantation procedure.

According to some embodiments, the diameter gauge 450 may be similarly utilized in conjunction with any other embodiments of handles disclosed hereinabove. For example, the diameter gauge 450 may be embedded within the handle 310, having the re-compression member proximal segment 186 coupled to the wiper 462 and to a spring 220. In another example, the diameter gauge 450 may be embedded within the handle 410, having the re-compression member proximal segment 186 routed through the pulley assembly 430 instead of the pulley assembly 530.

As mentioned above, tension applied to the re-compression member 182 may occasionally extend the length of the re-compression member 182 to a certain degree relative to a released state, or relative to its length under other pull force magnitudes that may be applied thereto. Such changes in the length of the re-compression member 182 may alter the position of the gauge coupling point 270, 370 or 470. This may, in turn, result in inaccuracies in valve diameter estimation.

According to some embodiments, the re-compression assembly further comprises a track member, extending through the re-compression shaft and attached, via a secondary loop, to the actuation arm assemblies 165, in a similar manner to that of the re-compression member 182. However, unlike the re-compression member 182, the tracking member is not configured to displace the actuation arm assemblies 165 in any direction, but rather to passively follow their displacements in the radial direction.

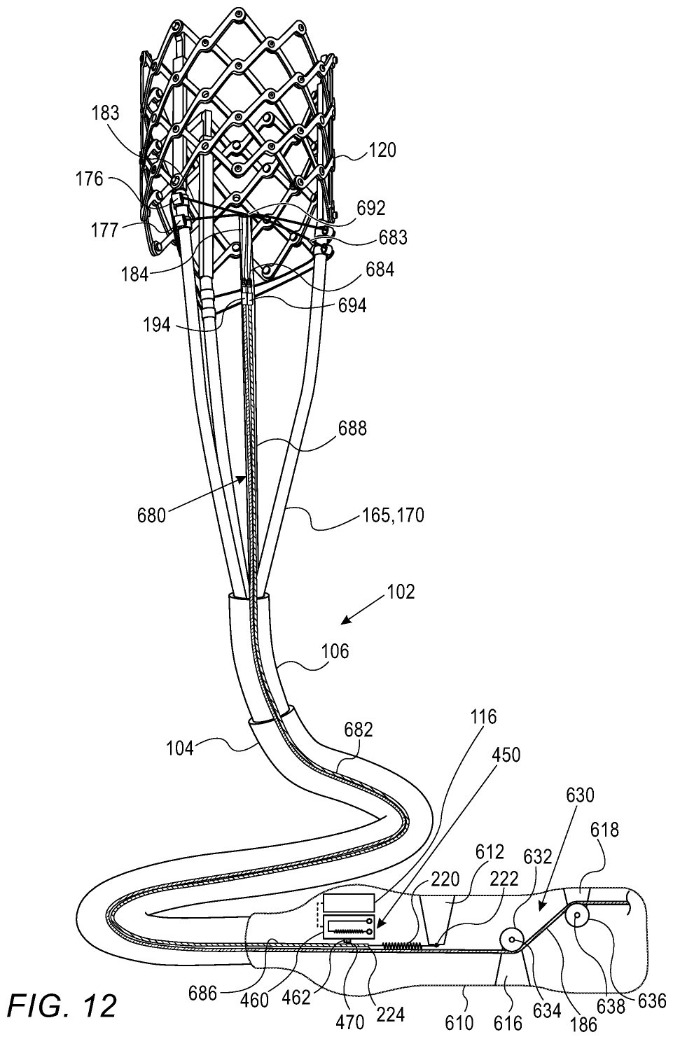

FIG. 12 shows a delivery apparatus 102 equipped with a re-compression assembly 680 and a handle 610, according to some embodiments. The re-compression assembly 680 is similar to the re-compression assembly 180, having the re-compression member 182 extending through a lumen of a re-compression shaft 688. However, the re-compression assembly 680 further comprises a track member 682, routed from the handle 610, through the re-compression shaft 688, toward the support sleeve distal ends 172.

The track member 682 may be provided in the form of a wire, a cable, a string and so on. According to some embodiments, the track member 682 can be made of the same materials as the re-compression member 182, and may be provided in the form of a wire, a cable, a string, and so on. According to some embodiments, the track member 682 comprises material which resists elongation in an axial direction, to a higher extent relative to the resistance to elongation of the re-compression member 182, for example, in a tensioned state of the re-compression assembly 680.

The track member 682 comprises a track member proximal segment 686 and a track member distal segment 684, which are the equivalents of the re-compression member proximal segment 186 and the re-compression member distal segment 184 described in any of the embodiments above. In some instances, the track member proximal segment 686 may be formed as a continuous extension of the track member distal segment 684. Alternatively, the track member proximal segment 686 and the track member distal segment 684 may be provided as separate components attached to each other, wherein either both segments are formed from the same materials having the same dimensions, or both are formed from the same materials but each is having different dimensions (e.g., one segment being thicker than the other), or each is formed from different materials but both are having similar dimension or dissimilar dimensions with respect to one another.

According to some embodiments, the track member distal segment 684 can be attached to the track member proximal segment 686 via a connector 694, which may be implemented according to any of the embodiments related to the connector 194.

According to some embodiments, each support sleeve 170 can include a secondary loop attachment member 177, which can be positioned adjacent the corresponding loop attachment member 176 of the same support sleeve 170. The secondary loop attachment member 177 may be implemented according to any of the embodiments described for the loop attachment member 176. Each secondary loop attachment member 177 may be axially distanced from the corresponding loop attachment member 176, either distal or proximal thereto. Alternatively or additionally, each secondary loop attachment member 177 may be angularly offset along the support sleeve 170, relative to the corresponding loop attachment member 176. For example, the secondary loop attachment member 177 and the loop attachment member 176 can be positioned at diametrically opposing sides of the respective support sleeve 170.

The track member 682 extends from the handle 610, shown in FIG. 12, through a lumen of the re-compression shaft 688, having a portion of its track member distal segment 684 extending distally from the re-compression shaft distal end 692, forming a secondary loop 683 connected to, and extending between, the actuator arm assemblies 165.

According to some embodiments, the secondary loop 683 is connected to, and extending between, the secondary loop attachment members 177, such that the secondary loop 683 is adjacent the loop portion 183, which extends between the loop attachment members 176. According to alternative embodiments, both the loop portion 183 and the secondary loop 683 may extend through the same loop attachment members 176.

According to some embodiments, both the track member 682 and the re-compression member 182 may extend, side-by-side, through the same lumen of the re-compression shaft 688. In alternative embodiments, the re-compression shaft 688 is a multi-lumen shaft, having each of the track member 682 and the re-compression member 182, extending through a separate lumen thereof.

According to some embodiments, a diameter gauge is attached, at the gauge coupling point, to the track member proximal segment 686 (instead of being attached to the re-compression member proximal segment 186), and is configured to provide real-time indication of the diameter of the prosthetic valve 120, based on axial position and/or axial translation of the gauge coupling point.

The handle 610 shown in FIG. 12 is similar to the handle 510, comprising a pulley assembly 630 which may be identical to the pulley assembly 530, with like numbers referring to like components, such that the re-compression member proximal segment 186 may be routed through the pulley assembly 630. Alternatively, the re-compression member proximal segment 186 may be routed through a pulley assembly 430, or may be connected to a pulling-mechanism, configured to apply or release a pull-force thereto, without having the re-compression member proximal segment 186 extend between any pulleys within the handle.

According to some embodiments, a minimal tension magnitude Ts' is applied, at all times, to the track member distal segment 684, and more specifically, to the secondary loop 683, configured to retain the secondary loop 683 in a minimally tensed state between the actuation arm assemblies 165, while allowing free expansion of the prosthetic valve 120 in a radial direction. According to some embodiments, the magnitude of the minimal tension magnitude Ts' applied to the secondary loop 683 is substantially identical to the magnitude of the minimal tension magnitude Ts applied to the loop portion 183. According to some embodiments, the magnitude of the minimal tension magnitude Ts' is different than the magnitude of the minimal tension magnitude Ts, for example due to a different axial position of the secondary loop 683 relative to the loop portion 183.

According to some embodiments, the handle 610 may further comprise a track spring 620, which may be identical in structure and function to the spring 220. The track spring 620 is attached to a spring support member 612 of the handle 610 via a spring first end 622, and to the track member proximal segment 686 via a spring second end 624. Contrary to the embodiments described and illustrated for the spring 220 in conjunction with FIGS. 8A-9, the spring 620 is configured to apply an axially oriented pull-force on the track member proximal segment 686 instead of the re-compression member proximal segment 186. The magnitude of the force applied by the spring 620 is sufficient to apply the minimal tension magnitude Ts' to the secondary loop 683.

The re-compression member 182 may be utilized to compress the valve 120, having its re-compression member proximal segment 186 attached to, and controllable by, a user controllable element, according to any of the embodiments described herein above. The track member 682, on the other hand, is not connected to the user controllable element, and therefore is not necessarily utilized to compress the valve 120. Rather, the track member 682 is configured to follow the change in valve diameter, having the secondary loop 683 configured to merely follow expansion or contraction of the prosthetic valve 120 in a similar manner to that described for loop portion 183, in any of the embodiments herein above.

Advantageously, since the maximal tension applied to the track member 682 is the minimal tension magnitude Ts′, which is substantially lower than the tension applied to the re-compression member 182 to compress the diameter of the prosthetic valve 120, the length of the track member 682 is not extended to the same extent as that of the re-compression member 182 in a tensioned state of the re-compression assembly 680.

According to some embodiments, the diameter gauge is coupled, at the gauge coupling point, to the track member proximal segment 686 (and not to the re-compression proximal segment 186).

In the exemplary embodiment shown in FIG. 12, a diameter gauge 450, comprising a displacement sensor 460, such as a potentiometer, is coupled to the track member proximal segment 686 at the gauge coupling point 470. More specifically, the wiper 462 is attached to the track member proximal segment 686 at the gauge coupling point 470, and is configured to interact with the potentiometer 460 in the same manner described and illustrated in conjunction with FIG. 11. Thus, the valve diameter may be derived from the axial movement of the gauge coupling point 470, having a corresponding indication shown, for example, in the display 116, in the same manner described and illustrated in conjunction with FIG. 11.

Advantageously, this configuration separates between the functionality of the re-compression member 182 and the functionality of the diameter gauge, such that while the re-compression member 182 is utilized to re-compress the prosthetic valve 120 as necessary, the diameter gauge follows such changes in diameter, without being affected by inaccuracies that may arise from axial elongation of the re-compression member 182 due to the pull-force applied thereto during tensioning states.

According to some embodiments, the track spring 620 may be further attached to the re-compression member proximal segment 186, thereby applying a similar basic tensioning force on both track member 682 and the re-compression member proximal segment 186 (embodiments not shown). Alternatively or additionally, a spring 220 may be attached to the re-compression member proximal segment 186, optionally in addition to the track spring 620 attached to the track member 682.

According to some embodiments, the re-compression assembly 680 may be similarly utilized with the track member proximal segment 686 attached to a dial pointing at indicator marks, in the same manner described and illustrated for the dial 254 and indicator marks 258 in conjunction with FIGS. 8A-8D, or in the same manner described and illustrated for the dial 354 and indicator marks 358 in conjunction with FIG. 9. In such embodiments, the track member proximal segment 686 may be attached to the track spring 620, and/or routed around pulleys of a pulley assembly, similar to the pulley assembly 430 or the pulley assembly 530.

While FIGS. 9-12 are demonstrated for a re-compression assembly 180, 680 having a loop portion 183 extending between loop attachment members 176, it will be clear that all of the configurations and embodiments illustrated and described in conjunction with FIGS. 9-12 can be used in combination with a re-compression assembly 180 having a loop portion 183 circumscribing the prosthetic valve 120, similar to the configurations illustrated in FIGS. 8C-8D.

Specifically, a re-compression assembly 680 comprising a re-compression member 182 with a loop portion 183 extending between loop attachment members 176, as illustrated in FIG. 12, can similarly comprise, in certain embodiments, a re-compression assembly 180 having a loop portion 183 circumscribing the prosthetic valve 120. In such embodiments, the track member 682 may be similarly provided with a secondary loop 683 also circumscribing the prosthetic valve 120. All other embodiments described in conjunction with FIG. 12 are similarly applicable with the re-compression assembly 680 having the loops 183 and 683 circumscribing the prosthetic valve 120.

While not explicitly illustrated, additional embodiments of a re-compression assembly 680 may include a re-compression member 182 having a loop portion 183 extending between loop attachment members 176 and configured to apply sufficient tension so as to compress the prosthetic valve 120, used in combination with a track member 682 having a secondary loop 683 circumscribing the prosthetic valve 120 and configured to merely track the change in perimeter of the prosthetic valve 120. Alternatively, embodiments of a re-compression assembly 680 may include a re-compression member 182 circumscribing the prosthetic valve 120 and configured to apply sufficient tension so as to compress the prosthetic valve 120, used in combination with a track member 682 having a secondary loop 683 extending between loop attachment members 176 and configured to merely track the change in perimeter of the prosthetic valve 120.

According to some embodiments, a diameter gauge according to any of the embodiments of the current disclosure is operatively coupled to the digital display 116 or to the LED lights 118. According to some embodiments, a diameter gauge is operatively coupled to the digital display 116 or the LED lights 118 via the control unit. The digital display 116 may comprise a digital screen, which may present numerical values indicative of the current diameter of the prosthetic valve 120. The digital display 116 may similarly display other icons, textual messages and/or graphical symbols. Additionally or alternatively, LED lights 118, lamps or other visual elements, can be configured to provide the user with a visual indication regarding the diameter of the prosthetic valve 120. According to some embodiments, the control unit is configured to display the diameter of the prosthetic valve 120 on the digital display 116 in real-time, as the prosthetic valve 120 is expanded and/or compressed during an implantation procedure.

According to some embodiments, the control unit further comprises a memory member, and selected data, such as raw signal data or calculated data, can be stored in the memory member. A memory member may include a suitable memory chip or storage medium such as, for example, a PROM, EPROM, EEPROM, ROM, flash memory, solid state memory, or the like. A memory member can be integral with the control unit or may be removably coupled to the control unit. According to some embodiments, the control unit is configured to log data during the implantation procedure in the memory member. According to some embodiments, the control unit is configured to transmit logged data from the memory member, and/or real-time data, to a remote device.

According to some embodiments, the control unit is configured to provide an alert to an operator in the event that the diameter of the prosthetic valve 120 exceeds a predefined threshold. The alert can be an audible alert, a visual alert, a tactile alert, etc.

According to some embodiments, the control unit can be further configured to control the actuation arm assemblies 165 to expand the prosthetic valve 120 according to pre-programmed expansion algorithms.

According to some embodiments, the control unit and or the display 116 may be provided as distinct components, separated from the delivery apparatus 102, and operatively connected thereto, for example using wires or cables. According to additional embodiments, the control unit and/or the display 116 can be formed integrally with the handle. For example, a processor and other electrical components of a control unit can be located within the handle, and the display 116 can be located on an exterior surface of the handle, as shown in FIG. 1, such that it can be viewed by a clinician during the implantation procedure.

According to some embodiments, an axially stationary component of the delivery assembly 100, configured to maintain an affixed axial position relative to the outflow end 123 during expansion or compression of the prosthetic valve 120, comprises at least one reference radiopaque marker 882, and an axially movable component of the re-compression assembly 180, configured to be axially movable relative to the outflow end 123 during expansion or compression of the prosthetic valve 120, comprises at least one indicator radiopaque marker 880.

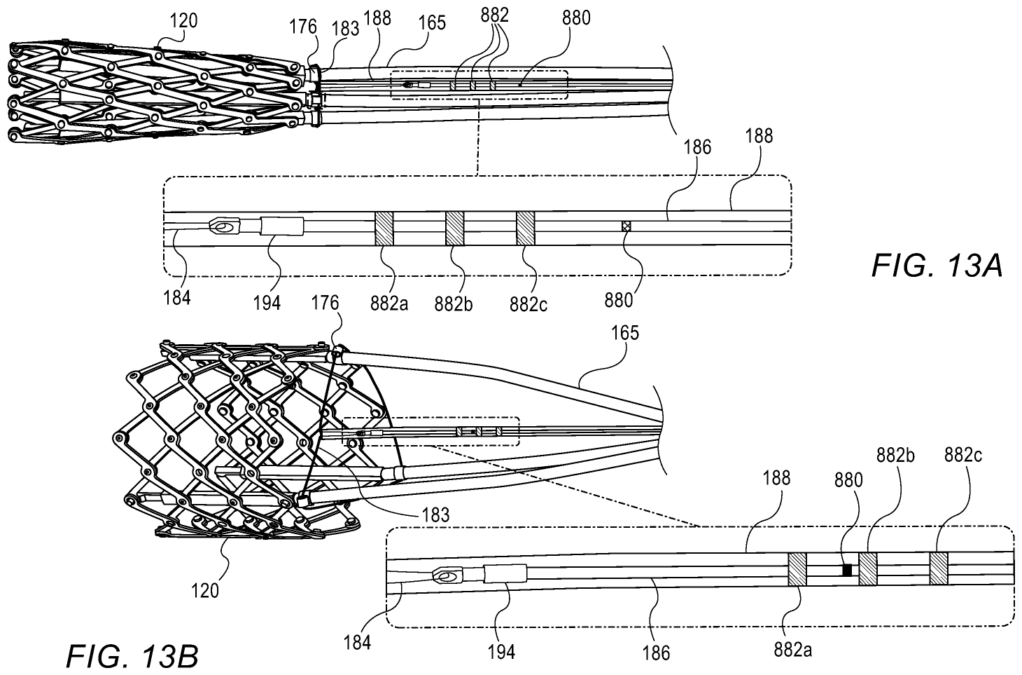

According to some embodiments, as shown in FIGS. 13A-13B, the axially stationary component is the re-compression shaft 188, comprising at least one reference radiopaque marker 882 around its outer surface, and the axially movable component is the re-compression member proximal segment 186, comprising at least one indicator radiopaque marker 880 around its outer surface. Each reference radiopaque marker 882 and each indicator radiopaque marker 880 can be implemented according to any of the embodiments described herein above for radiopaque markers 196 in conjunction with FIG. 6A.

According to some embodiments, the at least one indicator radiopaque marker 880 is configured to be visually distinguishable from the at least one reference radiopaque marker 882, for example by having different dimensions. In the exemplary embodiments of FIG. 13A, the indicator radiopaque marker 880 is disposed around the outer surface of the re-compression member proximal segment 186, which is thinner than the re-compression shaft 188 it is disposed in, resulting in an indicator radiopaque marker 880 which is relatively smaller than each of the reference radiopaque marker 882. In some applications, the length of the indicator radiopaque marker 880 may be different than the length of the reference radiopaque markers 882.

According to some embodiments, the re-compression shaft 188 comprises a radiolucent material or has a cut-out window, enabling the at least one indicator radiopaque marker 880 to be visible there-through under fluoroscopy.

FIG. 13A shows the prosthetic valve 120 in a compressed state, while FIG. 13B shows the valve 120 in an expanded state. The re-compression shaft 188 may be maintained coupled to the handle 110 at a predetermined position, having a fixed length thereof extending toward the valve 120, such that the position of any portion of the re-compression shaft 188, including the reference radiopaque marker 882 disposed around its outer surface, maintain the same axial position relative to the outflow end 123 of the valve 120 during expansion (e.g., for the compressed state of FIG. 13A to the expanded state of FIG. 13B) or compression (e.g., for the expanded state of FIG. 13B to the compressed state of FIG. 13A) of the prosthetic valve 120.

During valve expansion, as shown in FIG. 13B, the loop portion 183 of the re-compression member distal segment 184 expands therewith, axially translating the re-compression member proximal segment 186, as well as the indicator radiopaque marker 880 disposed thereon, in a distally oriented direction (i.e., toward the outflow end 123) relative to its position in the compressed state of FIG. 13A, and relative to the reference radiopaque markers 882.

As mentioned above, axial translation of the re-compression member proximal segment 186 is proportional to the perimeter of the loop portion 183, which in turn is proportional to the diameter of the prosthetic valve 120. Thus, the at least one reference radiopaque markers 882 may serve as a “scale”, and the indicator radiopaque marker 880 can serve as a “dial” relative to the “scale”, indicative of the diameter of the prosthetic valve 120. Accordingly, the re-compression assembly 180 provided in a delivery assembly 100 to facilitate re-compression of a prosthetic valve 120 when required, can be advantageously further utilized to serve as a real-time monitoring aid for prosthetic valve diameter during expansion or compression thereof, based on the alignment of at least one indicator radiopaque marker 880 relative to reference radiopaque markers 882 under fluoroscopy. In other words, the axial position of the one indicator radiopaque marker 880 relative to the reference radiopaque markers 882 is indicative of the diameter of the prosthetic valve 120.

In applications of the re-compression shaft 188 comprising three reference radiopaque markers 882 or more, the reference radiopaque markers 882 may be equally spaced from each other. Alternatively, at least some of the reference radiopaque markers 882 may be space with unequal distances.

The re-compression shaft 188 can include a plurality of reference radiopaque markers 882, such as the three reference radiopaque markers 882a, 882b and 882 shown in FIGS. 13A-13B, each corresponding to a specific diameter of the prosthetic valve 120. In the illustrated example, the most proximal reference radiopaque marker 882a can correspond to a first expanded diameter, such as 27 mm. The intermediate reference radiopaque marker 882b can correspond to a second expanded diameter, larger than the first diameter, such as 28 mm. The most distal reference radiopaque marker 882c can correspond to a third (potentially maximal) expanded diameter, larger than the second diameter, such as 29 mm. Alignment of the indicator radiopaque marker 880 with any of the reference radiopaque markers 882 may be indicative of the valve diameter associated with the reference radiopaque marker 882. Positioning of the indicator radiopaque marker 880 between any couple of reference radiopaque markers 882, as shown in FIG. 13B, may be indicative of an expansion diameter between the two diameters associated with the respective reference radiopaque markers 882. Similarly, an indicator radiopaque marker 880 positioned distal to the most distal reference radiopaque marker 882c may indicate an expansion diameter exceeding a maximal value.

While three reference radiopaque markers are shown in the exemplary embodiment illustrated in FIGS. 13A-13B, it will be clear that any other number of reference radiopaque markers 882 is contemplated. For example, more than three reference radiopaque markers 882 may be utilized to provide a higher resolution of the “scale” provided thereby. Alternatively, a single reference radiopaque marker 882 may be provided, for example to serve only as a maximal threshold value, such that if the indicator radiopaque marker 880 translates distally to the single reference radiopaque marker 882, it may be indicative of an expansion diameter that exceeds a maximal threshold value (embodiment not shown).

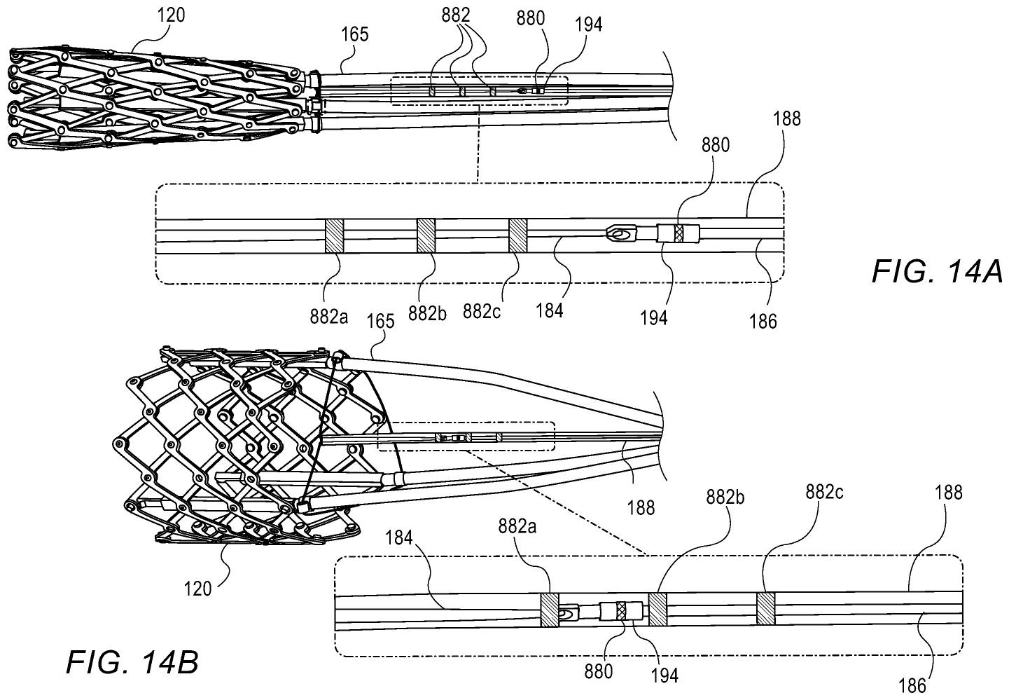

According to some embodiments, as shown in FIGS. 14A-14B, the axially stationary component is the re-compression shaft 188, comprising at least one reference radiopaque marker 882 around its outer surface, and the axially movable component is the re-connector 194, comprising at least one indicator radiopaque marker 880 around its outer surface.

FIGS. 14A and 14B show views similar to those shown in FIGS. 13A and 13B respectively, wherein the re-compression mechanism 180 is identical to any of the embodiments described in conjunction with FIGS. 13A-13B, except that the axially movable component comprising the indicator radiopaque marker 880 is the connector 194. If the connector 194 is a releasable connector, the radiopaque marker may be disposed around either one of the proximal connector element 193, the distal connector element 195, or both.

FIGS. 13A-14B show exemplary configurations of a loop portion 183 of the re-compression member distal segment 184 threaded through eyelet-shaped loop attachment members 176, as elaborated in conjunction with FIGS. 8A-8B. These configurations may be advantageous as both the connector 194 and the re-compression member proximal segment 186 are positioned proximal to the outflow end 123 of the valve 120 at all times, along with the indicator radiopaque marker 880, disposed on either one of the aforementioned components. Thus, the indicator radiopaque marker 880, as well as the respective reference radiopaque markers 882, are positioned proximal to the valve outflow end 123, so as to be visible throughout expansion or compression of the valve 120 under fluoroscopy without being potentially obstructed by the frame 126.

FIGS. 15A and 15B show views similar to those shown in FIGS. 13A and 13B, respectively, wherein the re-compression mechanism 180 is identical to any of the embodiments described in conjunction with FIGS. 13A-14B, except that the loop portion 183 of the re-compression member distal segment 184 circumscribes the prosthetic valve 120 instead of being threaded through loop attachment members 176 of the actuator arm assemblies 165. The frame 126 and other components of the prosthetic valve 120 are removed from view in the zoomed-in portions of FIGS. 15A-15B for clarity.

In some applications, the loop portion 183 can enter into a circumferential sleeve 830 through a circumferential sleeve opening 833. The circumferential sleeve 830 can be disposed around the frame 126 and attached thereto by gluing, suturing/stitching, and the like. For example, the circumferential sleeve 830 can be sutured to a plurality of junctions 130 and/or struts 127 of the frame 126. While the loop portion 183 is shown to extend through a circumferential sleeve 830 in FIGS. 15A-15B, it will be clear that the loop portion 183 of the re-compression member distal segment 184 can similarly extend through a sleeve 130 attached to, or integrally formed with, a skirt such as an outer skirt 137, in the same manner described and illustrated in conjunction with FIGS. 7A-7C, or it can be looped directly over the valve 120 without extending through any type of sleeve, in the same manner described an illustrated in conjunction with FIGS. 5B-6B.

The configuration shown in FIGS. 15A-15B illustrates a re-compression mechanism 180 equipped with a releasable connector 194, disposed within a guide member 840, similar to the embodiments described and illustrated in conjunction with FIGS. 7A-7C. Nevertheless, it will be clear that the embodiments described in conjunction with FIG. 15A-15B are similarly applicable to configurations of a re-compression mechanism 180 which may be equipped with a non-releasable connector 194, and does not necessarily extend through a guide member 840, in the same manner described an illustrated in conjunction with FIGS. 5B-6B. For configurations that do include a guide member 840, it is preferable for the indicator radiopaque marker 880 and the reference radiopaque markers 882 to be positioned throughout the range of expansion diameters, proximal to the outflow end 123 of the valve 120. For example, the indicator radiopaque marker 880 and the reference radiopaque markers 882 are illustrated proximal to the guide member proximal end 844 both in the compressed state and the expanded state shown in FIGS. 15A and 15B, respectively, thereby facilitating visibility thereof under fluoroscopy without being potentially obstructed by the frame 126.

In some applications, as shown in FIGS. 16A and 16B which are equivalent to the views shown in FIGS. 15A and 15B, respectively, the reference radiopaque markers 882, such as markers 882a, 882b and 882c, are disposed over the outer surface of the guide member 840 instead of the outer surface of the re-compression shaft 188. Since the guide member 840 is attached to the frame 126, extending distally from the outflow end 123, the struts 127 may mask such reference radiopaque markers 882 and/or the indicator radiopaque marker 880, due to their own inherent radiopacity. In some applications, the radiodensity of the reference radiopaque markers 882 and/or the indicator radiopaque marker 880 is higher than that of the struts 127 or other components of the prosthetic valve 120, so as to enable the radiopaque markers 880, 882 to be visibly distinguishable, under fluoroscopy, from the frame 126 or other components of the valve 120.

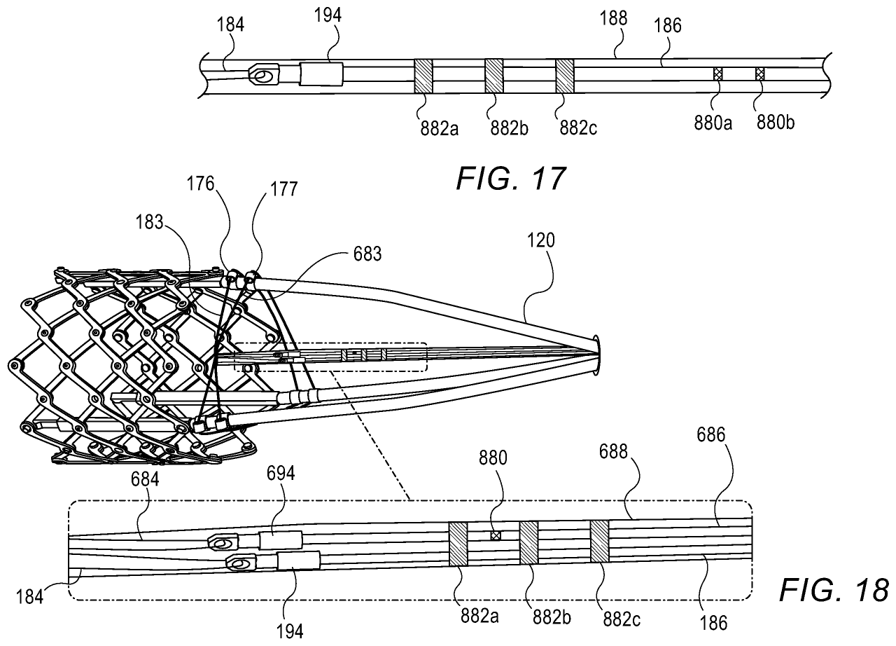

According to some embodiments, more than one indicator radiopaque marker 880 may be utilized. For example, FIG. 17 shows a zoomed in view of a portion of the re-compression mechanism 180, which can be implemented in combination with any of the configurations described and illustrated in conjunction with FIGS. 13A-16B, wherein the re-compression shaft 188 comprises three reference radiopaque markers 882a, 882b, 882c, and the re-compression member proximal segment 186 comprises two indicator radiopaque marker 880a, 880b. The distance between the plurality of indicator radiopaque markers 880 can be different than the distance between equi-spaced (or otherwise spaced) reference radiopaque markers 882. For example, setting the distance between indicator radiopaque markers 880a and 880b to be half of the distance between any two adjacent reference radiopaque markers 882, may enhance the potential resolution of the indicated diameter.

As mentioned above with respect to embodiments described and illustrated in conjunction with FIGS. 8A-12, tension applied to the re-compression member 182 may occasionally extend the length of the re-compression member 182 to a certain degree relative to a released state, or relative to its length under other pull force magnitudes that may be applied thereto. Such changes in the length of the re-compression member 182 may alter the position of the indicator radiopaque marker 880. This may, in turn, result in inaccuracies in valve diameter estimation.

Thus, any of the embodiments described and illustrated in conjunction with FIGS. 13A-17 may be used in combination with a handle comprising a spring 220 or equivalent thereof, connected to the re-compression member proximal segment 186 according to any of the embodiments described and illustrated in conjunction with FIGS. 8A-9. Similarly, any of the embodiments described and illustrated in conjunction with FIGS. 13A-17 may be used in combination with a handle comprising a pulley assembly 430 or 530 according to any of the embodiments described and illustrated in conjunction with FIGS. 10A-10B or 11, respectively.

In further applications, any of the embodiments described and illustrated in conjunction with FIGS. 13A-14B and/or 17 may be used in combination with a delivery apparatus 102 equipped with a re-compression assembly 680 according to any of the embodiments described and illustrated in conjunction with FIG. 12. For example, FIG. 18 illustrates re-compression assembly 680 provided with both a re-compression member 182 and a track member 682, similar to any of the embodiments described and illustrated in conjunction with FIG. 12. As illustrated, the re-compression shaft 688 can include the reference radiopaque markers 882, and the track member proximal segment 686 (or in alternative embodiments, the connector 694) can include the indicator radiopaque marker 880. In such embodiments, the re-compression member 182 can be utilized to facilitate valve compression when required, while the position of the indicator radiopaque marker 880 relative to the reference radiopaque markers 882 of the re-compression shaft 688 may provide a real-time indication of the valve diameter, as elaborated hereinabove.

While a plurality of reference radiopaque markers 882, such as three markers 882a, 882b and 882c, are illustrated, it should be appreciated that a single reference radiopaque marker 882 can be similarly utilized in any of the embodiments described in conjunction with in FIGS. 13A-18. A single reference radiopaque marker 882 can represent a critical expansion diameter of interest, such as a maximal allowable expansion diameter, such that a relative position of the indicator radiopaque marker 880 relative to the reference radiopaque marker 882 may be indicative of valve over-expansion.

According to another aspect of the invention, there is provided a method of providing real-time estimation of the expansion diameter of a prosthetic valve 120, based on a relationship between the expansion diameter and a dimensionless parameter. The dimensionless parameter is either an opening angle of the prosthetic valve 120, or the aspect ratio between the length and diameter of the prosthetic valve 120, at each expansion diameter.

Frames of prosthetic valves 120 include a plurality of cells 135, defined between sections of the struts 127 intersecting at junctions 130. The shape of each cell 135, and its dimensions in different directions, vary during expansion or retraction of the prosthetic valve 120. The prosthetic valve 120 comprises a plurality of cells, such that the change in dimensions, for example, in the longitudinal and lateral directions, of cells 135, reflects on a change in the length and diameter of the prosthetic valve 120 as well.

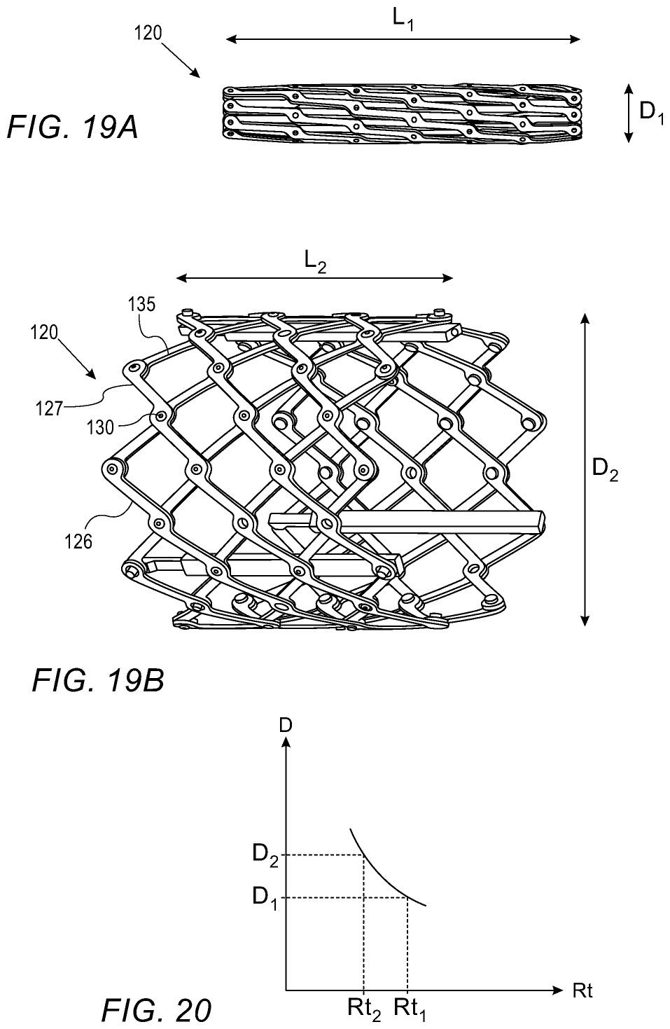

FIGS. 19A-19B show a mechanically expandable prosthetic valve 120 in a compressed state and an expanded state, respectively. The exemplary prosthetic valve 120 shown in FIGS. 19A-19B comprises struts 127 arranged in a lattice-type pattern, interconnected at hinged junctions 130 to form substantially diamond-shaped cells 135. In the crimped or compressed state shown in FIG. 19A, the cells 135 have a maximal axial length and minimal lateral width, such that the prosthetic valve 120 has a maximal length Li and a minimal diameter Di. In the expanded state shown in FIG. 19B, the cells 135 are stretched sideways (e.g. following rotation at the hinged junctions 130), forming substantially diamond shaped cells. As a result, the prosthetic valve 120 has a length L₂ which is shorter than L₁, and a minimal diameter D₂ which is smaller than D₁. While a specific type of mechanically expandable prosthetic valve 120 is shown in FIGS. 19A-19B, other valve types, that may include other cell shapes, are contemplated.

The aspect ratio Rt of the frame 126 can be defined as the ratio of the frame length L to the frame diameter D. The aspect ratio Rt is altered during expansion or compression of the prosthetic valve 120. For example, the aspect ratio in a compressed state Rt₁ is defined as L₁/D₁, and the aspect ratio in a compressed state Rt₂ is defined as L₂/D₂. FIG. 20 shows an exemplary curve representing the relationship between aspect ratio Rt and expansion diameter D for certain configurations. As shown, the aspect ratio Rt may have a different value for each expansion diameter D. While a specific non-linear relationship is shown in FIG. 20, it will be clear that other non-linear or linear relationships may be applicable.

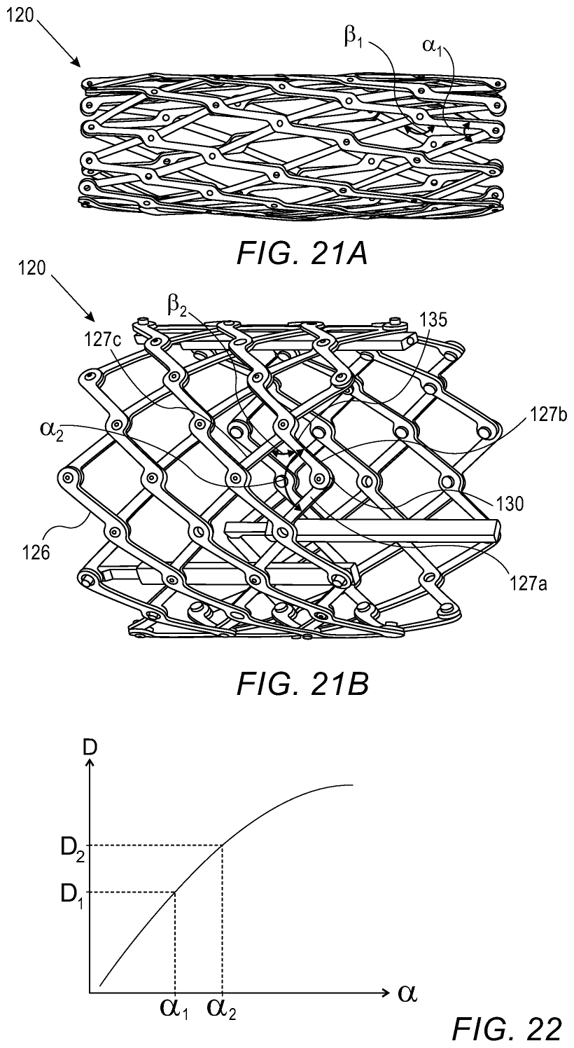

FIGS. 21A-21B illustrate a mechanically expandable prosthetic valve 120 in a partially expanded state and a fully expanded state, respectively, showing exemplary opening angles of the frame 126. An opening angle can be defined between any couple of struts 127 that intersect at a junction 130, wherein the angle varies during expansion or compression of the valve 120. Various types of opening angles may be defined, depending on the orientation of the selected opening angle. For example, an opening angle α oriented at a longitudinal direction of the valve 120 can increase, for example from the acute angle α₁ shown in FIG. 21A, to a larger, potentially obtuse (or at least, less acute) angle α₂ shown in FIG. 21B. An exemplary opening angle α is shown in FIGS. 21A-21B between intersecting struts 127a and 127b.

Similarly, an opening angle β oriented at a circumferential direction of the valve 120 can decrease, for example from the obtuse angle β₁ shown in FIG. 21A, to a smaller, potentially acute (or at least, less obtuse) angle β₂ shown in FIG. 21B. An exemplary opening angle β is shown in FIGS. 21A-21B between intersecting struts 127b and 127c. In the case of diamond or rhombus-shaped cells 135, angles α and β are complementary angles, meaning that each type of an opening angle can be easily derived from the complementary angle. Thus, any reference to a method of acquiring an opening angle α applies with equal force to acquiring an opening angle β.

FIG. 22 shows an exemplary curve representing the relationship between an opening angle α and expansion diameter D for certain configurations. As shown, the opening angle α may have a different value for each expansion diameter D. While a specific relationship of an opening angle α, increasing along with the expansion diameter D, is shown in FIG. 22, it will be clear that other types of relationships, including that of an opening angle β decreasing while the expansion diameter D increases, are contemplated. While a specific type of mechanically expandable prosthetic valve 120 is shown in FIGS. 21A-21B, other valve types, that may include other cell shapes, are contemplated.

Prosthetic implantation procedures are usually performed under fluoroscopy, wherein the frame 126 of the prosthetic valve 120 is radiopaque and visible on an external monitor. As disclosed herein, a known relationship between a dimensionless parameter (i.e., the aspect ratio Rt or the opening angle α) and expansion diameter D, for a desired range of expansion diameters D of a prosthetic valve 120, can be exploited to derive the expansion diameter D, or a close approximation thereof, during fluoroscopy imaging of the frame 126.

According to some embodiments, there is provided a method including the steps of (1) acquiring at least one image of the frame 126 of a prosthetic valve 120, (2) deriving a dimensionless parameter, from the at least one image, (3) associating a value of an expansion diameter D of the prosthetic valve with the dimensionless parameter, and (4) providing an indication (e.g., a visual indication) of the expansion diameter D of the prosthetic valve 120.

According to some embodiments, the dimensionless parameter in steps (2) and (3) of the method is the opening angle α (or β) between two intersecting struts of the frame 126.

According to some embodiments, the dimensionless parameter in steps (2) and (3) of the method is the aspect ratio Rt of the length of the frame to the width of the frame.

The terms “diameter of the prosthetic valve”, “diameter of the frame”, “valve diameter” and “expansion diameter”, as used herein, are interchangeable.

According to some embodiments, the step of imaging the frame 126 comprises acquiring at least one angiogram X-ray image of the frame 126. According to some embodiments, the step of imaging the frame 126 comprises acquiring at least one live fluoroscopy image of the frame 126. According to some embodiment, the at least one acquired image of the frame 126 is transmitted to a data control unit, which comprises a central processing unit (CPU). The data control unit is configured to identify information data within the at least one acquired image, such as to identify or detect the radiopaque frame 126.

According to some embodiments, the data control unit is configured to obtain parameters representing a length and a width of the frame 126, wherein the width being representative of the diameter of the frame 126. The length and width of the imaged frame 126 may be assigned values in any unit, including number of pixels in the image. An aspect ratio Rt is then calculated by the data control unit, dividing the length value by the width value. Alternatively or additionally, the angular position of intersecting struts 127 can be used to derive the angle there-between.

According to some embodiments, the data control unit further comprises a memory. The information of the at least one acquired image, including the measured length and width of the frame 126, angular position of intersecting strut 127, and calculated/derived aspect ratio Rt or opening angle α, β, may be stored in the memory.

According to some embodiments, known relationship between different aspect ratios Rt and valve expansion diameters D, and/or between opening angles α, β and valve expansion diameters D, are stored in the memory. The numerical value of the expansion diameter D of the frame 126 may be derived from the aspect ratio Rt and/or the opening angle α, β, based on any of: mathematical formulas, graphs, and/or tables, that may be stored in the memory. According to some embodiments, the step of providing a visual indication of the expansion diameter comprises visualization of the expansion diameter on a digital screen, as: a numerical value, an icon or other graphical symbol, a textual message, or any combination thereof.

Advantageously, the proposed method does not require the use of calibration components, such as calibration rulers that include radiopaque markings, to derive the expansion diameter. There is no need to directly measure the magnitude (e.g., the numerical value) of the length and/or diameter of the valve 120, as well as specific dimensions of struts 127, since the method relies on measurement of a dimensionless parameter (i.e., the aspect ratio Rt or the opening angle α, β), from which the expansion diameter may be derived, based on known relationships there-between.

A further advantage conferred by the delivery assemblies and the methods disclosed herein, is that they enable continuous real-time diameter monitoring, thereby providing valuable feedback to the clinician with respect to the valve expansion within the native anatomy. This valuable information may assist in preventing, or at least reducing, potential trauma to a tissue (e.g., the annulus). The clinician can continuously readjust the diameter of the prosthetic valve 120 as necessary, until the prosthetic valve 120 is expanded to a diameter that best fits the native annulus. For example, a diameter which is sufficient to anchor the prosthetic valve 120 in place against the surrounding tissue, with little or no paravalvular leakage, and without over-expanding the prosthetic valve 120 so as to avoid, or reduce the risk of, native annulus rupture.

According to another aspect of the invention, there is provided a prosthetic valve which includes a frame belt circumscribing at least a portion of the frame in an expanded state, wherein the frame belt includes at least one expansion force indicator, and preferably a plurality of expansion force indicators, configured to provide an indication of the circumferential force applied by the prosthetic valve 120, during expansion thereof, to the frame belt. According to some embodiments, the expansion force indicators are radiopaque-marked expansion force indicators, configured to provide visual indication (for example, under fluoroscopy) of the circumferential force applied by the prosthetic valve 120, during expansion thereof, to the frame belt.

As mentioned above, the native anatomy against which the prosthetic valve 120 is expanded, may exert responsive radial forces against the prosthetic valve 120 in an opposite direction. Thus, the diameter of the prosthetic valve 120 is correlated to a balance between outwardly oriented expansion forces applied by the valve 120 to the surrounding anatomy, and inwardly oriented responsive forces applied by the surrounding anatomy to the valve 120. Valve over-expansion may be defined as a situation in which the valve exerts excessive radial forces on the surrounding anatomy, resulting in potential damage to the tissue or even annular rupture. Assuming that the relationship between radial force and circumferential force is known for the specific valve type, the valve expansion force can be derived from the circumferential stress imparted on the frame belt by the valve, detectable by a change in a state of the expansion force indicators.

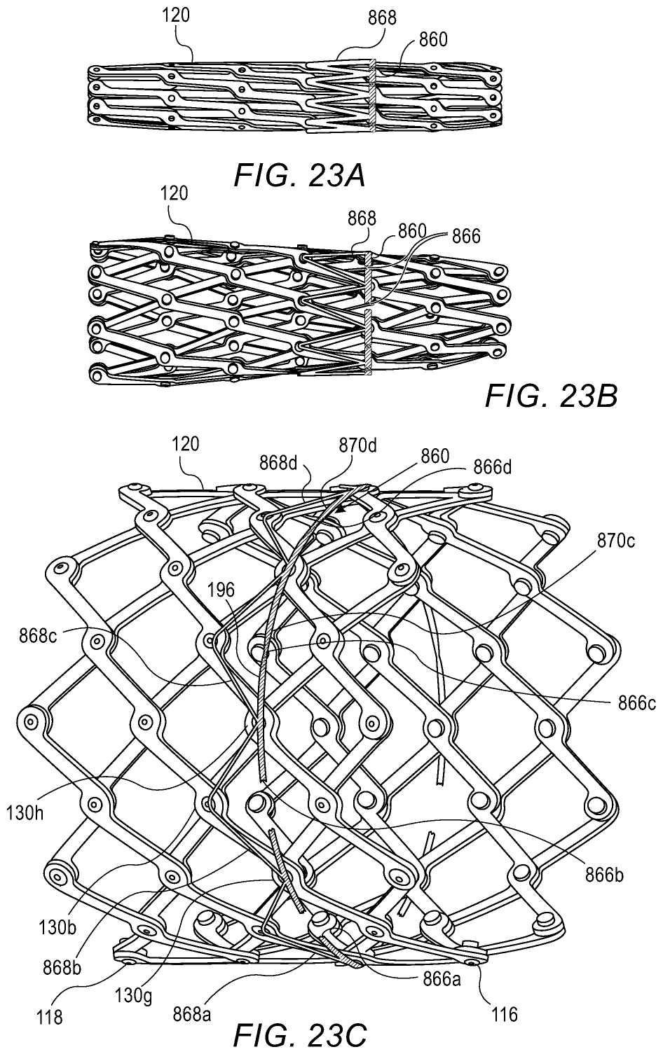

FIGS. 23A, 23B and 23C show a prosthetic valve 120 provided with a frame belt 860, in a compressed state, a partially expanded state, and a fully expanded state, respectively, according to some embodiments. The frame belt 860 comprises at least one frame belt diameter indicator 866, configured to change a state thereof when a force exceeding a specific magnitude is applied thereto by the frame 126, during expansion thereof. The change in the state of the at least one frame belt diameter indicator 866 is visually distinguishable under fluoroscopy.

In some applications, the at least one frame belt diameter indicator comprises a separation zone 866, and the visually distinguishable state comprises a transition of the separation zone 866 from an intact state to a separated state. Specifically, a separation zone 866 can comprise a radiopaque marker, according to any embodiment disclosed above for radiopaque markers 196 or 880, such that a separated separation zone 866 may be visible under fluoroscopy as a discontinuation of a radiopaque-marked region, which was visible as a continuous zone prior to separation thereof.

The term “disrupted” or “separated”, as used herein, are interchangeable, and refer to being torn, broken, or otherwise disconnected.

In some applications, the at least one radiopaque expansion force indicator comprises a geometrical feature 866 with a shape distinguishable from it neighboring zone, and the visually distinguishable state comprises a translation of the geometrical feature 866 from a first zone to a second zone. Specifically, a geometrical feature 866 can comprise a radiopaque marker, according to any embodiment disclosed above for radiopaque markers 196 or 880, such that spatial translation thereof from a first zone to a second zone may be visible under fluoroscopy. In some variants of the application, the first zone can include a radiopaque zone, configured to hide or mask the geometrical features 866 disposed behind it or within a lumen thereof, from visibility under fluoroscopy, and the second zone can include an exposed (or otherwise radiolucent) zone, in which the geometrical features 866 may be visible under fluoroscopy. In some variants of the application, the first zone can include a first orientation of a portion of the frame belt 860, defined between at least two geometrical features 866, and the second zone can include a second orientation of a portion of the frame belt 860, defined between at least two geometrical features 866, wherein the second orientation is angled, and is potentially perpendicular relative to, the first orientation. In some variants of the application, the first zone can include a first spatial position of a geometrical features 866 relative to a reference radiopaque marker, and the second zone can include a second spatial position of a geometrical features 866 relative to the reference radiopaque marker, wherein the first spatial position and the second spatial position are defined on opposite sides of the reference radiopaque marker (e.g., proximal and distal to the reference radiopaque marker).

In an exemplary embodiment illustrated in FIG. 23A, a frame belt 860 circumscribes the valve 120, wherein the frame belt 860 is intact as long as the valve 120 is not expanded to a diameter that applies a first critical tensioning force thereto. According to some embodiments, the frame belt comprises expandable portions 868, configured to circumferentially expand along with the frame 126 of the prosthetic valve 120, and base portions 870 provided with separation zone 866. Disruption of the separation zone 866 results in separation between sections of the base portions 870 on both sides thereof. Disruption of a separation zone 866 may occur upon application of a tensioning force sufficient facilitate such separation. The separation facilitating tensioning force may be applied to the separation zone 866 by the prosthetic valve 120 during expansion thereof.

The frame belt 860 illustrated in the exemplary embodiment of FIGS. 23A-23C includes expandable portions 868 in the form of struts connected at junctions to corresponding base portions 870. While resulting triangular cells are shown in FIGS. 23A-23C, it will be clear that any other form is applicable, as long as the expandable portions 868 are expandable without being disrupted, when disposed over, or attached to, the frame 126, and as long as at least one base portion 870 includes a separation zone 866 that may be disrupted or separated, upon application of a sufficient tensioning force applied thereto by the expansion of the frame 126.

The frame belt 860 may be disposed around the prosthetic valve 120, such that upon application of a tensioning force exceeding a first critical value, as illustrated for example in FIG. 23B, at least one separation zone 866 is disrupted, such as by being broken, torn, disconnected, decoupled, and the like. Further expansion of the prosthetic valve 120 may result in separation of additional separation zones 866. For example, FIG. 23C shows a frame belt 860 with disrupted separation zones 866a and 866b, while separation zones 866c and 866d remain intact.

In some applications, at least some apices and/or junctions of the frame belt 860, such as apices of expandable portions 868, and/or junctions connecting the expandable portions 868 with base portions 870, may be coupled to junctions 130 of the prosthetic valve 120, to facilitate simultaneous expansion of the expandable portions 868 with the frame 126.

While the frame belt 860 shown in FIGS. 23A-23A is disposed over the entire circumference of the valve 120, it will be appreciated that in some applications, the frame belt 860 can be disposed over a portion of the valve's circumference.

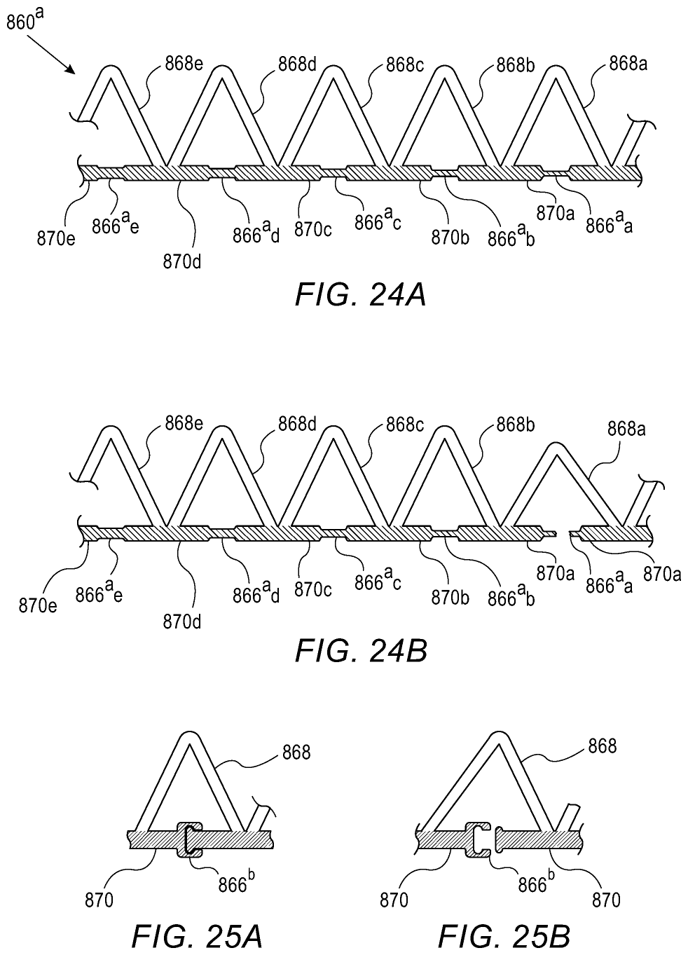

In some implementations, the separation zone 866 comprises a frangible portion. FIGS. 24A-24B show a portion of a frame belt 860a which includes a plurality of frangible portions 866^{a}. In FIG. 24A, all of the frangible portions 866^{a}ₐ, 866^{a}_{b}, 866^{a}_{c}, 866^{a}_{d} and 866^{a}ₑ are intact. FIG. 24B shows a successive state, which may be achieved in a valve partial expanded configuration as shown in FIG. 23B for example, in which the frangible zone 866^{a}a is disrupted (i.e., broken or torn), while other frangible portions, such as 866^{a}b, 866^{a}c, 866^{a}d and 866^{a}e, remain intact.

A frangible portion 866^{a} can be provided as a weakened portion along the frame belt 860^{a}. In applications that include expandable portions 868, the weakened zones can be provided along respective base portions 870. The frangible portion 866^{a} can be weakened by thinning thereof (relative to other, non-frangible portions of the frame belt 860^{a}), by inclusion of weakening features such as perforations, or by having material properties which are different than the material properties of other, non-frangible portions of the frame belt 860^{a}.

A frame belt 860^{a} can include a plurality of frangible portions 866^{a}, wherein at least two of the plurality of frangible portions 866^{a} are configured to disrupt (i.e., break or tear) in response to different tensioning force magnitudes applied thereto. FIG. 24A illustrates an example of a plurality of frangible portions 866^{a} provided with varying thickness, wherein frangible portion 866^{a}a is the thinnest frangible portion, and may therefore tear or break first, as shown in FIG. 24B, which may potentially correspond to a valve 120 applying a first critical tensioning force of interest, as shown in FIG. 23B. Frangible portion 866^{a}a is thicker than frangible portion 866^{a}a, frangible portion 866^{a}c is thicker than frangible portion 866^{a}b, frangible portion 866^{a}d is thicker than frangible portion 866^{a}c, and frangible portion 866^{a}e is thicker than frangible portion 866^{a}d. Thus, further expansion of the prosthetic valve 120 may apply increased tensioning forces on the frame belt 860^{a}, which may result in gradual tearing or breaking of subsequent frangible portions 866^{a}, as shown for example in FIG. 23C.

Each of a plurality of the frangible portions 866^{a} can be configured to disrupt in response to a different tensioning force magnitude, which may occur at a different expansion diameter of the prosthetic valve 120. In such configurations, the expansion force can be assessed by visually inspecting the amount of disrupted zones, represented by discontinuities of gapped zones along the radiopaque-marked frame belt 860^{a} under fluoroscopy. The amount of frangible portions 866^{a} may be determined according to a desired resolution of the monitored expansion forces.

The base portions 870 can be formed from an extensible material, which may extend around the valve 120 while the expandable portions 868 expand, until the point of their disruption. Alternatively, the base portions 870 can be provided in a folded or corrugating configuration that may unfold or straighten around the valve 120 while the expandable portions 868 expand, until the point of their disruption. It will be appreciated that the base portions 870 and the frangible portions 866 can be made of different materials than those of the expandable portions 868. For example, the expandable portions 868 may comprise super-elastic materials (Nitinol) or non-super-elastic materials (e.g., stainless steel or cobalt chromium alloys), while the base portions 870 can be provided with higher flexibility, for example provided in the form of a wire, cable, suture, string, or similar materials.

In some applications, the expandable portions 868 are radiopaque-marked such that a change in a geometrical characteristic thereof can be visible under fluoroscopy, indicative of a transition from a first state to a second state. For example, a height between an apex of an expandable portions 868 and the respective base portion 870 may serve as such a geometrical characteristic. As shown in FIG. 24A, the height of the expandable portion 868a may be equal to the height of all of the other expandable portions 868 in an intact state thereof. As further shown in FIG. 24B, the height of the expandable portion 868a, upon disruption of its respective frangible portion 860^{a}a, may be shorter that that of expandable portions 868b, 868c, 868d and 868e, whose corresponding frangible portion 860^{a} remain intact.

While the frame belt 860^{a} is shown in FIGS. 24A-24B with expandable portions 868, in alternative applications, a frame belt 860 can be provided without expandable portions 868. For example, a frame belt 860 can be provided as a flexible cable, wire, string, suture and the like, having frangible portions 866 disposed there-along, wherein the frame belt 860 can be coupled to the frame 120, directly or via intermediate components such as a skirt 136, 137 or a sleeve 132, 830 around the valve. The coupling can be facilitated via biocompatible adhesives, sutures and the like. Coupling can include a single coupling point, or a plurality of coupling points around the valve 120. For example, each frangible portion 866 can be disposed between two coupling points of the frame belt 860 to the valve 120, such as coupling to two adjacent junctions 130 positioned circumferentially on both sides of the respective frangible portion 866.

In some implementations, the separation zone 866 is provided in the form of a decouplable portion, configured to decouple when a tension force exceeding a predetermined magnitude is applied thereto. FIGS. 25A and 25B show a portion of a frame belt which includes a decouplable portion 866^{b} in a coupled and decoupled state thereof, respectively. In the illustrated example, a “key and lock” type of a decouplable portion 866^{b} can include a male part (e.g., a flanged or ball-type head) and a complementary female part (e.g., a slot or receptacle configured to tightly fit around the male part), which can be snap-fit or otherwise engaged with each other as shown in FIG. 25A. The male and female part can decouple from each other, as shown in FIG. 25B, in response to a tensioning force applied thereto (e.g., on both ends thereof), exceeding a specific threshold magnitude. Other types of a decouplable portion 866^{b} are similarly applicable, wherein a decouplable portion 866^{b} differs from a frangible portion 866^{a} only with respect to the type of disruption, which includes decoupling or releasing instead of breaking or tearing. Other than the type of disruption, decouplable portions 866^{b} may be utilized according to any of the embodiments described for frangible portions 866^{a}.

Since the frame belt is disposed around the frame 126, the struts 127 may mask such radiopaque-marked expansion force indicators 866, due to their own inherent radiopacity. In some applications, the radiodensity of the expansion force indicators 866 is higher than that of the struts 127 or other components of the prosthetic valve 120, so as to enable the radiopaque-marked expansion force indicators 866 to be visibly distinguishable, under fluoroscopy, from the frame 126 or other components of the valve 120.

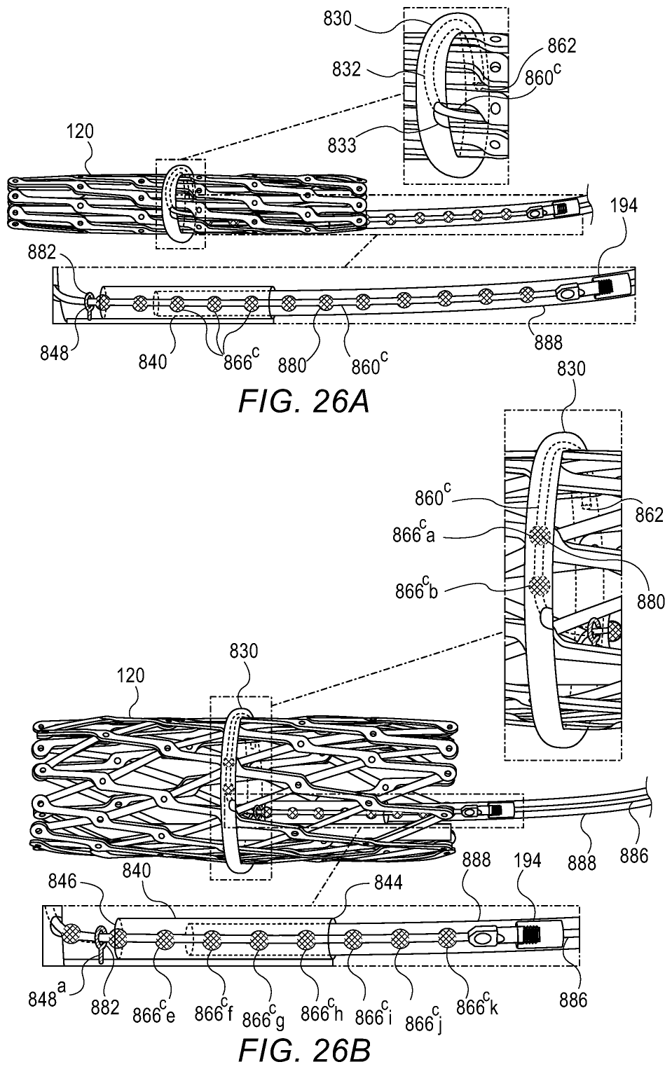

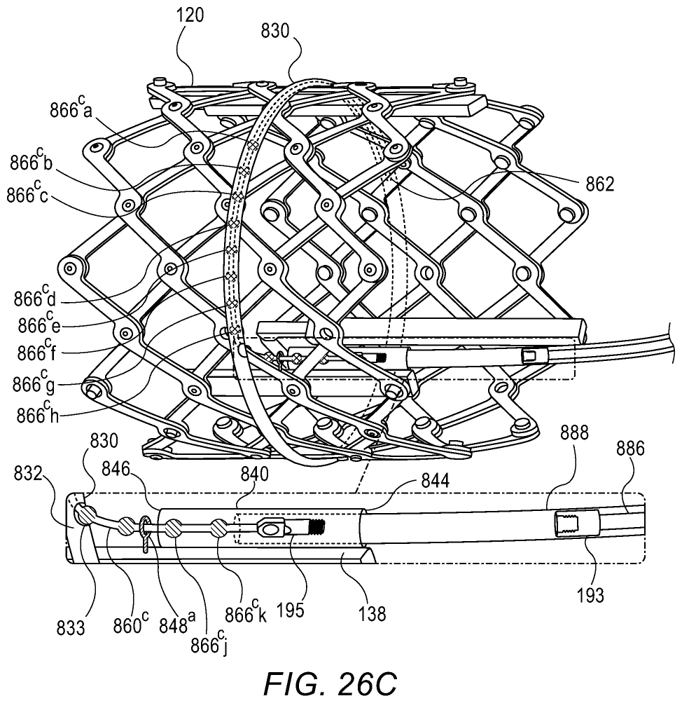

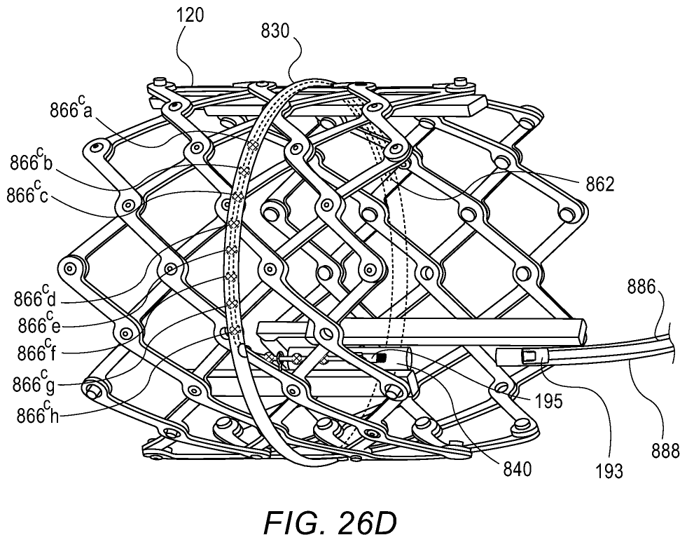

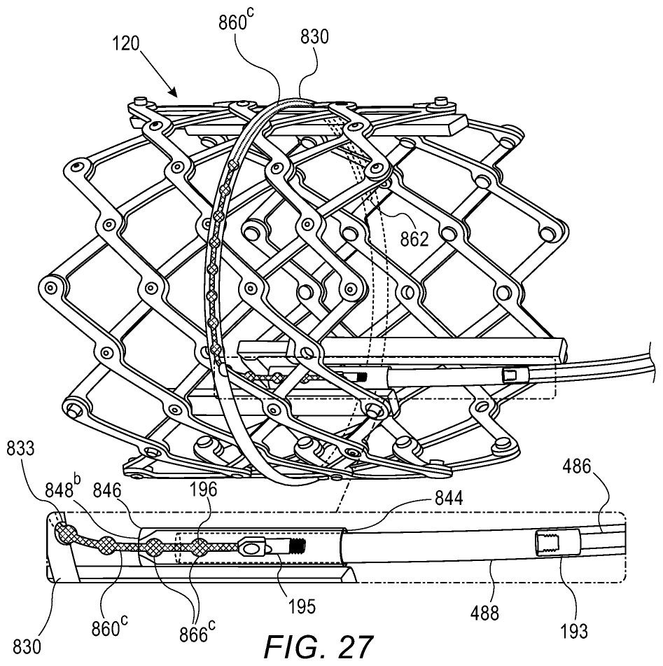

In an exemplary embodiment illustrated in FIG. 26A, a frame belt 860 with geometrical features 866 can be configured to extend at least partially around the prosthetic valve 120, such that the amount of the geometrical features 866 visible along the portion of the frame belt 860 circumscribing the valve 120 varies as a function of the valve's expansion diameter. In some implementations, the geometrical features 866 are provided in the form of beads 866^{a}. Each bead can include a separate indicator radiopaque marker 880, as shown in FIG. 26A. Alternatively, a continuous portion of the frame belt 860, including optionally the entire length thereof, can be marked with a radiopaque marker 196, wherein the geometrical features 866, such as the beads, are distinguishable from other radiopaque-marked regions due to their distinguishable size and/or shape, as shown for example in FIG. 27.

According to some embodiments, the delivery assembly 100 further comprises a restrictor 848, configured to allow passage of geometrical features 866 there-through upon application of a pulling force on the frame belt 860, exceeding a predetermined threshold. According to some embodiments, the prosthetic valve 120 comprises the restrictor 848. In some implementations, the restrictor 848 is provided in the form of an eyelet 848^{a}, that may restrict the passage of beads 866^{a} there-through. Specifically, the inner diameter of the eyelet 848^{a} can be slightly smaller than the outer diameter of the beads 866^{a}.

In the exemplary configuration shown in FIGS. 26A-26D, a frame belt 860, provided with a plurality of beads 866^{c}, includes a portion disposed partially around the circumference of the valve 120, and a portion which does not necessarily extend around the valve 120, shown in FIGS. 26A-26B to extend axially, in an orientation that can be substantially parallel to the longitudinal axis 121 of the valve 120. The frame belt 860 includes a frame belt first end 862, which can be attached directly or indirectly to the frame 126. In some applications, the portion of frame belt 860^{c} extending around the frame 126 is disposed in a sleeve, such as the circumferential sleeve 830 shown in FIGS. 26A-26D. In such cases, the circumferential sleeve 830 can be coupled to the frame 126, and the frame belt 860^{c} can enter into the circumferential sleeve lumen 832 through the circumferential sleeve opening 831. The frame belt first end 862 can be attached, for example by gluing, suturing, and the like, to the circumferential sleeve 830, defining the portion of frame belt 860^{c} extending around the frame 126 as the portion between the attachment point of the frame belt first end 862 to the circumferential sleeve 830 (shown in FIGS. 26A-26D on the rear side of the frame), and the circumferential sleeve opening 831.

According to some embodiments, the delivery assembly 120 further comprises a belt pull member 886 extending from the handle 110 and attached to, or integrally formed with, the frame belt 860. The belt pull member 886 can be provided in the form of a cable, a suture, a wire, and the like, and may be coupled to a pulling mechanism at the handle 110, maneuverable by an operator to retract the belt pull member 886, potentially along with at least a portion of the frame belt 860, when desired. According to some embodiments, the delivery assembly 120 further comprises a belt shaft 888 extending distally from the handle 110, allowing the belt pull member 886, potentially along with at least a portion of the frame belt 860, to extend through a lumen thereof.

In some cases, as shown in the configuration illustrated in FIGS. 26A-26B, a portion of the frame belt 860 can also extend through a guide member 840, that can be comprised in the valve 120 and may be utilized in combination with the belt shaft 888, similarly to any of the embodiments described for utilization thereof with a re-compression shaft 188.

In the exemplary configuration illustrated in FIG. 26A, a beaded portion of the frame belt 860^{c} can extend axially, through the lumens of the belt shaft 888 and/or the guide member 840, toward the circumferential sleeve opening 831, and an un-beaded portion of frame belt 860^{c} can extend circumferentially around the frame 126, within the circumferential sleeve lumen 832, in a crimped configuration of the prosthetic valve 120. In some applications, as shown in FIG. 26A, the eyelet 848^{a} can be positioned between the guide member distal end 846 and the circumferential sleeve opening 831, while the frame belt 860^{c} extends there-through. For example, the eyelet 848^{a} can be attached to the frame 126 or to the actuator assembly 138 (e.g., to the actuator outer member 140), distal to the guide member distal end 846, and potentially aligned therewith.

FIG. 26B shows a partially expanded state of the prosthetic valve 120. In this state, some of the beads, such as beads 866^{c}a and 866^{c}b, are positioned around the circumference of the valve 120, within the circumferential sleeve 830, while other beads, such as beads 866^{c}e, 866^{c}f, 866^{c}g, 866^{c}h, 866^{c}i, 866^{c}j and 866^{c}k, remain out of the circumferential sleeve 830, for example disposed within the guide member 840 and/or the belt shaft 888.

The circumferential sleeve 830 can comprise a radiolucent material or include a cut-out window, so as to allow visibility of radiopaque marked beads 866^{c} disposed therein. Thus, the number of beads 866^{c} circumferentially disposed around the valve 120, which can be visible under fluoroscopy, can be indicative of the valve expansion diameter. For example, FIG. 26C shows the valve 120 further expanded, potentially to a final expansion diameter, wherein a larger number of beads, such as beads 866^{c}a, 866^{c}b, 866^{c}c, 866^{c}d, 866^{c}e, 866^{c}f, 866^{c}g and 866^{c}h, are positioned around the circumference of the valve 120, while a lower number of beads, such as beads 866^{c}j and 866^{c}k, remain out of the circumferential sleeve 830.

The sleeve 830 can advantageously serve as a guide member, directing the geometrical features 866 (such as beads 866^{c}) around the circumference of the valve 120 upon expansion thereof. While a circumferential sleeve 830 is described and illustrated for use in combination with a frame belt 860 provided with geometrical features 866, it will be clear that a sleeve 132, that can be attached to, or integrally formed with, a skirt (e.g., an outer skirt 137), can be used instead in the same manner. Moreover, in some applications, the sleeve can be replaced with other means of guiding the frame belt 860 circumferentially around the valve 120, such as suture loops (not shown) looped around the frame 126 (e.g., around struts 127 of junction 130), through which the frame belt 860 may slide. In yet alternative applications, a sleeve or other type of guiding means is used, and the frame belt 860 can be attached, at least at the frame belt first end 862, to the frame 126, directly (e.g., to a strut 127 or a junction 130) or indirectly (e.g., to a skirt).

According to some embodiments, the belt pull member 886 may be attached to the frame belt 860 via a connector 194, which can be, in some variants of the embodiments, a releasable connector 194. For example, once the expansion procedure is complete, as shown in FIG. 26C, the proximal connector element 193 can be released from the distal connector elements 195, and the belt pull member 886 can be retracted with the proximal connector element 193 attached thereto, while the distal connector elements 195, potentially with a portion of the frame belt 860 attached thereto, can remain with the expanded valve 120, for example disposed within the guide member 840. As shown in FIG. 26D, a subsequent step can include retraction of the belt shaft 888 from the valve 120 and the guide member 840. This process can be implemented according to any of the embodiments of a re-compression assembly 180 having a releasable connector 194, described and illustrated in conjunction with FIGS. 7A-7C.

While FIG. 26A shows an un-beaded portion initially disposed circumferentially around the valve 120 in the crimped configuration, it will be clear that in alternative configuration, beads 866^{c} can be disposed around the valve 126 even in a crimped configuration. In such cases, the initial amount of beads 866^{c} disposed around the valve 120 in this configuration, may be indicative of a crimped diameter, and expansion force can be estimated by a corresponding increased number of beads 866^{c}.

In some applications, the guide member 840, and/or at least a portion (e.g., a distal portion) of the belt shaft 888, include a radiopaque zone (for example, around their outer surfaces), configured to hide or mask the geometrical features 866 disposed within their lumens, from visibility under fluoroscopy. Such applications can advantageously simplify visual identification of the number of geometrical features 866 (e.g., beads 866^{c}) around the valve 120, as only these beads are visible and unmasked under fluoroscopy.

While a plurality of beads 866^{c} are illustrated, alternative configurations can include a single geometrical feature, such as a single bead 866^{c} which may be hidden from view, or otherwise positioned in an initial position, which may change as the valve expands. Such configurations may be applicable for diameter detection instead of force measurement, for example a maximal expansion diameter, such that exposure of the bead 866^{c}, or alternatively, positioning thereof in a second distinguishable position, may be indicative of over-expansion.

As mentioned above, measurement mechanisms situated at the handle 110, that rely on force transmission from a region adjacent the prosthetic valve 120 to the handle 110, may result in inaccuracies in valve diameter estimation due to lengthening and/or multiple bend-regions or twists that might be formed along the tortuous vasculature of the patient, and may require additional means for compensating for such inaccuracies, such as springs or pulley assemblies, as described throughout the current disclosure. A frame belt equipped with expansion force indicators, including indicators that comprise separation zones and/or geometrical features (e.g., beads) according to any of the embodiments disclosed herein, might be advantageous as they provide discrete transitions between states of the expansion force indicators (e.g., breaking/tearing, “jumping” through a restrictor, etc.), achieved only once respective circumferential force threshold are applied thereto by the expanding frame 126.

According to some embodiments, frictional forces between any of the belt pull member 886 and/or the frame belt 860, with any of the belt shaft 888, the guide member 840 and/or the sleeve 132, 830, are set to be lower than the estimated circumferential forces exerted by the frame 126 on the frame belt 860. This may be achieved by an appropriate selection of material properties, manufacturing such components with desired surface roughness, or coating with low-friction layers.

While not shown explicitly, a frame belt 860 with geometrical features 860 can be utilized without passing through a guide member 840, and/or without passing through a belt shaft 888. Similarly, a frame belt 860 with geometrical features 860 can extend through a guide member 840, and/or through a belt shaft 888, which do not mask the radiopaque markers around the geometrical features 866. In such configurations, geometrical features 866, such as beads 866, may be visible both when positioned around the prosthetic valve 120 (for example, within a sleeve), or along a portion of the frame belt 860 which does not circumscribe the valve 120 (e.g., proximal to the sleeve). A viewer (e.g., clinician) can distinguish, in such cases, between a circumferential orientation of some beads 866^{c} and a non-circumferential orientation of other beads, when viewed under fluoroscopy. For example, with reference back to FIG. 26B, it may be possible to identify that the beads 866^{c}a and 866^{c}b define a first orientation, which is the circumferential orientation, and that the beads 866^{c}a, 866^{c}b, 866^{c}c, 866^{c}d, 866^{c}e, 866^{c}f, 866^{c}g and 866^{c}h, define a second orientation, which is a non-circumferential orientation, shown as an axial orientation which is substantially perpendicular to the first orientation. Once the circumferential orientation is identified, the number of beads visible in that orientation can be indicative of the valve expansion diameter.

According to some embodiments, the restrictor 848 comprises a reference radiopaque marker 882. For example, the eyelet 848^{a} can include a reference radiopaque marker 882. Since the eyelet 848^{a} is rigidly attached to one frame component (e.g., the actuator outer member 140), the reference radiopaque marker 882 remains immovable with respect to the outflow end 123. A viewer can distinguish, in such cases, between beads 866^{c} positioned on both sides of the reference radiopaque marker 882, when viewed under fluoroscopy. For example, with reference back to FIG. 26B, it may be possible to identify that the beads 866^{c}a and 866^{c}b are positioned distal to the radiopaque-marked eyelet 848^{a}, and that the beads 866^{c}a, 866^{c}b, 866^{c}c, 866^{c}d, 866^{c}e, 866^{c}f, 866^{c}g and 866^{c}h, are positioned proximal to the radiopaque-marked eyelet 848^{a}. The number of beads 866^{c} visible on a specific side of the reference radiopaque marker 882, such as the number of beads 866^{c} distal to the reference radiopaque marker 882, can be indicative of the valve expansion diameter.

In some implementations, the restrictor 848 is implemented as a narrow opening through which the frame belt 860 extends. For example, FIG. 27 shows a configuration which is similar to that shown in FIG. 26C, except that the guide member distal end 846 includes a guide member constriction 848^{b}, which may be formed as an inwardly flanged or tapering opening, having an opening diameter which is slightly smaller than the outer diameter of the beads 866^{a}, therefore acting as an alternative to the eyelet 848^{a}. While shown at the guide member distal end 846, in alternative variations, the guide member constriction 848^{b} can be formed as a narrowing at the guide member proximal end 844, or as a local narrowing anywhere else along the guide member lumen 842. Similarly, while not shown explicitly, a narrowing with an inner diameter slightly smaller than that of the geometrical features 866 can be formed at the circumferential sleeve opening 831, formed as a local narrowing within the circumferential sleeve lumen 832, or as a narrowing at the opening of, or within the lumen of, the belt shaft 888.

According to some embodiments, a frame belt 860 provided with geometrical features 866, circumscribes the prosthetic valve 120 such that the entire length of its beaded portion is disposed around the frame 126. For example, FIG. 28A shows a prosthetic valve 120 in a crimped configuration, provided with a circumferential sleeve 830 circumscribing a portion of the circumference of the valve 120. A frame belt 866^{c} is also disposed around the prosthetic valve 120, such that a first portion thereof, which is an un-beaded portion shown in FIG. 28A, extends out of the circumferential sleeve 830, and another beaded portion is disposed within the circumferential sleeve lumen 832. The restrictor 848 can be provided in the form of a sleeve constriction 848^{c}, at the opening of the sleeve 830 through which the frame belt 866^{c} extends.

When the prosthetic valve expands, as shown in FIG. 28B, at least some of the beads, such as beads 866^{c}a, 866^{c}b, 866^{c}c and 866^{c}d, are pulled out of the circumferential sleeve 830, while other beads, such as beads 866^{c}e, 866^{c}f and 866^{c}g, remain within the sleeve 830.

The circumferential sleeve 830 includes a circumferential sleeve first end 834, which can be an enclosed end portion of the sleeve, and may be affixed to the frame 126 (e.g., to a junction 130), via an affixing member 850. The affixing member 850 can include a suture, a biocompatible adhesive, and the like. The circumferential sleeve 830 further includes a circumferential sleeve second end 834, which can be an opening through which the frame belt 860 may extend. The circumferential sleeve 830 can be coupled to the frame 126 along additional coupling points thereof via slidable attachment members 852 (see FIG. 28B), which can be provided in the form of suture loops, bands, and the like, allowing the sleeve 830 to slide there-through while the valve 120 expands.

The frame bolt first end 862 can be similarly affixed to the frame 126 (e.g., to a junction 130) via an affixing member 850. The opposite, frame bolt second end 864, can be a free end disposed within the circumferential sleeve lumen 832, enabling the frame bolt 860 to slide through, and partially out of, the sleeve 830, as the valve 120 expands.

While FIG. 28A shows an un-beaded portion exposed out of the sleeve 830 in the crimped state, it will be clear that in alternative configuration, beads 866^{c} can be disposed around the valve 126 and out of the sleeve 830 in this state. In such configurations, the initial amount of beads 866^{c} disposed out of the sleeve 130 in this state, may be indicative of a crimped state of the valve, and an increased number of beads 866^{c} that are further distinguishable due to repositioning thereof out of the sleeve 830, are indicative of the force applied by the valve during expansion thereof, exceeding threshold values required to displace the beads 866^{c}.

In some applications, the circumferential sleeve 830 comprises a radiopaque zone, for example around its outer surface, configured to hide or mask the beads 866^{c} disposed within its lumen from visibility under fluoroscopy. Thus, in the state shown in FIG. 28A, all of the beads 866^{c} shown to be positioned within the circumferential sleeve lumen 832 may be hidden from view, and only beads pulled out of the sleeve 830, such as beads 866^{c}a, 866^{c}b, 866^{c}c and 866^{c}d in FIG. 28B, may be exposed and visible under fluoroscopy, such that the amount of visible beads can be indicative of the valve expansion diameter.

In alternative applications, the beads 866^{c} can be visible through the sleeve 830 as well. In such applications, the valve 120 can further comprise a reference radiopaque marker (not shown in FIGS. 28A-28B). For example, the circumferential sleeve second end 836 can include a reference radiopaque marker. A viewer can distinguish, in such cases, between beads 866^{c} positioned on both sides of the reference radiopaque marker, when viewed under fluoroscopy. In a further variation of the applications, the valve 120 can be devoid of a sleeve 130 (application not shown), but provided with a restrictor 848 such as an eyelet 848^{a}, rigidly attached to the frame 126 and having a reference radiopaque marker thereon, wherein the frame belt 860 extends there-through so as to enable a viewer to distinguish between beads 866^{c} positioned on both sides of the reference radiopaque marker in a similar manner.

While not explicitly shown, it will be understood that all of the embodiments described and illustrated for the beads 866^{c} in conjunction with FIGS. 26A-28B, are similarly applicable in general to other geometrical features 866, including, but not limited to: knots, balls, ribs and spokes.

FIGS. 29A and 29B show configurations which are similar to the configurations shown in FIGS. 28A and 28B, respectively, except that the geometrical features 866 are provided in the form of belt ratcheting teeth 866^{d}, and the restrictor 848 is provided in the form of complementary sleeve ratcheting teeth 848^{d} comprised within the circumferential sleeve lumen 832. FIG. 29B shows a portion of the belt ratcheting teeth 866^{d}a exposed out of the sleeve 830 in an expanded state of the valve 120, and a portion of the belt ratcheting teeth 866^{d}b that may remain within the sleeve 830. The amount of radiopaque-marked ratcheting teeth 866^{d}a exposed out of the sleeve 130 may be indicative of the valve expansion diameter. Moreover, any embodiment that relates to the expansion belt 860^{c} provided with beads 866^{c}, as described and illustrated in conjunction with FIGS. 28A-28B, applies with equal force to the expansion belt 860^{d} provided with belt ratcheting teeth 866^{d}, as illustrated in FIGS. 29A-29B.

While a plurality of sleeve ratcheting teeth 848^{d} are shown in FIGS. 29A-29B, it will be clear that a restrictor 848 may be similarly implemented as a single tooth or pawl, configured to apply a ratcheting mechanism to the belt ratcheting teeth 866^{d}.

Since the frame belt 860 is required to provide an indication of the valve's expansion force during the implantation procedure, and is no longer needed once the valve is positioned at the implant site, it can comprise, according to some embodiments, a bio-resorbable material, such as a bio-resorbable polymer, configured to dissolve over time. The resorption rate of the bio-resorbable frame belt 860 can be controlled by a variety of parameters, such as the polymer material, additives, processing and the like. Some examples of polymeric resorbable materials include, but are not limited to: Polylactide (PLA), Poly-L-lactide (PLLA), Polyglycolide (PGA), Poly-e-Caprolactone (PCL), Trimethylene carbonate (TMC), Poly-DL-lactide (PDLLA), Poly-b-hydroxybutyrate (PBA), Poly-p-dioxanone (PDO) Poly-b-hydroxypropionate (PHPA) and Poly-b-malic acid (PMLA). Preferably, the bio-resorbable material is comprised in the frame belt 860 in a manner that will not interfere with its radiopacity, according to some embodiments elaborated herein above.

According to some embodiments, the frame belt 860 comprises at least one electrically conductive expansion force indicator. For example, an expansion force indicator 866 can comprise a stretch sensor, configured to change is electrical resistivity when stretched over the frame 126 during expansion thereof. The stretch sensor can be operatively coupled, via a transmission line, to a control unit and display in the handle 110. The transmission line can be implemented in a similar manner to that of the belt pull member 886, which may be releasably coupled via a releasable electrical connector similar to the releasable connector 194. The transmission line and the releasable connector may include various electrically conductive materials, such as copper, aluminum, silver, gold, and various alloys such as tentalum/platinum, MP35N and the like. An insulator can surround the transmission line and/or releasable connector. The insulator can include various electrically insulating materials, such as electrically insulating polymers.

In use, expansion of the frame 126 may result in elongation or stretching of the stretch sensor, which may generate corresponding electrical signals in the form of current, voltage, resistance, or change in the same. The signals may be electrically conducted to a control circuitry in the handle 110, potentially via terminals connected to electrically conductive releasable connector and transmission line, and may be interpreted and display on a display 116.

The transmission line may be releasably attached to the frame belt in a manner similar to the configuration exemplified in FIGS. 26A-26D, wherein the frame belt may include a stretch sensor (not shown) instead of beads 866^{c}, wherein the releasable electrical connector is represented by the releasable connector 194, and wherein the transmission line is represented by the belt pull member 886, which may extend through a transmission line shaft represented by the belt shaft 888. According to some embodiments, when the electrically conductive proximal connector element 193 is coupled to the electrically conductive distal connector element 195 (as shown in FIGS. 26A-26B), the transmission line shaft 888 is hermetically coupled to the guide member 840, in a manner that seals the electrically conductive connector 194 from surrounding blood flow. Thus, when the transmission line is decoupled from the frame belt, in a configuration similar to that shown in FIG. 26C, the exposed end of the proximal connector element 193 remains isolated from the surrounding environment of the blood flow, which allows the transmission line to be detached safely pulled through the shaft 888 while avoiding the risk of exposing the surrounding blood flow or other tissues to electrical current thereof.

The shaft 888 may be threadedly engaged with the guide member 840. Once the transmission line is detached from the frame belt and pulled away therefrom, the shaft 888 may be rotated so as to detach from the guide member 840. According to some embodiments, the transmission line is pulled along a sufficient distance prior to disengaging the shaft 888 from the guide member 840, such that once the shaft 88 is detached and retracted from the valve 120, in a manner similar to that shown in FIG. 26D, the transmission line cannot be exposed to the blood flow flowing through the lumen of the shaft 888.

Utilization of a frame belt 860 provided with at least one expansion force indicator in the form of a stretch sensor, or other types of sensing elements configured to change an electrical property thereof (e.g., resistivity or capacitance) in response to stretch forces imparted thereon by an expanding valve 120, may be advantageous over visual detection of radiopaque-marked expansion force indicators 866, according to any of the embodiments described and illustrated in conjunction with FIGS. 23A-29B, as it avoids potential interference that may result from the inherent radiopacity of the frame 126.

While the embodiments are described and illustrated throughout the current disclosure for use with a mechanically expandable valve 120, it will be clear that methods for providing real-time estimation of the valve diameter, based on a relationship between the expansion diameter and a dimensionless parameter, as well as methods and devices for providing real-time estimation of the valve expansion force based on frame belts 860 according to any of the embodiments disclosed herein, may be similarly used in combination with other valve types, such as balloon expandable valves or self-expandable valves. However, conventional balloon-expandable valves and self-expandable valves are typically inflated or expanded during a short time period (e.g., in a burst), in a manner which provides limited control of valve expansion. In contrast, utilization of the above mentioned imaging methods, or utilization of frame belts 860, in combination with mechanically expendable valves 120, is advantageous since the mechanical expansion mechanism (for example—as described in conjunction with FIGS. 4A-C) provides a higher degree of control over the rate and extent of valve expansion, enabling the clinician to adjust expansion diameter, responsive to real-time feedback regarding valve diameter and/or expansion force.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. No feature described in the context of an embodiment is to be considered an essential feature of that embodiment, unless explicitly specified as such.

Although the invention is described in conjunction with specific embodiments thereof, it is evident that numerous alternatives, modifications and variations that are apparent to those skilled in the art may exist. It is to be understood that the invention is not necessarily limited in its application to the details of construction and the arrangement of the components and/or methods set forth herein. Other embodiments may be practiced, and an embodiment may be carried out in various ways. Accordingly, the invention embraces all such alternatives, modifications and variations that fall within the scope of the appended claims.

1. A delivery assembly, comprising:
a prosthetic valve movable between a radially compressed configuration and a radially expanded configuration; and
a delivery apparatus comprising:
a handle;
a delivery shaft extending distally from the handle; and
a re-compression assembly comprising:
a re-compression shaft extending through a lumen of the delivery shaft; and
a re-compression member extending through a lumen of the re-compression shaft and having a loop portion configured to circumscribe the prosthetic valve, the loop portion comprising at least one radiopaque marker;
wherein relative movement between the re-compression member and the re-compression shaft is configured to tighten the loop portion around the prosthetic valve, thereby radially compressing the prosthetic valve.

2. The delivery assembly of claim 1, wherein the at least one radiopaque marker comprises a plurality of radiopaque markers, spaced from each other along at least a portion of the loop portion.

3. The delivery assembly of claim 1, wherein the at least one radiopaque marker comprises radiopaque bands.

4. The delivery assembly of claim 1, wherein the at least one radiopaque marker comprises a plurality of radiopaque markets spanning along a portion of the loop portion that is at least as long as half of the perimeter of the prosthetic valve when the prosthetic valve is in the radially expanded configuration.

5. The delivery assembly of claim 1, wherein the at least one radiopaque marker is disposed on the loop portion at a position corresponding to a contact region between the loop portion and the perimeter of the prosthetic valve.

6. The delivery assembly of claim 5, wherein the at least one radiopaque market is disposed along a minimal marking length of the loop portion, wherein the minimal marking length is at least as great as the perimeter of the prosthetic valve in the radially expanded configuration.

7. The delivery assembly of claim 1, wherein the at least one radiopaque marker comprises radiopaque coating.

8. The delivery assembly of claim 1, wherein the re-compression member further comprises a releasable connector, wherein the releasable connector comprises a proximal connector element and a distal connector element releasably attached to each other, wherein the re-compression member comprises a proximal segment coupled to the proximal connector element, and wherein the loop portion is coupled to the distal connector element.

9. The delivery assembly of claim 1, wherein the prosthetic valve comprises a guide member, and wherein at least a portion of the re-compression member extends through a lumen of the guide member.

10. The delivery assembly of claim 1, wherein the prosthetic valve further comprises a sleeve disposed around at least a portion of the circumference of the prosthetic valve, and wherein at least a portion of the loop portion extends through the sleeve.

11. A delivery assembly, comprising:
a prosthetic valve movable between a radially compressed configuration and a radially expanded configuration; and
a delivery apparatus comprising:
a handle;
a delivery shaft extending distally from the handle; and
a re-compression assembly; comprising:
a re-compression shaft extending through a lumen of the delivery shaft, and comprising at least one reference radiopaque marker,
a re-compression member comprising at least one indicator radiopaque marker, a proximal segment, and a loop portion;
wherein the re-compression member extends through a lumen of the re-compression shaft;
wherein the loop portion extends distally from the re-compression shaft;
wherein relative movement between the re-compression member and the re-compression shaft is effective is configured to tighten the loop portion around the prosthetic valve, thereby radially compressing the prosthetic valve; and
wherein position of the at least one indicator radiopaque marker, relative to that of the at least one reference radiopaque marker, is indicative of the diameter of the prosthetic valve.

12. The delivery assembly of claim 11, wherein the at least one reference radiopaque marker comprises a plurality of reference radiopaque markers, wherein each reference radiopaque marker of the plurality of reference radiopaque markets is associated with a different diameter of the prosthetic valve, and wherein alignment of a given indicator radiopaque marker with any one of the plurality of reference radiopaque markers is indicative of the diameter associated with a reference radiopaque aligned with the given indicator radiopaque marker.

13. The delivery assembly of claim 11, wherein the proximal segment comprises the at least one indicator radiopaque marker.

14. The delivery assembly of claim 11, wherein the re-compression member further comprises a connector coupled to the proximal segment and the loop portion.

15. The delivery assembly of claim 14, wherein the connector comprises the at least one indicator radiopaque marker.

16. The delivery assembly of claim 14, wherein the connector is a releasable connector, wherein the connector comprises a proximal connector element and a distal connector element releasably attached to each other, wherein the proximal segment is coupled to the proximal connector element, and wherein the loop portion is coupled to the distal connector element.

17. The delivery assembly of claim 11, wherein the prosthetic valve comprises a guide member, and wherein at least a portion of the re-compression member extends through a lumen of the guide member.

18. The delivery assembly of claim 11, wherein the prosthetic valve further comprises a sleeve disposed around at least a portion of the circumference of the prosthetic valve, wherein at least a portion of the loop portion extends through the sleeve.

19. The delivery assembly of claim 11, further comprising a plurality of actuation arm assemblies coupled to the prosthetic valve, wherein the plurality of actuation arm assemblies is configured to move the prosthetic valve between the radially compressed and the radially expanded configurations, wherein the plurality of actuation arm assemblies comprises a plurality of loop attachment members, and wherein the loop portion is coupled to, and extends between, the plurality of loop attachment members.

20. The delivery assembly of claim 11, wherein the handle further comprises a spring connected to the proximal segment, wherein the handle is configured to apply an axially oriented pull-force on the proximal segment, and wherein the pull-force is sufficient to apply a minimal tension magnitude to the loop portion.

21. The delivery assembly of claim 11, wherein the handle further comprises a pulley assembly, the pulley assembly comprising:
a first pulley attached to the handle via a first pin and rotatable around the first pin; and
a second pulley attached to the handle via a second pin and rotatable around the second pin;
wherein the proximal segment is routed partially around the first pulley and around the second pulley; and
wherein the pulley assembly is configured to apply a minimal tension magnitude to the loop portion.
